(19) United States
(12) Patent Application Publication
(10) Pub. No.: US 2009/0076047 A1
(43) Pub. Date: Mar. 19, 2009

(54) 2-Substituted Hydroxylaminopyrimidine, Method for the Production and the Use Thereof in the Form of Pesticides

(76) Inventors: Joachim Rheinheimer, Ludwigshafen (DE); Thomas Grote, Wachenheim (DE); Bernd Muller, Frankenthal (DE); Jan Klaas Lohmann, Mannheim (DE); Wassilios Grammenos, Ludwigshafen (DE); Udo Hunger, Mannheim (DE); Frank Schieweck, Bad Durkheim (DE); Sarah Ulmschneider, Bad Durkheim (DE); Jochen Dietz, Mannheim (DE); Jens Renner, Bad Durkheim (DE); John-Bryan Speakman, Bobenheim (DE); Maria Scherer, Landau (DE); Siegfried Strathmann, Limburgerhof (DE); Reinhard Stierl, Freinsheim (DE)

Correspondence Address: BRINKS, HOFER, GILSON & LIONE, P.O. BOX 1340, MORRISVILLE, NC, 27560, US

(21) Appl. No.: 12/067,833

(22) PCT Filed: Sep. 21, 2006
(86) PCT No.: PCT/EP2006/066572

§ 371 Date: Mar. 24, 2008

(30) Foreign Application Priority Data
Sep. 28, 2005 (DE) 102005046592.7

(51) Int. Cl. A01N 43/54; C07D 403/04; C07D 239/48; A01N 43/80; A01P 3/00; A01N 43/653; A01N 43/76; C07D 239/42

(52) U.S. Cl. 514/275; 544/324; 544/325; 544/326

(57) ABSTRACT

2-Substituted pyrimidines of the formula I,

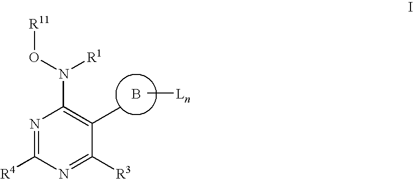

where the indices and substituents are as defined in the description.

The present invention relates to 2-substituted pyrimidines of the formula I

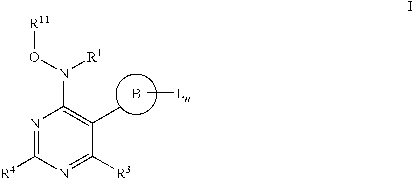

where the indices and substituents are as defined below:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
   R¹ and R¹¹ together with the atoms to which they are attached may also form a five-, six- or seven-membered saturated or unsaturated heterocycle which may contain a further heteroatom from the group consisting of O, N and S as ring member, where
   R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; where R² is:
   R² halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, where the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; where m, A, A′ and A″ are:
      m 0, 1 or 2;
      A, A′, A″ independently of one another hydrogen, C₁-C₆-alkyl, C₂-C₆-alkenyl, C₂-C₆-alkynyl, C₃-C₈-cycloalkyl, C₃-C₈-cycloalkenyl, phenyl, where the organic radicals may be partially or fully halogenated and/or may be mono- or polysubstituted by nitro, cyanato, cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, where the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, where R⁴ may be partially or fully halogenated and/or may carry one, two, three or four identical or different groups R^{u}:
   R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; where m, A, A′, A″ are as defined above;
      R⁴ may furthermore be:
      cyano, C₁-C₈-alkoxy, (=Z)OR^{a}, C(=Z)NR^{z}R^{b}, C(=Z)NR^{a}—NR^{z}R^{b},
      C(=Z)R^{a},
      CR^{a}R^{b}—OR^{z}, CR^{a}R^{b}—NR^{z}R^{c},
      ON(═CR^{a}R^{b}), O—C(=Z)R^{a},
      NR^{a}R^{b′}, NR^{a}(C(=Z)R^{b}), NR^{a}(C(=Z)OR^{b}), NR^{a}(C(=Z)-NR^{z}R^{b}),
      NR^{a}(N═CRCR^{b}), NR^{a}—NR^{z}R^{b}, NR^{z}—OR^{a}; where
      Z is O, S, NR^{d}, NOR^{d} or N—NR^{z}R^{c};
   R^{b′} is C₁-C₆-alkyl, C₂-C₆-alkenyl, C₂-C₆-alkynyl, C₃-C₆-cycloalkyl or C₄-C₆-cycloalkenyl;
   R^{a}, R^{b}, R^{c}, R^{d} independently of one another are hydrogen or have one of the meanings mentioned for R^{b′};
   R^{z} has the same meanings as R^{a} and may additionally be —CO—R^{d} or —COO—R^{d};
   where die aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
   R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
   and where two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S;

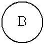

is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; where m, A, A′, A″ are as defined above;
   where the aliphatic and alicyclic groups of the radical definitions of L may carry one, two, three or four groups R^{L}:
   R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; where m, A, A′, A″ are as defined above;
and
n is 1, 2, 3, 4 or 5;
   and agriculturally acceptable salts of the compounds I.

Furthermore, the present invention relates to compositions comprising at least one of the compounds according to the invention, to processes for preparing these compounds, to intermediates for preparing these compounds and to the agriculturally acceptable salts thereof, to the preparation of the intermediates and to the use of the compounds according to the invention for controlling phytopathogenic fungi and for controlling animal pests.

Depending on the substitution pattern, the compounds according to the invention may have one or more centers of chirality, in which case they are present as enantiomer or diastereomer mixtures. The invention provides both the pure enantiomers or diastereomers or rotamers and mixtures thereof. Suitable compounds of the formula (I) also include all possible stereoisomers (cis/trans isomers) and mixtures thereof. The compounds according to the invention can be present in different crystal modifications, which may differ in their biological activity. They also form part of the subject matter of the present invention.

2-substituted pyrimidines having fungicidal action are known from the literature (WO 01/096314, WO 02/074753, WO 03/043993, WO 04/103978).

However, in many cases the action of the abovementioned pyrimidines is not entirely satisfactory. Accordingly, it was an object of the present invention to provide further compounds having fungicidal activity.

Surprisingly, this object is achieved by the 2-substituted pyrimidines according to the invention. Also provided in the context of the present invention are processes for their preparation and compositions comprising them, their use for controlling phytopathogenic fungi and their use for controlling animal pests.

According to the present invention, agriculturally acceptable salts include in particular the salts of those cations and the acid addition salts of those acids whose cations and anions, respectively, have no adverse effect on the pesticidal action of the compounds according to the invention.

Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may bear from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are for example chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting the compounds according to the invention with an acid of the corresponding anion, preferably hydrochloric acid, hydrobromic acid, sulfuric acid, phosphoric acid or nitric acid.

The compounds according to the invention can be obtained by various routes.

The compounds according to the invention can be prepared, for example, from correspondingly substituted phenylmalonates 2. These are known or obtainable analogously to the known substances.

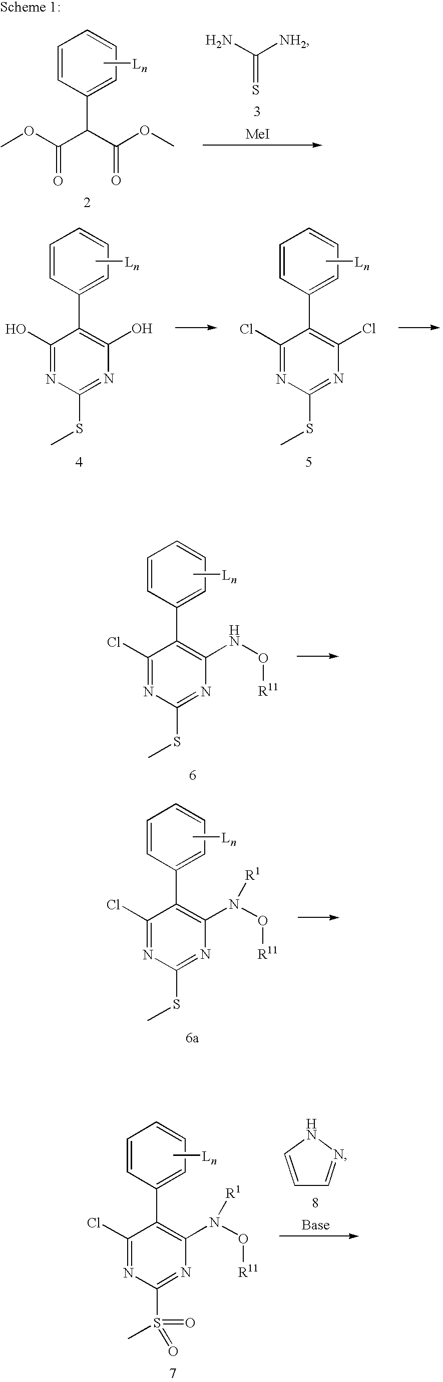

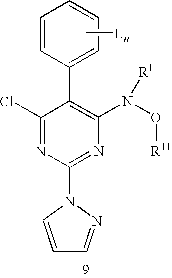

Using thiourea (3) and a methylating agent or using S-methylisothiourea, phenylmalonates 2 can be converted into the dihydroxypyrimidine derivatives 4 (see Scheme 1). Suitable methylating agents are, for example, methyl iodide, methyl bromide or dimethyl sulfate.

Here, preference is given to using a solvent which is inert under the reaction conditions and in which the reactants are sufficiently soluble. The reaction temperature is preferably between −20° C. and 150° C., with preference between 0° C. and 100° C.

The dihydroxypyrimidines 4 obtainable in this manner can then be halogenated by customary methods, for example chlorinated to give the dichloropyrimidines 5, as illustrated in Scheme 1. For the chlorination, it has been found to be particularly suitable to use phosphorus oxychloride, if appropriate with addition of an amine such as diethylaniline, an amine hydrochloride such as trimethylammonium chloride, or dimethylformamide. It may be advantageous to carry out the reaction at elevated temperature, for example under reflux of the phosphorus oxychloride, to increase the conversion. Bromination may be carried out by known methods using, for example, POBr₃.

The dihalopyrimidines formed, such as, for example, dichloropyrimidines 5 (Scheme 1) can then be substituted further by various routes. Here, it has been found that the regioselectivity often depends unexpectedly strongly on the chosen reaction partners and conditions. In the route shown in Scheme 1, the hydroxylamine is attached nucleophilically in the 4-position. Here, it is possible to use both monoalkylated (H—NH—O—R¹¹, as shown in Scheme 1) and dialkylated hydroxylamines (R¹—NH—O—R¹¹). Since the latter are frequently not commercially available, it has been found to be expedient to alkylate the compounds 6, if appropriate, to 6a (as shown in Scheme 1). Frequently, it is possible to avoid this by the separate synthesis of the corresponding disubstituted hydroxylamine. A further advantage of the subsequent introduction of the alkyl group is that the pyrimidine radical replaces the protective group which is otherwise in most cases required at the hydroxylamine, so that the synthesis is shortened by this procedure.

The thiolate group (C₁-C₆-alkylthio, in Scheme 1 shown in an exemplary manner as a methyl group) in the 2-position of the compound 6a is oxidized to the C₁-C₆-alkylsulfonyl (C₁-C₆-alkylS[═O]₂—) group of the compound 7 and thus converted into a leaving group for further exchange reactions. Hydrogen peroxide or peracids of organic carboxylic acids have been found to be particularly suitable oxidizing agents. However, the oxidation can also be carried out using, for example, selenium dioxide.

For introducing a heterocyclic radical R⁴ into the 2-position of the compound 7, it may be possible, depending on the nucleophilicity, to use the heterocycle directly (such as, for example, pyrazole, triazole). In these cases, an auxiliary base is generally employed. Heterocyclic substituents can also be introduced via palladium- or nickel-catalysed reactions. Here, the heterocycle carries a suitable organometallic leaving group.

In this manner it is possible, according to Scheme 2, to introduce cyanides (nitriles) into the 2-position of the compound 7, which cyanides can then be converted further by known methods, for example into amides, amidoximes, amidines. Amidoximes 11 or 12, for example, can be prepared from the nitriles 10 and hydroxylamine or O-alkylated hydroxylamines, as shown in an exemplary manner in Scheme.

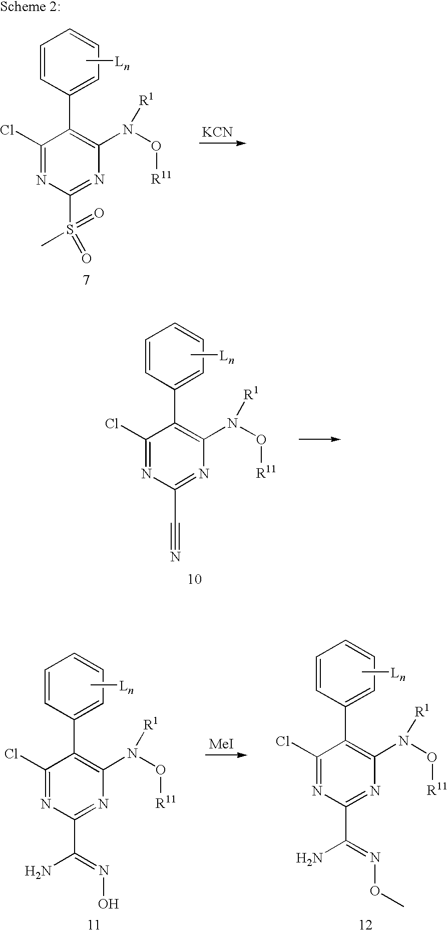

What was said above also applies to the preparation of compounds in which R³ is an alkyl group. An alkyl group (R³) can be introduced via organometallic compounds of the formula (R³)ₙ-M, where M is, for example, magnesium, zinc or lithium, for example at the stage of the compound 5 (see Scheme 1). Here, it is frequently advantageous to use a transition metal catalyst. Particularly successful here was the use of palladium alkyl- and aryl-phosphine complexes. If R³ is a cyano group or an alkoxy substituent, the radical R³ can be introduced by reaction with alkali metal cyanides or alkali metal alkoxides.

An alternative to the preparation of the compounds 6a is the reaction of a dichloropyrimidine with a hydroxylamine derivative 13 and the subsequent halogenation, for example to the iodine derivative 15 (Scheme 3). The iodination can be carried out with the customary reagents such as, for example, iodosuccinimide. Also suitable is I—Cl. Instead of the iodine atom, it is frequently also possible to use bromine for the subsequent reaction. This is then followed by reaction with a benzene derivative 16 where MT is one of the leaving groups customary for transition metal-catalysed C—C bond formations, such as, for example, boron, zinc, tin or magnesium. Here, the free valencies of these metals are preferably taken up by halogen, in the case of boron preferably by hydroxyl or alkoxy. Suitable catalysts are in particular palladium compounds which may carry mono- or bidentate phosphine ligands.

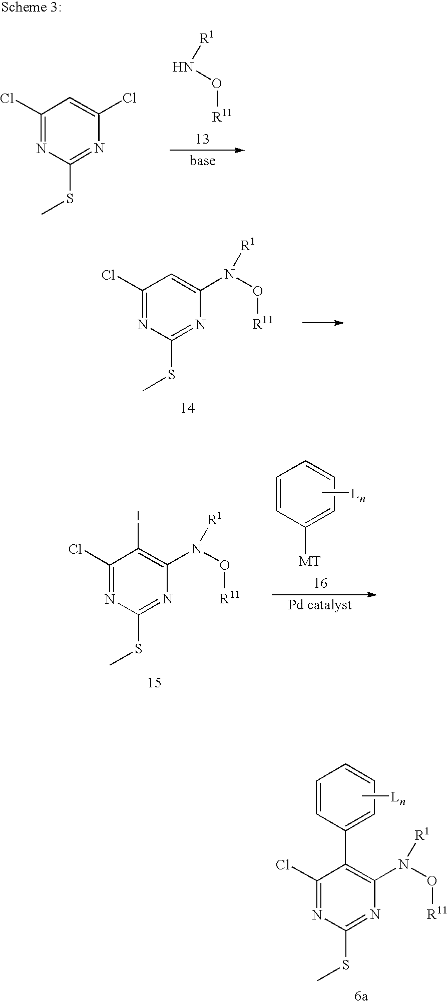

The present invention furthermore provides intermediates for compounds of the formula I according to the invention, in particular intermediates of the formulae 6a′ and 7′

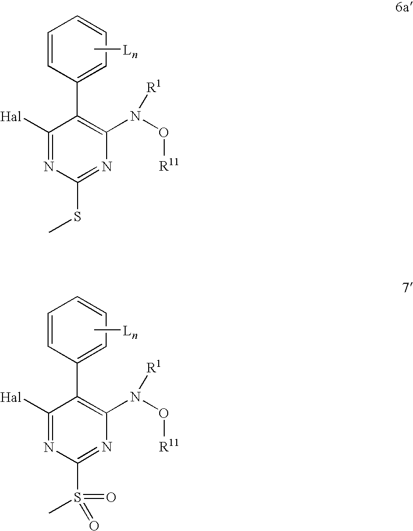

in which Hal is chlorine or bromine and R¹, R¹¹ and Lₙ have the meanings defined for the compounds of the formula I.

In the definitions of the symbols given for the compounds according to the invention, collective terms were used which are generally representative of the following substituents:

halogen: fluorine, chlorine, bromine and iodine;

alkyl and the alkyl moieties of composite groups such as, for example, alkoxy, alkylamino, alkoxycarbonyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4, 6 or 8 carbon atoms (as mentioned above), where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₃)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

hydroxyalkyl: straight-chain or branched alkyl groups having 1 to 2, 4, 6 or 8 carbon atoms (as mentioned above), where one or more hydrogen atoms are replaced by hydroxyl (OH) groups;

alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may furthermore be preferred to use small alkenyl groups, such as (C₂-C₄)-alkenyl, on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: alkenyl as defined above, where some or all of the hydrogen atoms in these groups are replaced by halogen atoms as described above under haloalkyl, in particular fluorine, chlorine or bromine;

alkadienyl: unsaturated straight-chain or branched hydrocarbon radicals having 4 to 6 or 4 to 8 carbon atoms and two double bonds in any position;

alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

haloalkynyl: alkynyl as defined above, where some or all of the hydrogen atoms in these groups are replaced by halogen atoms as described above under haloalkyl, in particular fluorine, chlorine or bromine;

cycloalkyl and also the cycloalkyl moieties in composite groups: mono- or bicyclic saturated hydrocarbon groups having 3 to 6 carbon ring members, for example C₃-C₆-cycloalkyl, such as cyclopropyl, cyclobutyl, cyclopentyl, cyclohexyl;

halocycloalkyl: cycloalkyl as defined above, where some or all of the hydrogen atoms in these groups are replaced by halogen atoms as described above under haloalkyl, in particular fluorine, chlorine or bromine;

cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;

halocycloalkenyl: cycloalkenyl as defined above, where some or all of the hydrogen atoms in these groups are replaced by halogen atoms as described above under haloalkyl, in particular fluorine, chlorine or bromine;

alkoxy: an alkyl group as defined above which is attached via an oxygen, preferably having 1 to 8, more preferably 2 to 6, carbon atoms. According to the invention it may be preferred to use small alkoxy groups, such as (C₁-C₄)-alkoxy, on the other hand, it may also be preferred to use larger alkoxy groups, such as (C₅-C₈)-alkoxy. Examples of preferred alkoxy groups are: methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

haloalkoxy: alkoxy as defined above, where some or all of the hydrogen atoms in these groups are replaced by halogen atoms as described above under haloalkyl, in particular fluorine, chlorine or bromine.

According to the invention it may be preferred to use short-chain haloalkoxy groups, such as (C₁-C₄)-haloalkoxy, on the other hand, it may also be preferred to use relatively long-chain haloalkoxy groups, such as (C₅-C₈)-haloalkoxy.

Examples of preferred haloalkoxy radicals are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;

alkenyloxy: alkenyl as defined above which is attached via an oxygen atom. Preference is given to (C₂-C₈)-alkenyloxy, more preference to (C₃-C₆)-alkenyloxy. According to the invention, it may be preferred to use short-chain alkenyloxy radicals, such as (C₂-C₄)-alkenyloxy, on the other hand, it may also be preferred to use relatively long-chain alkenyloxy groups, such as (C₅-C₈)-alkenyloxy;

alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;

oxyalkylene: alkylene as defined above, preferably with 2 to 4 CH₂ groups, where one valency is attached to the skeleton via an oxygen atom. Examples of preferred oxyalkylene radicals are OCH₂, OCH₂CH₂, OCH₂CH₂CH₂ and OCH₂(CH₂)₂CH₂;

oxyalkyleneoxy: alkylene as defined above, preferably with 1 to 3 CH₂ groups, where both valencies are attached to the skeleton via an oxygen atom. Examples of preferred oxyalkyleneoxy radicals are OCH₂O, OCH₂CH₂O and OCH₂CH₂CH₂O.

Alkylthio: alkyl as defined above which is attached via an S atom.

Alkylsulfinyl: alkyl as defined above which is attached via an SO group.

Alkylsulfonyl: alkyl as defined above which is attached via an S(O)₂ group.

Five- or six-membered saturated, partially unsaturated or aromatic mono- or bicyclic heterocycle which contains one, two, three or four heteroatoms from the group consisting of O, N and S: the heterocycle in question may be attached via a carbon atom or via a nitrogen atom, if present. According to the invention it may be preferred for the heterocycle in question to be attached via carbon; on the other hand, it may also be preferred for the heterocycle to be attached via nitrogen. The heterocycle is in particular:
5- or 6-membered saturated or partially unsaturated heterocyclyl which contains one, two or three nitrogen atoms and/or one oxygen or sulfur atom or one or two oxygen and/or sulfur atoms, where the heterocyclyl may be attached via C or N;
5-membered heteroaryl which contains one, two, three or four nitrogen atoms or one, two or three nitrogen atoms and/or one sulfur- or oxygen atom which may be attached via C or N; or
6-membered heteroaryl which contains one, two, three or four, preferably one, two or three, nitrogen atoms which may be attached via C or N;

5- or 6-membered saturated or partially unsaturated heterocyclyl, which contains one, two or three nitrogen atoms and/or one oxygen or sulfur atom or one or two oxygen and/or sulfur atoms, where the heterocyclyl may be attached via C or N, if present: for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;

5-membered heteroaryl which contains one, two, three or four nitrogen atoms or one, two or three nitrogen atoms and/or one sulfur- or oxygen atom, where the heteroaryl may be attached via C or N, if present: 5-membered heteroaryl groups which, in addition to carbon atoms, may carry one to four nitrogen atoms or one to three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

6-membered heteroaryl, which contains one, two, three or four, preferably one, two or three, nitrogen atoms, where the heteroaryl may be attached via C or N, if present: 6-membered heteroaryl groups which, in addition to carbon atoms, may contain one to four or one to three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The scope of the present invention embraces the (R) and (S) isomers or rotamers and the racemates of compounds according to the invention having chiral centers. The compounds according to the invention may be present in various crystal modifications which may differ in their biological activity. They are likewise provided by the present invention.

With a view to the intended use of the 2-substituted pyrimidines according to the invention, particular preference is given to the following meanings of the substituents, in each case on their own or in combination. The preferred substituents or preferred combinations of substituents apply correspondingly to the precursors of the compounds according to the invention.

Preference is given to compounds according to the invention in which R¹ and R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl, where R¹ and/or R¹¹ may carry one, two, three or four identical or different substituents R², where R² is as defined above.

Here, R² is advantageously halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, where the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; where A, A′ and A″ are as defined above and are preferably hydrogen, C₁-C₆-alkyl, C₂-C₆-alkenyl or phenyl, where the organic radicals may be partially or fully halogenated and/or substituted by C₁-C₄-alkoxy; A and A′ together with the atoms to which they are attached may also be a five- or six-membered saturated heterocycle which contains one or two heteroatoms from the group consisting of O, N and S.

More preferably, R¹ and R¹¹ independently of one another are C₁-C₆-alkyl, C₁-C₆-40 haloalkyl, C₂-C₆-alkenyl, C₂-C₆-haloalkenyl, C₂-C₆-alkynyl, C₂-C₆-haloalkynyl, C₃-C₆-cycloalkyl or C₃-C₆-halocycloalkyl, where R¹ and/or R¹¹ may carry one, two, three or four identical or different substituents R² as defined above.

Also preferably, R¹ and R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl, where R¹ and/or R¹¹ may carry one, two, three or four identical or different substituents R² as defined above.

Furthermore preferably, R¹ and R¹¹ independently of one another are C₁-C₆-alkyl, C₃-C₆-cycloalkyl, (C₁-C₄-alkyl)-C₃-C₆-cycloalkyl, (C₃-C₆-cycloalkyl)-C₁-C₆-alkyl, di-(C₁-C₄-alkyl)-C₃-C₆-cycloalkyl, (C₁-C₄-alkoxy)-C₁-C₆-alkyl, C₂-C₆-alkenyl, C₁-C₆-haloalkyl, C₁-C₆-hydroxyalkyl or C₂-C₆-haloalkenyl. More preferably, R¹ and R¹¹ independently of one another are C₁-C₆-alkyl, C₃-C₆-cycloalkyl, (C₁-C₄-alkyl)-C₃-C₆-cycloalkyl, (C₃-C₆-cycloalkyl)-C₁-C₆-alkyl, di-(C₁-C₄-alkyl)-C₃-C₆-cycloalkyl, (C₁-C₄-alkoxy)-C₁-C₆-alkyl, C₂-C₆-alkenyl, C₁-C₆-haloalkyl or C₂-C₆-haloalkenyl. Preference is furthermore given to compounds according to the invention in which R¹ and R¹¹ independently of one another are cyclopropylmethyl, cyclopentylmethyl, methyl, ethyl, propyl, isopropyl, 1,2-dimethylpropyl, 1,2,2-trimethylpropyl, 1-methyl-2,2,2-trifluoroethyl or 2,2,2-trifluoroethyl.

Also preferably, R¹ and R¹¹ independently of one another are C₁-C₆-alkyl, C₁-C₆-haloalkyl, C₂-C₆-alkenyl, C₂-C₆-alkynyl or C₃-C₆-cycloalkyl.

Especially preferred are compounds according to the invention in which R¹ and R¹¹ independently of one another are C₁-C₆-haloalkyl, C₂-C₆-alkenyl or C₁-C₆-alkyl, branched in the a-position. In addition, preference is given to compounds according to the invention in which R¹ or R¹¹ is C₁-C₄-haloalkyl or C₃-C₆-cycloalkyl-C₁-C₄-alkyl.

In one embodiment of the present invention, R¹ and R¹¹ independently of one another are C₁-C₄-haloalkyl or C₃-C₆-cycloalkyl-C₁-C₄-alkyl or R¹ and R¹¹ together form a five-, six- or seven-membered saturated heterocycle which is optionally substituted by one to four R².

In a further preferred embodiment of the invention, R¹ and R¹¹ together form an optionally substituted five-, six- or seven-membered saturated or unsaturated heterocycle which may contain a further heteroatom from the group consisting of O, N and S as ring member. In one embodiment of the invention, the heterocycle contains no further heteroatoms as ring members.

If an unsaturated heterocycle is formed, this is preferably only partially unsaturated. Particularly preferably, R¹ and R¹¹ form an optionally substituted saturated five-, six- or seven-membered heterocycle, more preferably an optionally substituted saturated five- or six-membered heterocycle.

If the heterocycle formed by R¹ and R¹¹, in particular in the preferred embodiments listed above, is substituted, it contains one, two or three or one, two, three or four independently selected substituents R², as defined above. Here, particularly preferred substituents R² are halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)—C(═O)-A, N(A″)—C(═O)—N(A′)A, or phenyl, where the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; where A, A′ and A″ are as defined above and are preferably independently of one another hydrogen, C₁-C₆-alkyl, C₂-C₆-alkenyl or phenyl, where the organic radicals may be partially or fully halogenated and/or substituted by C₁-C₄-alkoxy; A and A′ together with the atoms to which they are attached may also be a five- or six-membered saturated heterocycle which contains one or two heteroatoms from the group consisting of O, N and S. Particularly preferably, R² is C₁-C₆-alkyl or C₁-C₆-haloalkyl.

Preference is furthermore given to compounds according to the invention in which R³ is halogen, cyano, C₁-C₄-alkyl, C₁-C₄-haloalkyl, C₁-C₄-alkoxy or C₁-C₄-haloalkoxy, preferably halogen, cyano, C₁-C₄-alkyl, C₁-C₄-haloalkyl, C₁-C₄-alkoxy or halomethoxy. Also preferably, R³ is halogen, cyano, C₁-C₄-alkyl, C₁-C₄-haloalkyl or C₁-C₄-alkoxy. With particular preference, R³ is C₁-C₄-alkyl or C₁-C₄-haloalkyl. Furthermore preferably, R³ is halogen, cyano, C₁-C₄-alkyl or C₁-C₄-alkoxy, in particular methyl, cyano, methoxy or halogen, particularly preferably chlorine.

In a preferred embodiment, R⁴ is a five- or six-membered saturated, partially unsaturated or aromatic heterocycle which contains one, two, three or four heteroatoms from the group consisting of O, N and S, where R⁴ may be partially or fully halogenated and/or may carry one, two, three or four identical or different groups R^{u}. Here, the heterocycle may be attached via C or N.

In a further embodiment of the invention, R⁴ is a five- or six-membered saturated, partially unsaturated or aromatic heterocycle which is attached via nitrogen and which contains one, two, three or four heteroatoms from the group consisting of O, N and S, where R⁴ may be partially or fully halogenated and/or may carry one, two, three or four identical or different groups R^{u} or R⁴ is NR^{a}R^{b′}, NR^{a}(C(=Z)R^{b}), NR^{a}(C(=Z)OR^{b}), NR^{a}(C(=Z)-NR^{z}R^{b}), NR^{a}(N═CRCR^{b}), NR^{e}—NR^{z}R^{b} or NR^{z}—OR^{a}.

In a further preferred embodiment of the invention, R⁴ is a five- or six-membered saturated, partially unsaturated or aromatic heterocycle which is attached via carbon and which contains one, two, three or four heteroatoms from the group consisting of O, N and S, where R⁴ may be partially or fully halogenated and/or may carry one, two, three or four identical or different groups R^{u} or R⁴ is cyano, C(=Z)OR^{a}, C(=Z)NR^{z}R^{b}, C(=Z)NR^{a}—NR^{z}R^{b}, C(=Z)R^{a}, CR^{a}R^{b}—OR^{z} or CR^{a}R^{b}—NR^{z}R^{c}.

In a further preferred embodiment of the invention, R⁴ is a five- or six-membered saturated, partially unsaturated or aromatic heterocycle which is attached via nitrogen and which contains one, two, three or four heteroatoms from the group consisting of O, N and S, where R⁴ may be partially or fully halogenated and/or may carry one, two, three or four identical or different groups R^{u} or R⁴ is cyano, C(=Z)OR^{a}, C(=Z)NR^{z}R^{b}, C(=Z)NR^{a}—NR^{z}R^{b}, C(=Z)R^{a}, CR^{a}R^{b}—OR^{z} or CR^{a}R^{b}—NR^{z}R^{c}.

In a further preferred embodiment of the invention, R⁴ is a five- or six-membered saturated or partially unsaturated heterocycle, which may be partially or fully halogenated and/or may carry one, two, three or four identical or different groups R^{u} and may be attached via C or N.

In an even further preferred embodiment of the invention, R⁴ is a five- or six-membered aromatic heterocycle which may be partially or fully halogenated and/or may carry one, two, three or four identical or different groups R^{u} and may be attached via C or N. Here, R⁴ is, according to one embodiment of the invention, an optionally substituted five-membered aromatic heterocycle and according to a further embodiment of the invention, an optionally substituted six-membered aromatic heterocycle, each of which may be attached via C or N.

Preference is furthermore given in particular to compounds according to the invention in which R⁴ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furyl, thienyl, thiazolyl, isothiazolyl, pyridinyl, pyrimidinyl, pyrazinyl, pyridazinyl, 1,2,3-triazinyl, 1,2,4-triazinyl, 1-pyridin(1,2-dihydro)-2-onyl or 1-pyrrolidone. Furthermore preferably, R⁴ is 1-pyrrolidone, imidazolidinone, isoxazolidinone or oxazolidinone, in particular 2-pyrrolidon-1-yl, imidazolidinon-1-yl, isoxazolidin-3-on-2-yl or oxazolin-2-on-3-yl. Here, the heterocycle may in each case be attached to the pyrimidine ring via C or N and is unsubstituted or substituted by one, two or three substituents R^{u}. This preference gives both in combination with the broad definition of R^{u} given in claim 1 and with the following narrower definition of R^{u} compounds which are preferred according to the invention: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N—OA), N(A′)A, N(A′)-C(═O)-A.

Particular preference is given to compounds I in which R⁴ is 1-pyrazolyl, 1-[1,2,4]triazolyl, 2-thiazolyl, 2-pyridinyl, 2-pyrimidinyl, 3-pyridazinyl, 1-pyridin(1,2-dihydro)-2-onyl or 1-pyrrolidonyl, where the heterocycle is unsubstituted or substituted by one, two or three substituents R^{u}. This preference gives both in combination with the broad definition of R^{u} given in claim 1 and with the following narrower definition of R^{u}: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N—OA), N(A′)A, N(A′)-C(═O)-A compounds which are preferred according to the invention.

Preference is furthermore given to compounds according to the invention in which R⁴ is 2-pyridinyl, 3-pyridazinyl, 1-pyridin(1,2-dihydro)-2-onyl or 2-pyrrolidon-1-yl, which radicals are unsubstituted or substituted by one, two or three substituents R^{u}, as defined above, where R^{u} is preferably halogen, cyano, C₁-C₈-alkyl or C₁-C₈-haloalkyl.

Preference is furthermore given to compounds according to the invention in which R⁴ is pyrazolyl or [1,2,4]triazolyl, which radicals are unsubstituted or substituted by one, two or three substituents R^{u}, as defined above, where R^{u} is preferably halogen, cyano, C₁-C₈-alkyl or C₁-C₈-haloalkyl.

Especially preferred are compounds according to the invention in which R⁴ is 2-pyrimidinyl which is unsubstituted or substituted by one, two or three substituents R^{u}, as defined above. This preference gives both in combination with the broad definition of R^{u} given in claim 1 and with the following narrower definition of R^{u}: halogen, cyano, C₁-C₈-alkyl, C₁-C₈-haloalkyl, C₁-C₆-alkoxy, —C(═O)-A, —C(═O)—O-A, —C(═O)—N(A′)A, C(A′)(═N—OA), N(A′)A, N(A′)-C(═O)-A compounds which are preferred according to the invention, where R^{u} is furthermore preferably halogen, cyano, C₁-C₈-alkyl or C₁-C₈-haloalkyl.

In a further preferred embodiment of the invention, R⁴ is cyano, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c}, ON(═CR^{a}R^{b}), O—C(=Z)R^{a}, NR^{a}R^{b′}, NR^{a}(C(=Z)R^{b}), NR^{a}(C(=Z)OR^{b}), NR^{a}(C(=Z)-NR^{z}R^{b}), NR^{a}(N═CRCR^{b}), NR^{a}NR^{z}R^{b}, NR^{z}—OR^{a}.

In a further preferred embodiment of the invention, R⁴ is cyano, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c}, ON(═CR^{a}R^{b}) or O—C(=Z)R^{a}, more preferably cyano, C(=Z)OR^{a}, C(=Z)NR^{z}R^{b}, C(=Z)NR^{a}—NR^{z}R^{b}, C(=Z)R^{a}, CR^{a}R^{b}—OR^{z} or CR^{a}R^{b}—NR^{z}R^{c}.

Preference is furthermore given to compounds according to the invention in which R⁴ is cyano, C(═O)NR^{z}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CR^{c}R^{b}) or NR^{z}—OR^{a}.

Moreover, preference is given to compounds according to the invention in which R⁴ is C(=Z)OR^{a}, C(=Z)NR^{z}R^{b} or C(=Z)R^{a}, where Z is O, NR^{d} or NOR^{d}.

Especially preferred are compounds according to the invention in which R⁴ is C(═O)NH₂ or C(═N—OCH₃)NH₂.

Preference is furthermore given to compounds according to the invention in which R⁴ is C(═NH)NR^{z}R^{b} and R^{z} is a substituent —CO—R^{d} or —COO—R^{d}.

In the compounds according to the invention,

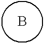

is five- or six-membered heteroaryl which contains 1, 2, 3 or 4, preferably 1, 2 or 3, heteroatoms selected from the group consisting of O, N and S, or phenyl. In one embodiment, the heteroaryl is attached via C, in a further embodiment via N.

In one embodiment,

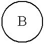

is five- or six-membered heteroaryl which contains 1, 2 or 3 heteroatoms selected from the group consisting of O, N and S, particularly preferably
5-membered heteroaryl which contains one, two or three nitrogen atoms or one or two nitrogen atoms and/or a sulfur or oxygen atom, where the heteroaryl may be attached via C or N: 5-membered heteroaryl groups which, in addition to carbon atoms, may contain one to three nitrogen atoms or one or two nitrogen atoms and/or a sulfur or oxygen atom as ring members, for example furyl, thienyl, pyrrolyl, pyrazolyl, imidazolyl, triazolyl (1,2,3-; 1,2,4-triazolyl), oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which contains one, two or three nitrogen atoms: 6-membered heteroaryl groups which, in addition to carbon atoms, may contain one to three nitrogen atoms as ring members, where the heteroaryl may be attached via C or N: for example pyridine, pyrimidine, pyrazine, pyridazine, 1,2,3-triazine, 1,2,4-triazine, 1,3,5-triazine, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In a further preferred embodiment,

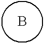

is five-membered heteroaryl which contains 1, 2 or 3 heteroatoms selected from the group consisting of O, N and S. Particular preference is given to pyrazolyl.

In a further preferred embodiment,

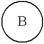

is six-membered heteroaryl which contains 1, 2 or 3 nitrogen atoms. Particular preference is given to pyridyl.

According to a further preferred embodiment,

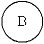

is five-membered heteroaryl which contains 1, 2 or 3 heteroatoms selected from the group consisting of O, N and S, or phenyl, in particular pyrazolyl, pyridyl or phenyl.

In a further preferred embodiment of the invention,

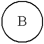

is phenyl.

Especially preferred are 2-substituted pyrimidines according to the invention in which the substituents L (L¹ bis L⁵) independently of one another are as defined below:
L is halogen, cyano, methyl, methoxy, —C(═O)—O-A, —C(═O)—N(A′)A, C(A′)(═N—OA), N(A′)A, N(A′)-C(═O)-A,
   A,A′ independently of one another are hydrogen, C₁-C₆-alkyl, C₂-C₆-alkenyl, C₂-C₆-alkynyl, phenyl, where the organic radicals may be partially or fully halogenated or may be substituted by C₁-C₄-alkoxy, or A and A′ together with the atoms to which they are attached are a five- or six-membered saturated heterocycle which contains one or two heteroatoms from the group consisting of O, N and S;

Moreover, preference is given to pyrimidines according to the invention in which group B substituted by Lₙ is phenyl and is represented by

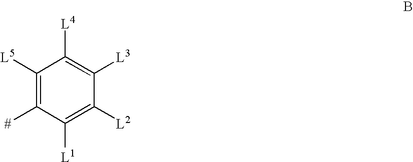

where # is the point of attachment to the pyridine skeleton and
L¹ is fluorine, chlorine, CH₃ or CF₃;
L², L⁴ independently of one another are hydrogen, CH₃ or fluorine;
L³ is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L⁵ is hydrogen, fluorine, chlorine or CH₃.

Preference is furthermore given to 2-substituted pyrimidines of the formula I′,

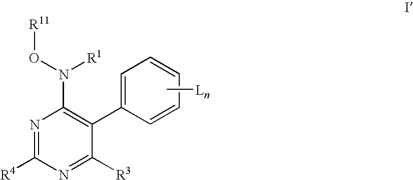

where the indices and substituents are as defined below:
R¹, R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl, where R¹ and R¹¹ together may also form a five-, six- or seven-membered saturated or unsaturated heterocycle; where
   R¹ and/or R¹¹ or a heterocycle formed by R¹ and R¹¹ may carry one, two, three or four identical or different substituents R², where R² is:
   R² halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₃-C₆-cycloalkyloxy, —O(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, where the phenyl moiety may carry one, two or three radicals independently of one another selected from the group consisting of: halogen, C₁-C₆-alkyl, C₂-C₆-alkenyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; where A, A′ and A″ are:
   A, A′, A″ independently of one another hydrogen, C₁-C₆-alkyl, C₂-C₆-alkenyl, phenyl, where the organic radicals may be partially of fully halogenated and/or may be substituted by C₁-C₄-alkoxy; A and A′ together with the atoms to which they are attached may also be a five- or six-membered saturated heterocycle which contains one or two heteroatoms from the group consisting of C, N and S;
R³ is halogen, cyano, C₁-C₄-alkyl, C₁-C₄-alkoxy, C₁-C₄-haloalkoxy or C₁-C₄-haloalkyl, preferably halogen, cyano, C₁-C₄-alkyl, C₁-C₄-alkoxy or C₁-C₄-haloalkyl;
R⁴ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furyl, thienyl, thiazolyl, isothiazolyl, pyridinyl, pyrimidinyl, pyrazinyl, pyridazinyl, 1,2,3-triazinyl, 1,2,4-triazinyl, 1-pyridin(1,2-dihydro)-2-onyl or 1-pyrrolidonyl, where R⁴ is unsubstituted or substituted by one, two or three substituents R^{u}:
   R^{u} is halogen, cyano, C₁-C₈-alkyl, C₁-C₆-alkoxy, —C(═O)-A, —C(═O)—O-A, —C(═O)—N(A′)A, C(A′)(═N—OA), N(A′)A, N(A′)-C(═O)-A; where A,A′ are as defined above;
   or
      cyano, C(═O)NR^{z}R^{b}, C(═O)OR^{a}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CRCR^{b}) or NR^{z}—OR^{a};
L is halogen, cyano, methyl, methoxy, —C(═O)—O-A, —C(═O)—N(A′)A, —C(═S)—N(A′)A, C(A′)(═N—OA), N(A′)A, N(A′)-C(═O)-A; where the aliphatic groups of the radical definitions of L for their part may be partially or fully halogenated;
and
n is 1, 2 or 3, where at least one substituent L on the phenyl ring is located in the ortho-position to the point of attachment to the pyrimidine skeleton.

Preference is also given to 2-substituted pyrimidines of the formula I′ in which the indices and substituents are as defined below:
R¹, R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl, where R¹ and R¹¹ together may also form a five-, six- or seven-membered saturated or unsaturated heterocycle; where
   R¹ and/or R¹¹ or a heterocycle formed by R¹ and R¹¹ may carry one, two, three or four identical or different substituents R², where R² is:
   R² halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, where the phenyl moiety may carry one, two or three radicals independently of one another selected from the group consisting of: halogen, C₁-C₆-alkyl, C₂-C₆-alkenyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; where A, A′ and A″ are:
   A, A′, A″ independently of one another hydrogen or C₁-C₆-alkyl which may be partially or fully halogenated and/or may be substituted by C₁-C₄-alkoxy;
R³ is halogen, cyano, C₁-C₄-alkyl, C₁-C₄-alkoxy, halogenmethoxy or C₁-C₄-haloalkyl;
R⁴ is pyrazolyl, 1,2,3-triazolyl or 1,2,4-triazolyl, where R⁴ is attached to the pyrimidine ring via N and is unsubstituted or substituted by one or two substituents R^{u}:
   R^{u} is halogen, cyano, C₁-C₄-alkyl, C₁-C₄-alkoxy, —C(═O)—O-A, —C(═O)—N(A′)A, C(A′)(═N—OA), N(A′)A, N(A′)-C(═O)-A; where A,A′ are as defined above;
R⁴ may furthermore be:
   C(═O)NR^{z}R^{b}, C(═O)OR^{a}, C(═NOR^{a})NH₂, C(═NOR^{b})R^{a} or NR^{a}(C(═O)OR^{b}); where
      R^{a}, R^{b}, R^{c}, R^{d} independently of one another are hydrogen, C₁-C₆-alkyl, or C₃-C₆-cycloalkyl;
      R^{z} has the same meanings as R^{a} and may additionally be —CO—R^{d} or —COO—R^{d}
L is halogen, cyano, methyl, methoxy, —C(═O)—O-A, —C(═O)—N(A′)A, —C(═S)—N(A′)A, C(A′)(═N—OA), N(A′)A, N(A′)-C(═O)-A; where A and A′ are as defined above;
and
n is 1, 2 or 3, where at least one substituent L on the phenyl ring is located in the ortho-position to the point of attachment to the pyrimidine skeleton.

Preference is also given to 2-substituted pyrimidines of the formula I′

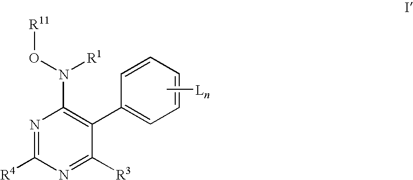

in which the indices and substituents are as defined below:
R¹, R¹¹ independently of one another are hydrogen, C₁-C₆-alkyl, C₂-C₆-alkenyl, C₁-C₆-haloalkyl, C₂-C₆-haloalkenyl, where only one of the two radicals may be hydrogen and R¹ and R¹¹ together may also form a five-, six- or seven-membered saturated heterocycle;
R³ is halogen, cyano, C₁-C₄-alkyl, C₁-C₄-alkoxy or C₁-C₄-haloalkyl;
R⁴ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furyl, thienyl, thiazolyl, isothiazolyl, pyridinyl, pyrimidinyl, pyrazinyl, pyridazinyl, 1,2,3-triazinyl, 1,2,4-triazinyl, 1-pyridin(1,2-dihydro)-2-onyl or 1-pyrrolidonyl (in particular 2-pyrrolidon-1-yl), where R⁴ is unsubstituted or substituted by one, two or three identical or different substituents R^{u}:
   R^{u} is halogen, cyano, C₁-C₈-alkyl, C₁-C₆-alkoxy, —C(═O)-A, —C(═O)—O-A, —C(═O)—N(A′)A, C(A′)(═N—OA), N(A′)A, N(A′)-C(═O)-A,
   R⁴ may furthermore be:
   cyano, C(═O)NR^{z}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}, ON(═CR^{a}R^{b}), NR^{a}(C(═O)R^{b}), NR^{a}(C(═O)OR^{b}), NR^{a}(N═CRCR^{b}) or NR^{z}—OR^{a};
L is halogen, cyano, methyl, methoxy, —C(═O)—O-A, —C(═O)—N(A′)A, C(A′)(═N—OA), N(A′)A, N(A′)-C(═O)-A, where
   A,A′ independently of one another are hydrogen, C₁-C₆-alkyl, C₂-C₆-alkenyl, C₂-C₆-alkynyl, phenyl, where the organic radicals may be partially or fully halogenated and/or substituted by C₁-C₄-alkoxy; A and A′ together with the atoms to which they are attached may also be a five- or six-membered saturated heterocycle which contains one or two heteroatoms from the group consisting of O, N and S;
   where the aliphatic groups of the radical definitions of L for their part may be partially or fully halogenated;
n is 1, 2 or 3, where at least one substituent L on the phenyl ring is located in the ortho-position to the point of attachment to the pyrimidine skeleton.

Particular preference is also given to 2-substituted pyrimidines of the formula I″

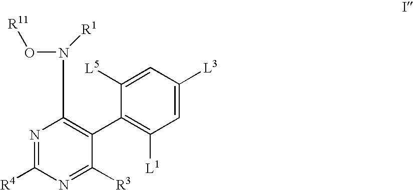

where the substituents are as defined below:
R¹, R¹¹ independently of one another are C₁-C₆-alkyl, C₃-C₆-cycloalkyl-C₁-C₄-alkyl or C₁-C₆-haloalkyl; R¹ and R¹¹ together may also form a five-, six- or seven-membered saturated heterocycle; where the heterocycle may carry one, two, three or four identical or different substituents R², as defined above;
R³ is halogen;
R⁴ is pyrazolyl, 1,2,4-triazolyl, C(═N—OCH₃)NH₂ or CONH₂;
L¹ is chlorine or fluorine;
L³ is fluorine;
L⁵ is hydrogen or fluorine;
   especially preferred are compounds of the formula I″ in which the substituents are as defined below:
R¹, R¹¹ independently of one another are ethyl, propyl, isopropyl, 1,2-dimethylpropyl, 1,2,2-trimethylpropyl, 1-methyl-2,2,2-trifluorethyl or 2,2,2-trifluorethyl;
R³ is fluorine or chlorine;
R⁴ is pyrazolyl, 1,2,4-triazolyl, C(═N—OCH₃)NH₂ or CONH₂;
L¹ is chlorine or fluorine;
L² is fluorine;
L⁵ is hydrogen or fluorine

In particular with respect to their use, particular preference is given to the compounds, shown below, of the formulae Ia, Ib, Ic, Id, Ie, If, Ig, Ih, Ii, Ij, Ik, IL, Im, In, Io, Ip, Iq, Ir, Is, It, Iu, Iv, Iw and Ix where the substituents R¹, R¹¹, R³ and Lₙ have the meanings defined further above for compounds of the formulae I, I′ and/or I″. Especially preferred here are those compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix according to the invention in which the substituents R¹, R¹¹, R³ and/or Lₙ have the preferred meanings given for compounds of the formulae I, I′ and/or I″.

Especially preferred compounds in accordance with the present invention are the compounds I compiled in the tables below (Ia, Ib, Ic, Id, Ie, If, Ig, Ih, Ii, Ij, Ik, IL, Im, In, Io, Ip, Iq, Ir, Is, It, Iu, Iv, Iw and Ix). Here, the groups mentioned in the tables for a substituent are furthermore, independently of the combination in which they are mentioned, a particularly preferred embodiment of the substituent in question.

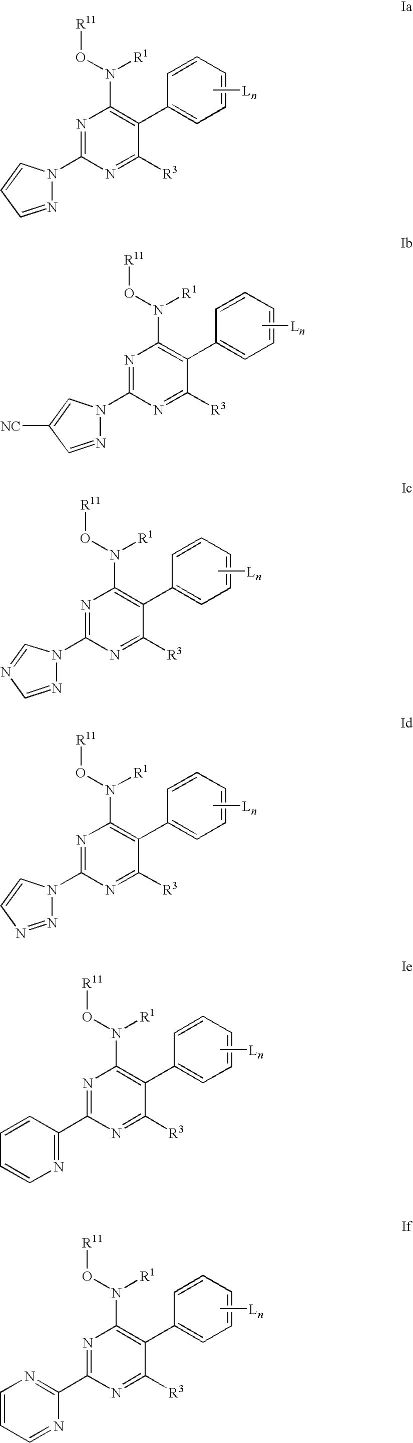

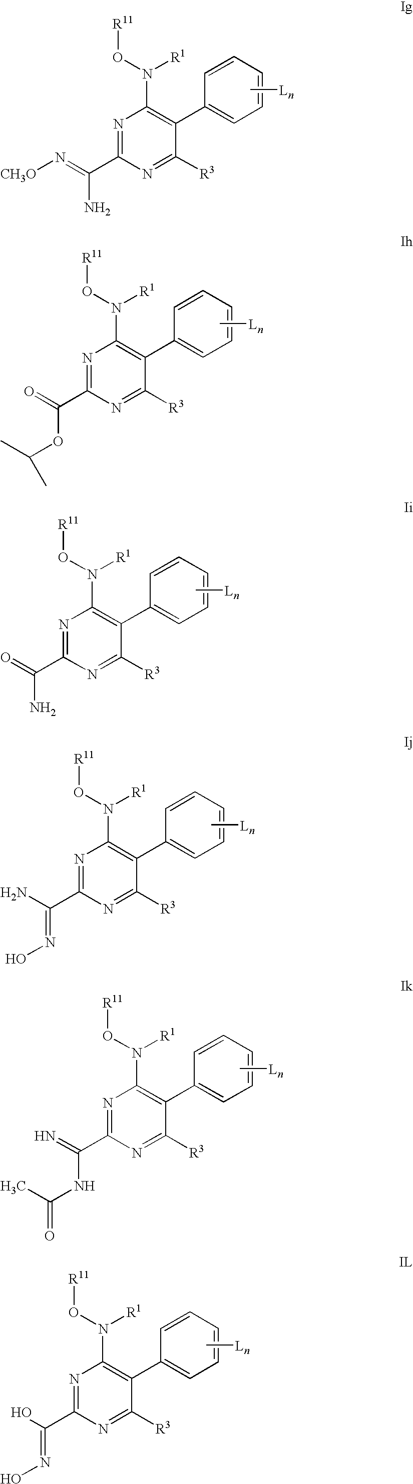

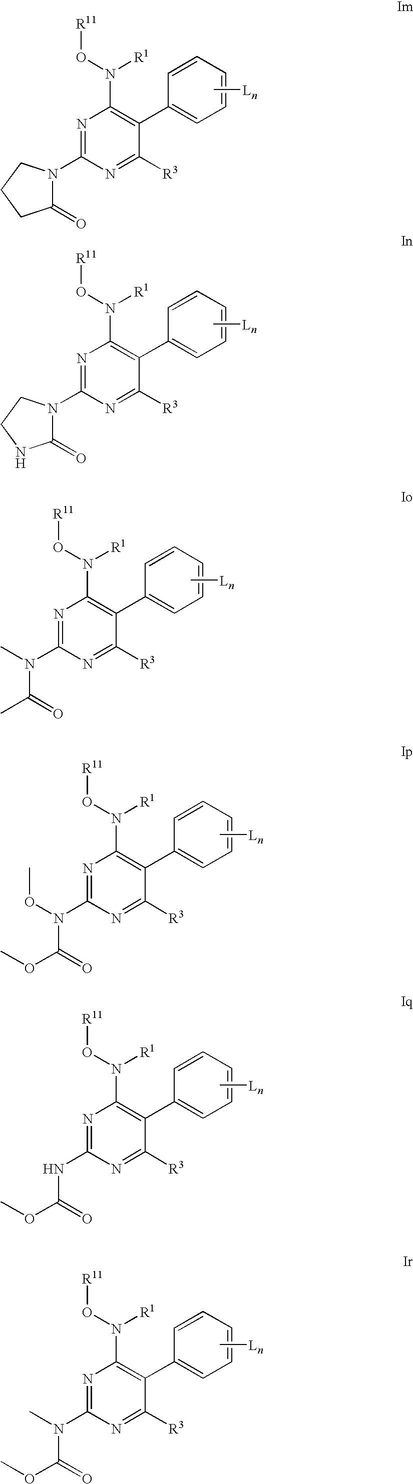

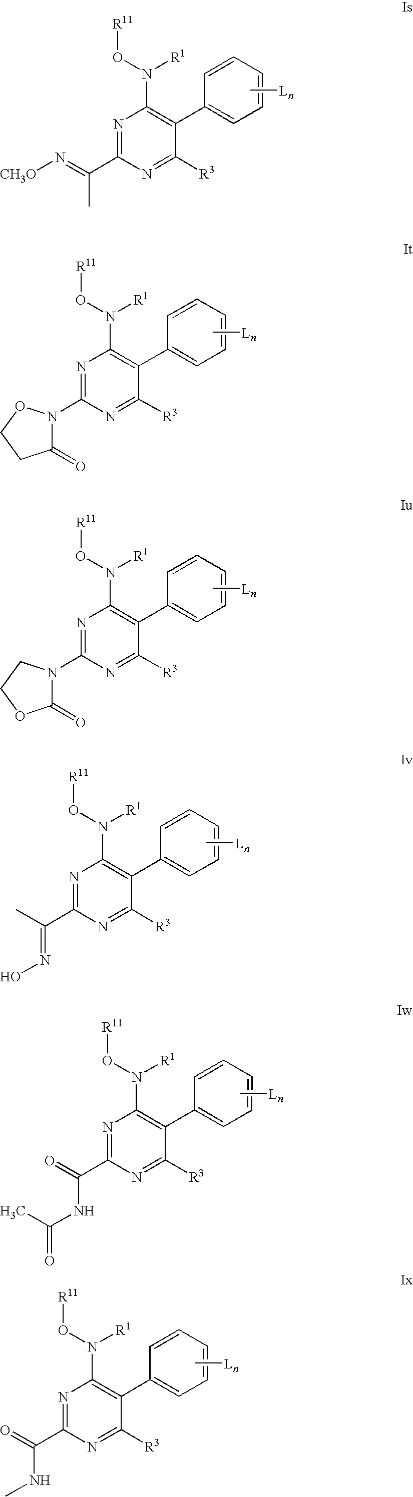

### Table 1

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-chloro, R³ is methyl and R¹ and R¹¹ for a compound corresponds in each case to one row of Table A

### Table 2

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro, R³ is methyl and R¹ and R¹¹ for a compound corresponds in each case to one row of Table A

### Table 3

Compounds of the formulae Ia, Ib, Ic, Id, Ie, If, Ig, Ih, ii, Ij, Ik, IL, Im, In, Io, Ip, Iq, Ir, Is, It, Iu, Iv, Iw and Ix in which Lₙ is 2,6-dichloro, R³ is methyl and R¹ and R¹¹ for a compound corresponds in each case to one row of Table A

### Table 4

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-methyl, R³ is methyl and R¹ and R¹¹ for a compound corresponds in each case to one row of Table A

### Table 5

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trifluoro, R³ is methyl and R¹ and R¹¹ for a compound corresponds in each case to one row of Table A

### Table 6

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-fluoro, R³ is methyl and R¹ and R¹¹ for a compound corresponds in each case to one row of Table A

### Table 7

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxycarbonyl, R³ is methyl and R¹ and R¹¹ for a compound corresponds in each case to one row of Table A

### Table 8

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-CN, R³ is methyl and R¹ and R¹¹ for a compound corresponds in each case to one row of Table A

### Table 9

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,5-trifluoro, R³ is methyl and R¹ and R¹¹ for a compound corresponds in each case to one row of Table A

### Table 10

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dichloro, R³ is methyl and R¹ and R¹¹ for a compound corresponds in each case to one row of Table A

### Table 11

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro, R³ is methyl and R¹ and R¹¹ for a compound corresponds in each case to one row of Table A

### Table 12

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro, R³ is methyl and R¹ and R¹¹ for a compound corresponds in each case to one row of Table A

### Table 13

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-difluoro, R³ is methyl and R¹ and R¹¹ for a compound corresponds in each case to one row of Table A

### Table 14

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-chloro, R³ is methyl and R¹ and R¹¹ for a compound corresponds in each case to one row of Table A

### Table 15

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-4-fluoro, R³ is methyl and R¹ and R¹¹ for a compound corresponds in each case to one row of Table A

### Table 16

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-5-fluoro, R³ is methyl and R¹ and R¹¹ for a compound corresponds in each case to one row of Table A

### Table 17

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3-difluoro, R³ is methyl and R¹ and R¹¹ for a compound corresponds in each case to one row of Table A

### Table 18

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-difluoro, R³ is methyl and R¹ and R¹¹ for a compound corresponds in each case to one row of Table A

### Table 19

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3,4-trifluoro, R³ is methyl and R¹ and R¹¹ for a compound corresponds in each case to one row of Table A

### Table 20

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl, R³ is methyl and R¹ and R¹¹ for a compound corresponds in each case to one row of Table A

### Table 21

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dimethyl, R³ is methyl and R¹ and R¹¹ for a compound corresponds in each case to one row of Table A

### Table 22

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and ix in which Lₙ is 2-methyl-4-chloro, R³ is methyl and R¹ and R¹¹ for a compound corresponds in each case to one row of Table A

### Table 23

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-methyl, R³ is methyl and R¹ and R¹¹ for a compound corresponds in each case to one row of Table A

### Table 24

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dimethyl, R³ is methyl and R¹ and R¹¹ for a compound corresponds in each case to one row of Table A

### Table 25

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trimethyl, R³ is methyl and R¹ and R¹¹ for a compound corresponds in each case to one row of Table A

### Table 26

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-cyano, R³ is methyl and R¹ and R¹¹ for a compound corresponds in each case to one row of Table A

### Table 27

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methyl, R³ is methyl and R¹ and R¹¹ for a compound corresponds in each case to one row of Table A

### Table 28

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methoxycarbonyl, R³ is methyl and R¹ and R¹¹ for a compound corresponds in each case to one row of Table A

### Table 29

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxy, R³ is methyl and R¹ and R¹¹ for a compound corresponds in each case to one row of Table A

### Table 30

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methyl, R³ is methyl and R¹ and R¹¹ for a compound corresponds in each case to one row of Table A

### Table 31

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxycarbonyl, R³ is methyl and R¹ and R¹¹ for a compound corresponds in each case to one row of Table A

### Table 32

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-bromo, R³ is methyl and R¹ and R¹¹ for a compound corresponds in each case to one row of Table A

### Table 33

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-cyano, R³ is methyl and R¹ and R¹¹ for a compound corresponds in each case to one row of Table A

### Table 34

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro,4-methoxy, R³ is methyl and R¹ and R¹¹ for a compound corresponds in each case to one row of Table A

### Table 35

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,3-methyl, R³ is methyl and R¹ and R¹¹ for a compound corresponds in each case to one row of Table A

### Table 36

Compounds of the formulae Ia, Ib, Ic, Id, Ie, If, Ig, Ih, ii, Ij, Ik, IL, Im, In, Io, Ip, Iq, Ir, Is, It, Iu, Iv, Iw and Ix in which Lₙ is 2,5-dimethyl, R³ is methyl and R¹ and R¹¹ for a compound corresponds in each case to one row of Table A

### Table 37

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-cyano, R³ is methyl and R¹ and R¹¹ for a compound corresponds in each case to one row of Table A

### Table 38

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-bromo, R³ is methyl and R¹ and R¹¹ for a compound corresponds in each case to one row of Table A

### Table 39

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,5-fluoro, R³ is methyl and R¹ and R¹¹ for a compound corresponds in each case to one row of Table A

### Table 40

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxy, R³ is methyl and R¹ and R¹¹ for a compound corresponds in each case to one row of Table A

### Table 41

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxycarbonyl, R³ is methyl and R¹ and R¹¹ for a compound corresponds in each case to one row of Table A

### Table 42

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl,4-bromo, R³ is methyl and R¹ and R¹¹ for a compound corresponds in each case to one row of Table A

### Table 43

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-bromo, R³ is methyl and R¹ and R¹¹ for a compound corresponds in each case to one row of Table A

### Table 44

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxy, R³ is methyl and R¹ and R¹¹ for a compound corresponds in each case to one row of Table A

### Table 45

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,5-methyl, R³ is methyl and R¹ and R¹¹ for a compound corresponds in each case to one row of Table A

### Table 46

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is pentafluoro, R³ is methyl and R¹ and R¹¹ for a compound corresponds in each case to one row of Table A

### Table 47

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-chloro, R³ is chlorine and R¹ and R¹¹ for a compound corresponds in each case to one row of Table A

### Table 48

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro, R³ is chlorine and R¹ and R¹¹ for a compound corresponds in each case to one row of Table A

### Table 49

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dichloro, R³ is chlorine and R¹ and R¹¹ for a compound corresponds in each case to one row of Table A

### Table 50

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-methyl, R³ is chlorine and R¹ and R¹¹ for a compound corresponds in each case to one row of Table A

### Table 51

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trifluoro, R³ is chlorine and R¹ and R¹¹ for a compound corresponds in each case to one row of Table A

### Table 52

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-fluoro, R³ is chlorine and R¹ and R¹¹ for a compound corresponds in each case to one row of Table A

### Table 53

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxycarbonyl, R³ is chlorine and R¹ and R¹¹ for a compound corresponds in each case to one row of Table A

### Table 54

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-CN, R³ is chlorine and R¹ and R¹¹ for a compound corresponds in each case to one row of Table A

### Table 55

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,5-trifluoro, R³ is chlorine and R¹ and R¹¹ for a compound corresponds in each case to one row of Table A

### Table 56

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dichloro, R³ is chlorine and R¹ and R¹¹ for a compound corresponds in each case to one row of Table A

### Table 57

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro, R³ is chlorine and R¹ and R¹¹ for a compound corresponds in each case to one row of Table A

### Table 58

Compounds of the formulae Ia, Ib, Ic, Id, Ie, If, Ig, Ih, ii, Ij, Ik, IL, Im, In, Io, Ip, Iq, Ir, Is, It, Iu, Iv, Iw and Ix in which Lₙ is 2-fluoro, R³ is chlorine and R¹ and R¹¹ for a compound corresponds in each case to one row of Table A

### Table 59

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-difluoro, R³ is chlorine and R¹ and R¹¹ for a compound corresponds in each case to one row of Table A

### Table 60

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-chloro, R³ is chlorine and R¹ and R¹¹ for a compound corresponds in each case to one row of Table A

### Table 61

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-4-fluoro, R³ is chlorine and R¹ and R¹¹ for a compound corresponds in each case to one row of Table A

### Table 62

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-5-fluoro, R³ is chlorine and R¹ and R¹¹ for a compound corresponds in each case to one row of Table A

### Table 63

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3-difluoro, R³ is chlorine and R¹ and R¹¹ for a compound corresponds in each case to one row of Table A

### Table 64

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-difluoro, R³ is chlorine and R¹ and R¹¹ for a compound corresponds in each case to one row of Table A

### Table 65

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3,4-trifluoro, R³ is chlorine and R¹ and R¹¹ for a compound corresponds in each case to one row of Table A

### Table 66

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl, R³ is chlorine and R¹ and R¹¹ for a compound corresponds in each case to one row of Table A

### Table 67

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dimethyl, R³ is chlorine and R¹ and R¹¹ for a compound corresponds in each case to one row of Table A

### Table 68

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl-4-chloro, R³ is chlorine and R¹ and R¹¹ for a compound corresponds in each case to one row of Table A

### Table 69

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-methyl, R³ is chlorine and R¹ and R¹¹ for a compound corresponds in each case to one row of Table A

### Table 70

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dimethyl, R³ is chlorine and R¹ and R¹¹ for a compound corresponds in each case to one row of Table A

### Table 71

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trimethyl, R³ is chlorine and R¹ and R¹¹ for a compound corresponds in each case to one row of Table A

### Table 72

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-cyano, R³ is chlorine and R¹ and R¹¹ for a compound corresponds in each case to one row of Table A

### Table 73

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methyl, R³ is chlorine and R¹ and R¹¹ for a compound corresponds in each case to one row of Table A

### Table 74

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methoxycarbonyl, R³ is chlorine and R¹ and R¹¹ for a compound corresponds in each case to one row of Table A

### Table 75

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxy, R³ is chlorine and R¹ and R¹¹ for a compound corresponds in each case to one row of Table A

### Table 76

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methyl, R³ is chlorine and R¹ and R¹¹ for a compound corresponds in each case to one row of Table A

### Table 77

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxycarbonyl, R³ is chlorine and R¹ and R¹¹ for a compound corresponds in each case to one row of Table A

### Table 78

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-bromo, R³ is chlorine and R¹ and R¹¹ for a compound corresponds in each case to one row of Table A

### Table 79

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-cyano, R³ is chlorine and R¹ and R¹¹ for a compound corresponds in each case to one row of Table A

### Table 80

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro,4-methoxy, R³ is chlorine and R¹ and R¹¹ for a compound corresponds in each case to one row of Table A

### Table 81

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,3-methyl, R³ is chlorine and R¹ and R¹¹ for a compound corresponds in each case to one row of Table A

### Table 82

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl, R³ is chlorine and R¹ and R¹¹ for a compound corresponds in each case to one row of Table A

### Table 83

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-cyano, R³ is chlorine and R¹ and R¹¹ for a compound corresponds in each case to one row of Table A

### Table 84

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-bromo, R³ is chlorine and R¹ and R¹¹ for a compound corresponds in each case to one row of Table A

### Table 85

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,5-fluoro, R³ is chlorine and R¹ and R¹¹ for a compound corresponds in each case to one row of Table A

### Table 86

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxy, R³ is chlorine and R¹ and R¹¹ for a compound corresponds in each case to one row of Table A

### Table 87

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxycarbonyl, R³ is chlorine and R¹ and R¹¹ for a compound corresponds in each case to one row of Table A

### Table 88

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl,4-bromo, R³ is chlorine and R¹ and R¹¹ for a compound corresponds in each case to one row of Table A

### Table 89

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-bromo, R³ is chlorine and R¹ and R¹¹ for a compound corresponds in each case to one row of Table A

### Table 90

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxy, R³ is chlorine and R¹ and R¹¹ for a compound corresponds in each case to one row of Table A

### Table 91

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,5-methyl, R³ is chlorine and R¹ and R¹¹ for a compound corresponds in each case to one row of Table A

### Table 92

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is pentafluoro, R³ is chlorine and R¹ and R¹¹ for a compound corresponds in each case to one row of Table A

### Table 93

Compounds of the formulae Ia, Ib, Ic, Id, Ie, If, Ig, Ih, ii, Ij, Ik, IL, Im, In, Io, Ip, Iq, Ir, Is, It, Iu, Iv, Iw and Ix in which Lₙ is 2-fluoro,6-chloro, R³ is methoxy and R¹ and R¹¹ for a compound corresponds in each case to one row of Table A

### Table 94

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro, R³ is methoxy and R¹ and R¹¹ for a compound corresponds in each case to one row of Table A

### Table 95

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dichloro, R³ is methoxy and R¹ and R¹¹ for a compound corresponds in each case to one row of Table A

### Table 96

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-methyl, R³ is methoxy and R¹ and R¹¹ for a compound corresponds in each case to one row of Table A

### Table 97

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trifluoro, R³ is methoxy and R¹ and R¹¹ for a compound corresponds in each case to one row of Table A

### Table 98

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-fluoro, R³ is methoxy and R¹ and R¹¹ for a compound corresponds in each case to one row of Table A

### Table 99

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxycarbonyl, R³ is methoxy and R¹ and R¹¹ for a compound corresponds in each case to one row of Table A

### Table 100

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-CN, R³ is methoxy and R¹ and R¹¹ for a compound corresponds in each case to one row of Table A

### Table 101

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,5-trifluoro, R³ is methoxy and R¹ and R¹¹ for a compound corresponds in each case to one row of Table A

### Table 102

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and ix in which Lₙ is 2,4-dichloro, R³ is methoxy and R¹ and R¹¹ for a compound corresponds in each case to one row of Table A

### Table 103

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro, R³ is methoxy and R¹ and R¹¹ for a compound corresponds in each case to one row of Table A

### Table 104

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro, R³ is methoxy and R¹ and R¹¹ for a compound corresponds in each case to one row of Table A

### Table 105

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-difluoro, R³ is methoxy and R¹ and R¹¹ for a compound corresponds in each case to one row of Table A

### Table 106

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-chloro, R³ is methoxy and R¹ and R¹¹ for a compound corresponds in each case to one row of Table A

### Table 107

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-4-fluoro, R³ is methoxy and R¹ and R¹¹ for a compound corresponds in each case to one row of Table A

### Table 108

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-5-fluoro, R³ is methoxy and R¹ and R¹¹ for a compound corresponds in each case to one row of Table A

### Table 109

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3-difluoro, R³ is methoxy and R¹ and R¹¹ for a compound corresponds in each case to one row of Table A

### Table 110

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-difluoro, R³ is methoxy and R¹ and R¹¹ for a compound corresponds in each case to one row of Table A

### Table 111

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3,4-trifluoro, R³ is methoxy and R¹ and R¹¹ for a compound corresponds in each case to one row of Table A

### Table 112

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl, R³ is methoxy and R¹ and R¹¹ for a compound corresponds in each case to one row of Table A

### Table 113

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dimethyl, R³ is methoxy and R¹ and R¹¹ for a compound corresponds in each case to one row of Table A

### Table 114

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl-4-chloro, R³ is methoxy and R¹ and R¹¹ for a compound corresponds in each case to one row of Table A

### Table 115

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-methyl, R³ is methoxy and R¹ and R¹¹ for a compound corresponds in each case to one row of Table A

### Table 116

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dimethyl, R³ is methoxy and R¹ and R¹¹ for a compound corresponds in each case to one row of Table A

### Table 117

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trimethyl, R³ is methoxy and R¹ and R¹¹ for a compound corresponds in each case to one row of Table A

### Table 118

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-cyano, R³ is methoxy and R¹ and R¹¹ for a compound corresponds in each case to one row of Table A

### Table 119

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methyl, R³ is methoxy and R¹ and R¹¹ for a compound corresponds in each case to one row of Table A

### Table 120

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methoxycarbonyl, R³ is methoxy and R¹ and R¹¹ for a compound corresponds in each case to one row of Table A

### Table 121

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxy, R³ is methoxy and R¹ and R¹¹ for a compound corresponds in each case to one row of Table A

### Table 122

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methyl, R³ is methoxy and R¹ and R¹¹ for a compound corresponds in each case to one row of Table A

### Table 123

Compounds of the formulae Ia, Ib, Ic, Id, Ie, If, Ig, Ih, Ii, Ij, Ik, IL, Im, In, Io, Ip, Iq, Ir, Is, it, Iu, Iv, Iw and Ix in which Lₙ is 2-chloro,4-methoxycarbonyl, R³ is methoxy and R¹ and R¹¹ for a compound corresponds in each case to one row of Table A

### Table 124

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-bromo, R³ is methoxy and R¹ and R¹¹ for a compound corresponds in each case to one row of Table A

### Table 125

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-cyano, R³ is methoxy and R¹ and R¹¹ for a compound corresponds in each case to one row of Table A

### Table 126

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro,4-methoxy, R³ is methoxy and R¹ and R¹¹ for a compound corresponds in each case to one row of Table A

### Table 127

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,3-methyl, R³ is methoxy and R¹ and R¹¹ for a compound corresponds in each case to one row of Table A

### Table 128

Compounds of the formulae Ia, Ib, Ic, Id, Ie, If, Ig, Ih, Ii, Ij, Ik, IL, Im, In, Io, Ip, Iq, Ir, Is, It, IU, Iv, Iw and Ix in which Lₙ is 2,5-dimethyl, R³ is methoxy and R¹ and R¹¹ for a compound corresponds in each case to one row of Table A

### Table 129

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-cyano, R³ is methoxy and R¹ and R¹¹ for a compound corresponds in each case to one row of Table A

### Table 130

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-bromo, R³ is methoxy and R¹ and R¹¹ for a compound corresponds in each case to one row of Table A

### Table 131

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,5-fluoro, R³ is methoxy and R¹ and R¹¹ for a compound corresponds in each case to one row of Table A

### Table 132

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxy, R³ is methoxy and R¹ and R¹¹ for a compound corresponds in each case to one row of Table A

### Table 133

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxycarbonyl, R³ is methoxy and R¹ and R¹¹ for a compound corresponds in each case to one row of Table A

### Table 134

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl,4-bromo, R³ is methoxy and R¹ and R¹¹ for a compound corresponds in each case to one row of Table A

### Table 135

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-bromo, R³ is methoxy and R¹ and R¹¹ for a compound corresponds in each case to one row of Table A

### Table 136

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxy, R³ is methoxy and R¹ and R¹¹ for a compound corresponds in each case to one row of Table A

### Table 137

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,5-methyl, R³ is methoxy and R¹ and R¹¹ for a compound corresponds in each case to one row of Table A

### Table 138

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is pentafluoro, R³ is methoxy and R¹ and R¹¹ for a compound corresponds in each case to one row of Table A

### Table 139

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-chloro, R³ is cyano and R¹ and R¹¹ for a compound corresponds in each case to one row of Table A

### Table 140

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro, R³ is cyano and R¹ and R¹¹ for a compound corresponds in each case to one row of Table A

### Table 141

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dichloro, R³ is cyano and R¹ and R¹¹ for a compound corresponds in each case to one row of Table A

### Table 142

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,6-methyl, R³ is cyano and R¹ and R¹¹ for a compound corresponds in each case to one row of Table A

### Table 143

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trifluoro, R³ is cyano and R¹ and R¹¹ for a compound corresponds in each case to one row of Table A

### Table 144

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-fluoro, R³ is cyano and R¹ and R¹¹ for a compound corresponds in each case to one row of Table A

### Table 145

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxycarbonyl, R³ is cyano and R¹ and R¹¹ for a compound corresponds in each case to one row of Table A

### Table 146

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-CN, R³ is cyano and R¹ and R¹¹ for a compound corresponds in each case to one row of Table A

### Table 147

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,5-trifluoro, R³ is cyano and R¹ and R¹¹ for a compound corresponds in each case to one row of Table A

### Table 148

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dichloro, R³ is cyano and R¹ and R¹¹ for a compound corresponds in each case to one row of Table A

### Table 149

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro, R³ is cyano and R¹ and R¹¹ for a compound corresponds in each case to one row of Table A

### Table 150

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro, R³ is cyano and R¹ and R¹¹ for a compound corresponds in each case to one row of Table A

### Table 151

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-difluoro, R³ is cyano and R¹ and R¹¹ for a compound corresponds in each case to one row of Table A

### Table 152

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-chloro, R³ is cyano and R¹ and R¹¹ for a compound corresponds in each case to one row of Table A

### Table 153

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-4-fluoro, R³ is cyano and R¹ and R¹¹ for a compound corresponds in each case to one row of Table A

### Table 154

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro-5-fluoro, R³ is cyano and R¹ and R¹¹ for a compound corresponds in each case to one row of Table A

### Table 155

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3-difluoro, R³ is cyano and R¹ and R¹¹ for a compound corresponds in each case to one row of Table A

### Table 156

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-difluoro, R³ is cyano and R¹ and R¹¹ for a compound corresponds in each case to one row of Table A

### Table 157

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,3,4-trifluoro, R³ is cyano and R¹ and R¹¹ for a compound corresponds in each case to one row of Table A

### Table 158

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl, R³ is cyano and R¹ and R¹¹ for a compound corresponds in each case to one row of Table A

### Table 159

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4-dimethyl, R³ is cyano and R¹ and R¹¹ for a compound corresponds in each case to one row of Table A

### Table 160

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl-4-chloro, R³ is cyano and R¹ and R¹¹ for a compound corresponds in each case to one row of Table A

### Table 161

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro-4-methyl, R³ is cyano and R¹ and R¹¹ for a compound corresponds in each case to one row of Table A

### Table 162

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-dimethyl, R³ is cyano and R¹ and R¹¹ for a compound corresponds in each case to one row of Table A

### Table 163

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,4,6-trimethyl, R³ is cyano and R¹ and R¹¹ for a compound corresponds in each case to one row of Table A

### Table 164

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-cyano, R³ is cyano and R¹ and R¹¹ for a compound corresponds in each case to one row of Table A

### Table 165

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methyl, R³ is cyano and R¹ and R¹¹ for a compound corresponds in each case to one row of Table A

### Table 166

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro-4-methoxycarbonyl, R³ is cyano and R¹ and R¹¹ for a compound corresponds in each case to one row of Table A

### Table 167

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxy, R³ is cyano and R¹ and R¹¹ for a compound corresponds in each case to one row of Table A

### Table 168

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methyl, R³ is cyano and R¹ and R¹¹ for a compound corresponds in each case to one row of Table A

### Table 169

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-methoxycarbonyl, R³ is cyano and R¹ and R¹¹ for a compound corresponds in each case to one row of Table A

### Table 170

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-bromo, R³ is cyano and R¹ and R¹¹ for a compound corresponds in each case to one row of Table A

### Table 171

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-chloro,4-cyano, R³ is cyano and R¹ and R¹¹ for a compound corresponds in each case to one row of Table A

### Table 172

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,6-difluoro,4-methoxy, R³ is cyano and R¹ and R¹¹ for a compound corresponds in each case to one row of Table A

### Table 173

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,3-methyl, R³ is cyano and R¹ and R¹¹ for a compound corresponds in each case to one row of Table A

### Table 174

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl, R³ is cyano and R¹ and R¹¹ for a compound corresponds in each case to one row of Table A

### Table 175

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-cyano, R³ is cyano and R¹ and R¹¹ for a compound corresponds in each case to one row of Table A

### Table 176

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-bromo, R³ is cyano and R¹ and R¹¹ for a compound corresponds in each case to one row of Table A

### Table 177

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,5-fluoro, R³ is cyano and R¹ and R¹¹ for a compound corresponds in each case to one row of Table A

### Table 178

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxy, R³ is cyano and R¹ and R¹¹ for a compound corresponds in each case to one row of Table A

### Table 179

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-methyl,4-methoxycarbonyl, R³ is cyano and R¹ and R¹¹ for a compound corresponds in each case to one row of Table A

### Table 180

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2,5-dimethyl,4-bromo, R³ is cyano and R¹ and R¹¹ for a compound corresponds in each case to one row of Table A

### Table 181

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-bromo, R³ is cyano and R¹ and R¹¹ for a compound corresponds in each case to one row of Table A

### Table 182

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,4-methoxy, R³ is cyano and R¹ and R¹¹ for a compound corresponds in each case to one row of Table A

### Table 183

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is 2-fluoro,5-methyl, R³ is cyano and R¹ and R¹¹ for a compound corresponds in each case to one row of Table A

### Table 184

Compounds of the formulae Ia, Ib, Ic, Id, Ie, If, Ig, Ih, Ii, Ij, Ik, IL, Im, In, Io, Ip, Iq, Ir, Is, It, Iu, Iv, Iw and Ix in which Lₙ is pentafluoro, R³ is cyano and R¹ and R¹¹ for a compound corresponds in each case to one row of Table A

TABLE A

| No. | R¹ | R¹¹ |
|---|---|---|
| A-1 | CH₂CH₃ | CH₃ |
| A-2 | CH₂CH₂CH₃ | CH₃ |
| A-3 | CH₂CH₂F | CH₃ |
| A-4 | CH₃ | CH₃ |
| A-5 | H | CH₃ |
| A-6 | CH(CH₃)₂ | CH₃ |
| A-7 | CH₂C(CH₃)₃ | CH₃ |
| A-8 | CH₂CH(CH₃)₂ | CH₃ |
| A-9 | (±)CH(CH₂CH₃)CH₃ | CH₃ |
| A-10 | (R)CH(CH₂CH₃)CH₃ | CH₃ |
| A-11 | (S)CH(CH₂CH₃)CH₃ | CH₃ |
| A-12 | (±)CH(CH₃)—CH(CH₃)₂ | CH₃ |
| A-13 | (R)CH(CH₃)—CH(CH₃)₂ | CH₃ |
| A-14 | (S)CH(CH₃)—CH(CH₃)₂ | CH₃ |
| A-15 | (±)CH(CH₃)—C(CH₃)₃ | CH₃ |
| A-16 | (R)CH(CH₃)—C(CH₃)₃ | CH₃ |
| A-17 | (S)CH(CH₃)—C(CH₃)₃ | CH₃ |
| A-18 | (±)CH(CH₃)—CF₃ | CH₃ |
| A-19 | (R)CH(CH₃)—CF₃ | CH₃ |
| A-20 | (S)CH(CH₃)—CF₃ | CH₃ |
| A-21 | (±)CH(CH₃)—CCl₃ | CH₃ |
| A-22 | (R)CH(CH₃)—CCl₃ | CH₃ |
| A-23 | (S)CH(CH₃)—CCl₃ | CH₃ |
| A-24 | CH₂C(CH₃)═CH₂ | CH₃ |
| A-25 | cyclopentyl | CH₃ |
| A-26 | cyclohexyl | CH₃ |
| A-27 | (CH₂)₃CH₃ | CH₃ |
| A-28 | C(CH₃)₃ | CH₃ |
| A-29 | (CH₂)₄CH₃ | CH₃ |
| A-30 | CH(CH₂CH₃)₂ | CH₃ |
| A-31 | CH₂CH₂CH(CH₃)₂ | CH₃ |
| A-32 | (±)CH(CH₃)(CH₂)₂CH₃ | CH₃ |
| A-33 | (R)CH(CH₃)(CH₂)₂CH₃ | CH₃ |
| A-34 | (S)CH(CH₃)(CH₂)₂CH₃ | CH₃ |
| A-35 | (±)CH₂CH(CH₃)CH₂CH₃ | CH₃ |
| A-36 | (R)CH₂CH(CH₃)CH₂CH₃ | CH₃ |
| A-37 | (S)CH₂CH(CH₃)CH₂CH₃ | CH₃ |
| A-38 | (±)CH(CH₃)CH(CH₃)₂ | CH₃ |
| A-39 | (R)CH(CH₃)CH(CH₃)₂ | CH₃ |
| A-40 | (S)CH(CH₃)CH(CH₃)₂ | CH₃ |
| A-41 | (CH₂)₅CH₃ | CH₃ |
| A-42 | (±,±)CH(CH₃)CH(CH₃)CH₂CH₃ | CH₃ |
| A-43 | (±,R)CH(CH₃)CH(CH₃)CH₂CH₃ | CH₃ |
| A-44 | (±,S)CH(CH₃)CH(CH₃)CH₂CH₃ | CH₃ |
| A-45 | (±)CH₂CH(CH₃)CF₃ | CH₃ |
| A-46 | (R)CH₂CH(CH₃)CF₃ | CH₃ |
| A-47 | (S)CH₂CH(CH₃)CF₃ | CH₃ |
| A-48 | (±)CH₂CH(CF₃)CH₂CH₃ | CH₃ |
| A-49 | (R)CH₂CH(CF₃)CH₂CH₃ | CH₃ |
| A-50 | (S)CH₂CH(CF₃)CH₂CH₃ | CH₃ |
| A-51 | (±,±)CH(CH₃)CH(CH₃)CF₃ | CH₃ |
| A-52 | (±,R)CH(CH₃)CH(CH₃)CF₃ | CH₃ |
| A-53 | (±,S)CH(CH₃)CH(CH₃)CF₃ | CH₃ |
| A-54 | (±,±)CH(CH₃)CH(CF₃)CH₂CH₃ | CH₃ |
| A-55 | (±,R)CH(CH₃)CH(CF₃)CH₂CH₃ | CH₃ |
| A-56 | (±,S)CH(CH₃)CH(CF₃)CH₂CH₃ | CH₃ |
| A-57 | CF₃ | CH₃ |
| A-58 | CF₂CF₃ | CH₃ |
| A-59 | CF₂CF₂CF₃ | CH₃ |
| A-60 | cyclo-C₃H₅ | CH₃ |
| A-61 | (1-CH₃)-cyclo-C₃H₄ | CH₃ |
| A-62 | cyclo-C₅H₉ | CH₃ |
| A-63 | cyclo-C₆H₁₁ | CH₃ |
| A-64 | (4-CH₃)-cyclo-C₆H₁₀ | CH₃ |
| A-65 | CH₂C(CH₃)═CH₂ | CH₃ |
| A-66 | CH₂CH₂C(CH₃)═CH₂ | CH₃ |
| A-67 | CH₂—C(CH₃)₃ | CH₃ |
| A-68 | CH₂—Si(CH₃)₃ | CH₃ |
| A-69 | n-C₆H₁₃ | CH₃ |
| A-70 | (CH₂)₃—CH(CH₃)₂ | CH₃ |
| A-71 | (CH₂)₂—CH(CH₃)—C₂H₅ | CH₃ |
| A-72 | CH₂—CH(CH₃)-n-C₃H₇ | CH₃ |
| A-73 | CH(CH₃)-n-C₄H₉ | CH₃ |
| A-74 | CH₂—CH(C₂H₅)₂ | CH₃ |
| A-75 | CH(C₂H₅)-n-C₃H₇ | CH₃ |
| A-76 | CH₂-cyclo-C₅H₉ | CH₃ |
| A-77 | CH₂—CH(CH₃)—CH(CH₃)₂ | CH₃ |
| A-78 | CH(CH₃)—CH₂CH(CH₃)₂ | CH₃ |
| A-79 | CH(CH₃)—CH(CH₃)—C₂H₅ | CH₃ |
| A-80 | CH(CH₃)—C(CH₃)₃ | CH₃ |
| A-81 | (CH₂)₂—C(CH₃)₃ | CH₃ |
| A-82 | CH₂—C(CH₃)₂—C₂H₅ | CH₃ |
| A-83 | 2-CH₃-cyclo-C₅H₈ | CH₃ |
| A-84 | 3-CH₃-cyclo-C₅H₈ | CH₃ |
| A-85 | C(CH₃)₂-n-C₃H₇ | CH₃ |
| A-86 | (CH₂)₆—CH₃ | CH₃ |
| A-87 | (CH₂)₄—CH(CH₃)₂ | CH₃ |
| A-88 | (CH₂)₃—CH(CH₃)—C₂H₅ | CH₃ |
| A-89 | (CH₂)₂—CH(CH₃)-n-C₃H₇ | CH₃ |
| A-90 | CH₂—CH(CH₃)-n-C₄H₉ | CH₃ |
| A-91 | CH(CH₃)-n-C₅H₁₁ | CH₃ |
| A-92 | (CH₂)₃C(CH₃)₃ | CH₃ |
| A-93 | (CH₂)₂CH(CH₃)—CH(CH₃)₂ | CH₃ |
| A-94 | (CH₂)CH(CH₃)—CH₂CH(CH₃)₂ | CH₃ |
| A-95 | CH(CH₃)(CH₂)₂—CH(CH₃)₂ | CH₃ |
| A-96 | (CH₂)₂C(CH₃)₂C₂H₅ | CH₃ |
| A-97 | CH₂CH(CH₃)CH(CH₃)C₂H₅ | CH₃ |
| A-98 | CH(CH₃)CH₂CH(CH₃)C₂H₅ | CH₃ |
| A-99 | CH₂C(CH₃)₂-n-C₃H₇ | CH₃ |
| A-100 | CH(CH₃)CH(CH₃)-n-C₃H₇ | CH₃ |
| A-101 | C(CH₃)₂-n-C₄H₉ | CH₃ |
| A-102 | (CH₂)₂CH(C₂H₅)₂ | CH₃ |
| A-103 | CH₂CH(C₂H₅)-n-C₃H₇ | CH₃ |
| A-104 | CH(C₂H₅)-n-C₄H₉ | CH₃ |
| A-105 | CH₂CH(CH₃)C(CH₃)₃ | CH₃ |
| A-106 | CH(CH₃)CH₂C(CH₃)₃ | CH₃ |
| A-107 | CH₂C(CH₃)₂CH(CH₃)₂ | CH₃ |
| A-108 | CH₂CH(C₂H₅)CH(CH₃)₂ | CH₃ |
| A-109 | CH(CH₃)CH(CH₃)CH(CH₃)₂ | CH₃ |
| A-110 | C(CH₃)₂CH₂CH(CH₃)₂ | CH₃ |
| A-111 | CH(C₂H₅)CH₂CH(CH₃)₂ | CH₃ |
| A-112 | CH(CH₃)C(CH₃)₂C₂H₅ | CH₃ |
| A-113 | CH(CH₃)CH(C₂H₅)₂ | CH₃ |
| A-114 | C(CH₃)₂CH(CH₃)C₂H₅ | CH₃ |
| A-115 | CH(C₂H₅)CH(CH₃)C₂H₅ | CH₃ |
| A-116 | C(CH₃)(C₂H₅)-n-C₃H₇ | CH₃ |
| A-117 | CH(n-C₃H₇)₂ | CH₃ |
| A-118 | CH(n-C₃H₇)CH(CH₃)₂ | CH₃ |
| A-119 | C(CH₃)₂C(CH₃)₃ | CH₃ |
| A-120 | C(CH₃)(C₂H₅)—CH(CH₃)₂ | CH₃ |
| A-121 | C(C₂H₅)₃ | CH₃ |
| A-122 | (3-CH₃)-cyclo-C₆H₁₀ | CH₃ |
| A-123 | (2-CH₃)-cyclo-C₆H₁₀ | CH₃ |
| A-124 | n-C₈H₁₇ | CH₃ |
| A-125 | CH₂C(═NO—CH₃)CH₃ | CH₃ |
| A-126 | CH₂C(═NO—C₂H₅)CH₃ | CH₃ |
| A-127 | CH₂C(═NO-n-C₃H₇)CH₃ | CH₃ |
| A-128 | CH₂C(═NO-i-C₃H₇)CH₃ | CH₃ |
| A-129 | CH(CH₃)C(═NOCH₃)CH₃ | CH₃ |
| A-130 | CH(CH₃)C(═NOC₂H₅)CH₃ | CH₃ |
| A-131 | CH(CH₃)C(═NO-n-C₃H₇)CH₃ | CH₃ |
| A-132 | CH(CH₃)C(═NO-i-C₃H₇)CH₃ | CH₃ |
| A-133 | CH₂C(═NO—CH₃)C₂H₅ | CH₃ |
| A-134 | CH₂C(═NO—C₂H₅)C₂H₅ | CH₃ |
| A-135 | CH₂C(50 NO-n-C₃H₇)C₂H₅ | CH₃ |
| A-136 | CH₂C(═NO-i-C₃H₇)C₂H₅ | CH₃ |
| A-137 | CH(CH₃)C(═NOCH₃)C₂H₅ | CH₃ |
| A-138 | CH(CH₃)C(═NOC₂H₅)C₂H₅ | CH₃ |
| A-139 | CH(CH₃)C(═NO-n-C₃H₇)C₂H₅ | CH₃ |
| A-140 | CH(CH₃)C(═NO-n-C₃H₇)C₂H₅ | CH₃ |
| A-141 | CH═CH—CH₂CH₃ | CH₃ |
| A-142 | CH₂—CH═CH—CH₃ | CH₃ |
| A-143 | CH₂—CH₂—CH═CH₂ | CH₃ |
| A-144 | C(CH₃)₂CH₂CH₃ | CH₃ |
| A-145 | CH═C(CH₃)₂ | CH₃ |
| A-146 | C(═CH₂)—CH₂CH₃ | CH₃ |
| A-147 | C(CH₃)═CH—CH₃ | CH₃ |
| A-148 | CH(CH₃)CH═CH₂ | CH₃ |
| A-149 | CH═CH-n-C₃H₇ | CH₃ |
| A-150 | CH₂—CH═CH—C₂H₅ | CH₃ |
| A-151 | (CH₂)₂—CH═CH—CH₃ | CH₃ |
| A-152 | (CH₂)₃—CH═CH₂ | CH₃ |
| A-153 | CH═CH—CH(CH₃)₂ | CH₃ |
| A-154 | CH₂—CH═C(CH₃)₂ | CH₃ |
| A-155 | (CH₂)₂—C(CH₃)═CH₂ | CH₃ |
| A-156 | CH═C(CH₃)—C₂H₅ | CH₃ |
| A-157 | CH₂—C(═CH₂)—C₂H₅ | CH₃ |
| A-158 | CH₂—C(CH₃)═CH—CH₃ | CH₃ |
| A-159 | CH₂—CH(CH₃)—CH═CH₂ | CH₃ |
| A-160 | C(═CH₂)—CH₂—CH₂—CH₃ | CH₃ |
| A-161 | C(CH₃)═CH—CH₂—CH₃ | CH₃ |
| A-162 | CH(CH₃)—CH═CH—CH₃ | CH₃ |
| A-163 | CH(CH₃)—CH₂—CH═CH₂ | CH₃ |
| A-164 | C(═CH₂)CH(CH₃)₂ | CH₃ |
| A-165 | C(CH₃)═C(CH₃)₂ | CH₃ |
| A-166 | CH(CH₃)—C(═CH₂)—CH₃ | CH₃ |
| A-167 | C(CH₃)₂—CH═CH₂ | CH₃ |
| A-168 | C(C₂H₅)═CH—CH₃ | CH₃ |
| A-169 | CH(C₂H₅)—CH═CH₂ | CH₃ |
| A-170 | CH═CH—CH₂—CH₂—CH₂—CH₃ | CH₃ |
| A-171 | CH₂—CH═CH—CH₂—CH₂—CH₃ | CH₃ |
| A-172 | CH₂—CH₂—CH═CH—CH₂—CH₃ | CH₃ |
| A-173 | CH₂—CH₂—CH₂—CH═CH—CH₃ | CH₃ |
| A-174 | CH₂—CH₂—CH₂—CH₂—CH═CH₂ | CH₃ |
| A-175 | CH═CH—CH₂—CH(CH₃)CH₃ | CH₃ |
| A-176 | CH₂—CH═CH—CH(CH₃)CH₃ | CH₃ |
| A-177 | CH₂—CH₂—CH═C(CH₃)CH₃ | CH₃ |
| A-178 | CH₂—CH₂—CH₂—C(CH₃)═CH₂ | CH₃ |
| A-179 | CH═CH—CH(CH₃)—CH₂—CH₃ | CH₃ |
| A-180 | CH₂—CH═C(CH₃)—CH₂—CH₃ | CH₃ |
| A-181 | CH₂—CH₂—C(═CH₂)—CH₂—CH₃ | CH₃ |
| A-182 | CH₂—CH₂—C(CH₃)═CH—CH₃ | CH₃ |
| A-183 | CH₂—CH₂—CH(CH₃)—CH═CH₂ | CH₃ |
| A-184 | CH═C(CH₃)—CH₂—CH₂—CH₃ | CH₃ |
| A-185 | CH₂—C(═CH₂)—CH₂—CH₂—CH₃ | CH₃ |
| A-186 | CH₂—C(CH₃)═CH—CH₂—CH₃ | CH₃ |
| A-187 | CH₂—CH(CH₃)—CH═CH—CH₃ | CH₃ |
| A-188 | CH₂—CH(CH₃)—CH₂—CH═CH₂ | CH₃ |
| A-189 | C(═CH₂)—CH₂—CH₂—CH₂—CH₃ | CH₃ |
| A-190 | C(CH₃)═CH—CH₂—CH₂—CH₃ | CH₃ |
| A-191 | CH(CH₃)—CH═CH—CH₂—CH₃ | CH₃ |
| A-192 | CH(CH₃)—CH₂—CH═CH—CH₃ | CH₃ |
| A-193 | CH(CH₃)—CH₂—CH₂—CH═CH₂ | CH₃ |
| A-194 | CH═CH—C(CH₃)₃ | CH₃ |
| A-195 | CH═C(CH₃)—CH(CH₃)—CH₃ | CH₃ |
| A-196 | CH₂—C(═CH₂)—CH(CH₃)—CH₃ | CH₃ |
| A-197 | CH₂—C(CH₃)═C(CH₃)—CH₃ | CH₃ |
| A-198 | CH₂—CH(CH₃)—C(═CH₂)—CH₃ | CH₃ |
| A-199 | C(═CH₂)—CH₂—CH(CH₃)—CH₃ | CH₃ |
| A-200 | C(CH₃)═CH—CH(CH₃)—CH₃ | CH₃ |
| A-201 | CH(CH₃)—CH═(CH₃)—CH₃ | CH₃ |
| A-202 | CH(CH₃)—CH₂—C(═CH₂)—CH₃ | CH₃ |
| A-203 | CH═C(CH₂—CH₃)—CH₂—CH₃ | CH₃ |
| A-204 | CH₂—C(═CH—CH₃)—CH₂—CH₃ | CH₃ |
| A-205 | CH₂—CH(CH═CH₂)—CH₂—CH₃ | CH₃ |
| A-206 | C(═CH—CH₃)—CH₂—CH₂—CH₃ | CH₃ |
| A-207 | CH(CH═CH₂)—CH₂—CH₂—CH₃ | CH₃ |
| A-208 | C(CH₂—CH₃)═CH—CH₂—CH₃ | CH₃ |
| A-209 | CH(CH₂—CH₃)—CH═CH—CH₃ | CH₃ |
| A-210 | CH(CH₂—CH₃)—CH₂—CH═CH₂ | CH₃ |
| A-211 | CH₂—C(CH₃)₂—CH═CH₂ | CH₃ |
| A-212 | C(═CH₂)—CH(CH₃)—CH₂—CH₃ | CH₃ |
| A-213 | C(CH₃)═C(CH₃)—CH₂—CH₃ | CH₃ |
| A-214 | CH(CH₃)—C(═CH₂)—CH₂—CH₃ | CH₃ |
| A-215 | CH(CH₃)—C(CH₃)═CH—CH₃ | CH₃ |
| A-216 | CH(CH₃)—CH(CH₃)—CH═CH₂ | CH₃ |
| A-217 | C(CH₃)₂—CH═CH—CH₃ | CH₃ |
| A-218 | C(CH₃)₂—CH₂—CH═CH₂ | CH₃ |
| A-219 | C(═CH₂)—C(CH₃)₃ | CH₃ |
| A-220 | C(═CH—CH₃)—CH(CH₃)—CH₃ | CH₃ |
| A-221 | CH(CH═CH₂)—CH(CH₃)—CH₃ | CH₃ |
| A-222 | C(CH₂—CH₃)═C(CH₃)—CH₃ | CH₃ |
| A-223 | CH(CH₂—CH₃)—C(═CH₂)—CH₃ | CH₃ |
| A-224 | C(CH₃)₂—C(═CH₂)—CH₃ | CH₃ |
| A-225 | C(CH₃)(CH═CH₂)—CH₂—CH₃ | CH₃ |
| A-226 | C(CH₃)(CH₂CH₃)—CH₂—CH₂—CH₃ | CH₃ |
| A-227 | CH(CH₂CH₃)—CH(CH₃)—CH₂—CH₃ | CH₃ |
| A-228 | CH(CH₂CH₃)—CH₂—CH(CH₃)—CH₃ | CH₃ |
| A-229 | C(CH₃)₂—C(CH₃)₃ | CH₃ |
| A-230 | C(CH₂—CH₃)—C(CH₃)₃ | CH₃ |
| A-231 | C(CH₃)(CH₂—CH₃)—CH(CH₃)₂ | CH₃ |
| A-232 | CH(CH(CH₃)₂)—CH(CH₃)₂ | CH₃ |
| A-233 | CH═CH—CH₂—CH₂—CH₂—CH₂—CH₃ | CH₃ |
| A-234 | CH₂—CH═CH—CH₂—CH₂—CH₂—CH₃ | CH₃ |
| A-235 | CH₂—CH₂—CH═CH—CH₂—CH₂—CH₃ | CH₃ |
| A-236 | CH₂—CH₂—CH₂—CH═CH—CH₂—CH₃ | CH₃ |
| A-237 | CH₂—CH₂—CH₂—CH₂—CH═CH—CH₃ | CH₃ |
| A-238 | CH₂—CH₂—CH₂—CH₂—CH₂—CH═CH₂ | CH₃ |
| A-239 | CH═CH—CH₂—CH₂—CH(CH₃)—CH₃ | CH₃ |
| A-240 | CH₂—CH═CH—CH₂—CH(CH₃)—CH₃ | CH₃ |
| A-241 | CH₂—CH₂—CH═CH—CH(CH₃)—CH₃ | CH₃ |
| A-242 | CH₂—CH₂—CH₂—CH═C(CH₃)—CH₃ | CH₃ |
| A-243 | CH₂—CH₂—CH₂—CH₂—C(═CH₂)—CH₃ | CH₃ |
| A-244 | CH═CH—CH₂—CH(CH₃)—CH₂—CH₃ | CH₃ |
| A-245 | CH₂—CH═CH—CH(CH₃)—CH₂—CH₃ | CH₃ |
| A-246 | CH₂—CH₂—CH═C(CH₃)—CH₂—CH₃ | CH₃ |
| A-247 | CH₂—CH₂—CH₂—C(═CH₂)—CH₂—CH₃ | CH₃ |
| A-248 | CH₂—CH₂—CH₂—C(CH₃)═CH—CH₃ | CH₃ |
| A-249 | CH₂—CH₂—CH₂—CH(CH₃)—CH═CH₂ | CH₃ |
| A-250 | CH═CH—CH(CH₃)—CH₂—CH₂—CH₃ | CH₃ |
| A-251 | CH₂—CH═C(CH₃)—CH₂—CH₂—CH₃ | CH₃ |
| A-252 | CH₂—CH₂—C(═CH₂)—CH₂—CH₂—CH₃ | CH₃ |
| A-253 | CH₂—CH₂—C(CH₃)═CH—CH₂—CH₃ | CH₃ |
| A-254 | CH₂—CH₂—CH(CH₃)—CH═CH—CH₃ | CH₃ |
| A-255 | CH₂—CH₂—CH(CH₃)—CH₂—CH═CH₂ | CH₃ |
| A-256 | CH═C(CH₃)—CH₂—CH₂—CH₂—CH₃ | CH₃ |
| A-257 | CH₂—C(═CH₂)—CH₂—CH₂—CH₂—CH₃ | CH₃ |
| A-258 | CH₂—C(CH₃)═CH—CH₂—CH₂—CH₃ | CH₃ |
| A-259 | CH₂—CH(CH₃)—CH═CH—CH₂—CH₃ | CH₃ |
| A-260 | CH₂—CH(CH₃)—CH₂—CH═CH—CH₃ | CH₃ |
| A-261 | CH₂—CH(CH₃)—CH₂—CH₂—CH═CH₂ | CH₃ |
| A-262 | C(═CH₂)—CH₂—CH₂—CH₂—CH₂—CH₃ | CH₃ |
| A-263 | C(CH₃)═CH—CH₂—CH₂—CH₂—CH₃ | CH₃ |
| A-264 | CH(CH₃)—CH═CH—CH₂—CH₂—CH₃ | CH₃ |
| A-265 | CH(CH₃)—CH₂—CH═CH—CH₂—CH₃ | CH₃ |
| A-266 | CH(CH₃)—CH₂—CH₂—CH═CH—CH₃ | CH₃ |
| A-267 | CH(CH₃)—CH₂—CH₂—CH₂—CH═CH₂ | CH₃ |
| A-268 | CH═CH—CH₂—C(CH₃)₃ | CH₃ |
| A-269 | CH₂—CH═CH—C(CH₃)₃ | CH₃ |
| A-270 | CH═CH—CH(CH₃)—CH(CH₃)₂ | CH₃ |
| A-271 | CH₂—CH═C(CH₃)—CH(CH₃)₂ | CH₃ |
| A-272 | CH₂—CH₂—C(═CH₂)—CH(CH₃)₂ | CH₃ |
| A-273 | CH₂—CH₂—C(CH₃)═C(CH₃)₂ | CH₃ |
| A-274 | CH₂—CH₂—CH(CH₃)—C(═CH₂)—CH₃ | CH₃ |
| A-275 | CH═C(CH₃)—CH₂—CH(CH₃)₂ | CH₃ |
| A-276 | CH₂—C(═CH₂)—CH₂—CH(CH₃)₂ | CH₃ |
| A-277 | CH₂—C(CH₃)═CH—CH(CH₃)₂ | CH₃ |
| A-278 | CH₂—CH(CH₃)—CH═C(CH₃)₂ | CH₃ |
| A-279 | CH₂—CH(CH₃)—CH₂—C(═CH₂)—CH₃ | CH₃ |
| A-280 | C(═CH₂)—CH₂—CH₂—CH(CH₃)₂ | CH₃ |
| A-281 | C(CH₃)═CH—CH₂—CH(CH₃)₂ | CH₃ |
| A-282 | CH(CH₃)—CH═CH—CH(CH₃)₂ | CH₃ |
| A-283 | CH(CH₃)—CH₂—CH═(CH₃)₂ | CH₃ |
| A-284 | CH(CH₃)—CH₂—CH₂—C(═CH₂)—CH₃ | CH₃ |
| A-285 | CH═CH—C(CH₃)₂—CH₂—CH₃ | CH₃ |
| A-286 | CH₂—CH₂—C(CH₃)₂—CH═CH₂ | CH₃ |
| A-287 | CH═C(CH₃)—CH(CH₃)—CH₂—CH₃ | CH₃ |
| A-288 | CH₂—C(═CH₂)—CH(CH₃)—CH₂—CH₃ | CH₃ |
| A-289 | CH₂—C(CH₃)═C(CH₃)—CH₂—CH₃ | CH₃ |
| A-290 | CH₂—CH(CH₃)—C(═CH₂)—CH₂—CH₃ | CH₃ |
| A-291 | CH₂—CH(CH₃)—C(CH₃)═CH—CH₃ | CH₃ |
| A-292 | CH₂—CH(CH₃)—CH(CH₃)—CH═CH₂ | CH₃ |
| A-293 | C(═CH₂)—CH₂—CH(CH₃)—CH₂—CH₃ | CH₃ |
| A-294 | C(CH₃)═CH—CH(CH₃)—CH₂—CH₃ | CH₃ |
| A-295 | CH(CH₃)—CH═(CH₃)—CH₂—CH₃ | CH₃ |
| A-296 | CH(CH₃)—CH₂—C(═CH₂)—CH₂—CH₃ | CH₃ |
| A-297 | CH(CH₃)—CH₂—C(CH₃)═CH—CH₃ | CH₃ |
| A-298 | CH(CH₃)—CH₂—CH(CH₃)—CH═CH₂ | CH₃ |
| A-299 | CH₂—C(CH₃)₂—CH═CH—CH₃ | CH₃ |
| A-300 | CH₂—C(CH₃)₂—CH₂—CH═CH₂ | CH₃ |
| A-301 | C(═CH₂)—CH(CH₃)—CH₂—CH₂—CH₃ | CH₃ |
| A-302 | C(CH₃)═C(CH₃)—CH₂—CH₂—CH₃ | CH₃ |
| A-303 | CH(CH₃)—C(═CH₂)—CH₂—CH₂—CH₃ | CH₃ |
| A-304 | CH(CH₃)—C(CH₃)═CH—CH₂—CH₃ | CH₃ |
| A-305 | CH(CH₃)—CH(CH₃)—CH═CH—CH₃ | CH₃ |
| A-306 | CH(CH₃)—CH(CH₃)—CH₂—CH═CH₂ | CH₃ |
| A-307 | C(CH₃)₂—CH═CH—CH₂—CH₃ | CH₃ |
| A-308 | C(CH₃)₂—CH₂—CH═CH—CH₃ | CH₃ |
| A-309 | C(CH₃)₂—CH₂—CH₂—CH═CH₂ | CH₃ |
| A-310 | CH═CH—CH(CH₂—CH₃)—CH₂—CH₃ | CH₃ |
| A-311 | CH₂—CH═C(CH₂—CH₃)—CH₂—CH₃ | CH₃ |
| A-312 | CH₂—CH₂—C(═CH—CH₃)—CH₂—CH₃ | CH₃ |
| A-313 | CH₂—CH₂—CH(CH═CH₂)—CH₂—CH₃ | CH₃ |
| A-314 | CH═C(CH₂—CH₃)—CH₂—CH₂—CH₃ | CH₃ |
| A-315 | CH₂—C(═CH—CH₃)—CH₂—CH₂—CH₃ | CH₃ |
| A-316 | CH₂—CH(CH═CH₂)—CH₂—CH₂—CH₃ | CH₃ |
| A-317 | CH₂—C(CH₂—CH₃)═CH—CH₂—CH₃ | CH₃ |
| A-318 | CH₂—CH(CH₂—CH₃)—CH═CH—CH₃ | CH₃ |
| A-319 | CH₂—CH(CH₂—CH₃)—CH—CH═CH₂ | CH₃ |
| A-320 | C(═CH—CH₃)—CH₂—CH₂—CH₂—CH₃ | CH₃ |
| A-321 | CH(CH═CH₂)—CH₂—CH₂—CH₂—CH₃ | CH₃ |
| A-322 | C(CH₂—CH₃)═CH—CH₂—CH₂—CH₃ | CH₃ |
| A-323 | CH(CH₂—CH₃)—CH═CH—CH₂—CH₃ | CH₃ |
| A-324 | CH(CH₂—CH₃)—CH₂—CH═CH—CH₃ | CH₃ |
| A-325 | CH(CH₂—CH₃)—CH₂—CH₂—CH═CH₂ | CH₃ |
| A-326 | C(═CH—CH₂—CH₃)—CH₂—CH₂—CH₃ | CH₃ |
| A-327 | C(CH═CH—CH₃)—CH₂—CH₂—CH₃ | CH₃ |
| A-328 | C(CH₂—CH═CH₂)—CH₂—CH₂—CH₃ | CH₃ |
| A-329 | CH═C(CH₃)—C(CH₃)₃ | CH₃ |
| A-330 | CH₂—C(═CH₂)—C(CH₃)₃ | CH₃ |
| A-331 | CH₂—C(CH₃)₂—CH(═CH₂)—CH₃ | CH₃ |
| A-332 | C(═CH₂)—CH(CH₃)—CH(CH₃)—CH₃ | CH₃ |
| A-333 | C(CH₃)═C(CH₃)—CH(CH₃)—CH₃ | CH₃ |
| A-334 | CH(CH₃)—C(═CH₂)—CH(CH₃)—CH₃ | CH₃ |
| A-335 | CH(CH₃)—C(CH₃)═C(CH₃)—CH₃ | CH₃ |
| A-336 | CH(CH₃)—CH(CH₃)—C(═CH₂)—CH₃ | CH₃ |
| A-337 | C(CH₃)₂—CH═C(CH₃)—CH₃ | CH₃ |
| A-338 | C(CH₃)₂—CH₂—C(═CH₂)—CH₃ | CH₃ |
| A-339 | C(CH₃)₂—C(═CH₂)—CH₂—CH₃ | CH₃ |
| A-340 | C(CH₃)₂—C(CH₃)═CH—CH₃ | CH₃ |
| A-341 | C(CH₃)₂—CH(CH₃)CH═CH₂ | CH₃ |
| A-342 | CH(CH₂—CH₃)—CH₂—CH(CH₃)—CH₃ | CH₃ |
| A-343 | CH(CH₂—CH₃)—CH(CH₃)—CH₂—CH₃ | CH₃ |
| A-344 | C(CH₃)(CH₂—CH₃)—CH₂—CH₂—CH₃ | CH₃ |
| A-345 | CH(i-C₃H₇)—CH₂—CH₂—CH₃ | CH₃ |
| A-346 | CH═C(CH₂—CH₃)—CH(CH₃)—CH₃ | CH₃ |
| A-347 | CH₂—C(═CH—CH₃)—CH(CH₃)—CH₃ | CH₃ |
| A-348 | CH₂—CH(CH═CH₂)—CH(CH₃)—CH₃ | CH₃ |
| A-349 | CH₂—C(CH₂—CH₃)═C(CH₃)—CH₃ | CH₃ |
| A-350 | CH₂—CH(CH₂—CH₃)—C(═CH₂)—CH₃ | CH₃ |
| A-351 | CH₂—C(CH₃)(CH═CH₂)—CH₂—CH₃ | CH₃ |
| A-352 | C(═CH₂)—CH(CH₂—CH₃)—CH₂—CH₃ | CH₃ |
| A-353 | C(CH₃)═C(CH₂—CH₃)—CH₂—CH₃ | CH₃ |
| A-354 | CH(CH₃)—C(═CH—CH₃)—CH₂—CH₃ | CH₃ |
| A-355 | CH(CH₃)—CH(CH═CH₂)—CH₂—CH₃ | CH₃ |
| A-356 | CH═C(CH₂—CH₃)—CH(CH₃)—CH₃ | CH₃ |
| A-357 | CH₂—C(═CH—CH₃)—CH(CH₃)—CH₃ | CH₃ |
| A-358 | CH₂—CH(CH═CH₂)—CH(CH₃)—CH₃ | CH₃ |
| A-359 | CH₂—C(CH₂—CH₃)═C(CH₃)—CH₃ | CH₃ |
| A-360 | CH₂—CH(CH₂—CH₃)—C(═CH₂)—CH₃ | CH₃ |
| A-361 | C(═CH—CH₃)—CH₂—CH(CH₃)—CH₃ | CH₃ |
| A-362 | CH(CH═CH₂)—CH₂—CH(CH₃)—CH₃ | CH₃ |
| A-363 | C(CH₂—CH₃)═CH—CH(CH₃)—CH₃ | CH₃ |
| A-364 | CH(CH₂—CH₃)CH═C(CH₃)—CH₃ | CH₃ |
| A-365 | CH(CH₂—CH₃)CH₂—C(═CH₂)—CH₃ | CH₃ |
| A-366 | C(═CH—CH₃)CH(CH₃)—CH₂—CH₃ | CH₃ |
| A-367 | CH(CH═CH₂)CH(CH₃)—CH₂—CH₃ | CH₃ |
| A-368 | C(CH₂—CH₃)═C(CH₃)—CH₂—CH₃ | CH₃ |
| A-369 | CH(CH₂—CH₃)—C(═CH₂)—CH₂—CH₃ | CH₃ |
| A-370 | CH(CH₂—CH₃)—C(CH₃)═CH—CH₃ | CH₃ |
| A-371 | CH(CH₂—CH₃)—CH(CH₃)—CH═CH₂ | CH₃ |
| A-372 | C(CH₃)(CH═CH₂)—CH₂—CH₂—CH₃ | CH₃ |
| A-373 | C(CH₃)(CH₂—CH₃)—CH═CH—CH₃ | CH₃ |
| A-374 | C(CH₃)(CH₂—CH₃)—CH₂—CH═CH₂ | CH₃ |
| A-375 | C[═C(CH₃)—CH₃]—CH₂—CH₂—CH₃ | CH₃ |
| A-376 | CH[C(═CH₂)—CH₃]—CH₂—CH₂—CH₃ | CH₃ |
| A-377 | C(i-C₃H₇)═CH—CH₂—CH₃ | CH₃ |
| A-378 | CH(i-C₃H₇)—CH═CH—CH₃ | CH₃ |
| A-379 | CH(i-C₃H₇)—CH₂—CH═CH₂ | CH₃ |
| A-380 | C(═CH—CH₃)—C(CH₃)₃ | CH₃ |
| A-381 | CH(CH═CH₂)—C(CH₃)₃ | CH₃ |
| A-382 | C(CH₃)(CH═CH₂)CH(CH₃)—CH₃ | CH₃ |
| A-383 | C(CH₃)(CH₂—CH₃)C(═CH₂)—CH₃ | CH₃ |
| A-384 | 2-CH₃-cyclohex-1-enyl | CH₃ |
| A-385 | [2-(═CH₂)]-cyclo-C₆H₉ | CH₃ |
| A-386 | 2-CH₃-cyclohex-2-enyl | CH₃ |
| A-387 | 2-CH₃-cyclohex-3-enyl | CH₃ |
| A-388 | 2-CH₃-cyclohex-4-enyl | CH₃ |
| A-389 | 2-CH₃-cyclohex-5-enyl | CH₃ |
| A-390 | 2-CH₃-cyclohex-6-enyl | CH₃ |
| A-391 | 3-CH₃-cyclohex-1-enyl | CH₃ |
| A-392 | 3-CH₃-cyclohex-2-enyl | CH₃ |
| A-393 | [3-(═CH₂)]-cyclo-C₆H₉ | CH₃ |
| A-394 | 3-CH₃-cyclohex-3-enyl | CH₃ |
| A-395 | 3-CH₃-cyclohex-4-enyl | CH₃ |
| A-396 | 3-CH₃-cyclohex-5-enyl | CH₃ |
| A-397 | 3-CH₃-cyclohex-6-enyl | CH₃ |
| A-398 | 4-CH₃-cyclohex-1-enyl | CH₃ |
| A-399 | 4-CH₃-cyclohex-2-enyl | CH₃ |
| A-400 | 4-CH₃-cyclohex-3-enyl | CH₃ |
| A-401 | [4-(═CH₂)]-cyclo-C₆H₉ | CH₃ |
| A-402 | CH₂CF₃ | CH₃ |
| A-403 | CH₂CCl₃ | CH₃ |
| A-404 | CH₂(cyclo-C₃H₅) | CH₃ |
| A-405 | CH(CH₃)₂ | CH₂CH₃ |
| A-406 | CH(CH₃)₂ | CH₂CH₂CH₃ |
| A-407 | CH(CH₃)₂ | CH₂CH₂F |
| A-408 | CH(CH₃)₂ | H |
| A-409 | CH(CH₃)₂ | CH(CH₃)₂ |
| A-410 | CH(CH₃)₂ | CH₂C(CH₃)₃ |
| A-411 | CH(CH₃)₂ | CH₂CH(CH₃)₂ |
| A-412 | CH(CH₃)₂ | (±)CH(CH₂CH₃)CH₃ |
| A-413 | CH(CH₃)₂ | (R)CH(CH₂CH₃)CH₃ |
| A-414 | CH(CH₃)₂ | (S)CH(CH₂CH₃)CH₃ |
| A-415 | CH(CH₃)₂ | (±)CH(CH₃)—CH(CH₃)₂ |
| A-416 | CH(CH₃)₂ | (R)CH(CH₃)—CH(CH₃)₂ |
| A-417 | CH(CH₃)₂ | (S)CH(CH₃)—CH(CH₃)₂ |
| A-418 | CH(CH₃)₂ | (±)CH(CH₃)—C(CH₃)₃ |
| A-419 | CH(CH₃)₂ | (R)CH(CH₃)—C(CH₃)₃ |
| A-420 | CH(CH₃)₂ | (S)CH(CH₃)—C(CH₃)₃ |
| A-421 | CH(CH₃)₂ | (±)CH(CH₃)—CF₃ |
| A-422 | CH(CH₃)₂ | (R)CH(CH₃)—CF₃ |
| A-423 | CH(CH₃)₂ | (S)CH(CH₃)—CF₃ |
| A-424 | CH(CH₃)₂ | (±)CH(CH₃)—CCl₃ |
| A-425 | CH(CH₃)₂ | (R)CH(CH₃)—CCl₃ |
| A-426 | CH(CH₃)₂ | (S)CH(CH₃)—CCl₃ |
| A-427 | CH(CH₃)₂ | CH₂C(CH₃)═CH₂ |
| A-428 | CH(CH₃)₂ | cyclopentyl |
| A-429 | CH(CH₃)₂ | cyclohexyl |
| A-430 | CH(CH₃)₂ | (CH₂)₃CH₃ |
| A-431 | CH(CH₃)₂ | C(CH₃)₃ |
| A-432 | CH(CH₃)₂ | (CH₂)₄CH₃ |
| A-433 | CH(CH₃)₂ | CH(CH₂CH₃)₂ |
| A-434 | CH(CH₃)₂ | CH₂CH₂CH(CH₃)₂ |
| A-435 | CH(CH₃)₂ | (±)CH(CH₃)(CH₂)₂CH₃ |
| A-436 | CH(CH₃)₂ | (R)CH(CH₃)(CH₂)₂CH₃ |
| A-437 | CH(CH₃)₂ | (S)CH(CH₃)(CH₂)₂CH₃ |
| A-438 | CH(CH₃)₂ | (±)CH₂CH(CH₃)CH₂CH₃ |
| A-439 | CH(CH₃)₂ | (R)CH₂CH(CH₃)CH₂CH₃ |
| A-440 | CH(CH₃)₂ | (S)CH₂CH(CH₃)CH₂CH₃ |
| A-441 | CH(CH₃)₂ | (±)CH(CH₃)CH(CH₃)₂ |
| A-442 | CH(CH₃)₂ | (R)CH(CH₃)CH(CH₃)₂ |
| A-443 | CH(CH₃)₂ | (S)CH(CH₃)CH(CH₃)₂ |
| A-444 | CH(CH₃)₂ | (CH₂)₅CH₃ |
| A-445 | CH(CH₃)₂ | (±,±)CH(CH₃)CH(CH₃)CH₂CH₃ |
| A-446 | CH(CH₃)₂ | (±,R)CH(CH₃)CH(CH₃)CH₂CH₃ |
| A-447 | CH(CH₃)₂ | (±,S)CH(CH₃)CH(CH₃)CH₂CH₃ |
| A-448 | CH(CH₃)₂ | (±)CH₂CH(CH₃)CF₃ |
| A-449 | CH(CH₃)₂ | (R)CH₂CH(CH₃)CF₃ |
| A-450 | CH(CH₃)₂ | (S)CH₂CH(CH₃)CF₃ |
| A-451 | CH(CH₃)₂ | (±)CH₂CH(CF₃)CH₂CH₃ |
| A-452 | CH(CH₃)₂ | (R)CH₂CH(CF₃)CH₂CH₃ |
| A-453 | CH(CH₃)₂ | (S)CH₂CH(CF₃)CH₂CH₃ |
| A-454 | CH(CH₃)₂ | (±,±)CH(CH₃)CH(CH₃)CF₃ |
| A-455 | CH(CH₃)₂ | (±,R)CH(CH₃)CH(CH₃)CF₃ |
| A-456 | CH(CH₃)₂ | (±,S)CH(CH₃)CH(CH₃)CF₃ |
| A-457 | CH(CH₃)₂ | (±,±)CH(CH₃)CH(CF₃)CH₂CH₃ |
| A-458 | CH(CH₃)₂ | (±,R)CH(CH₃)CH(CF₃)CH₂CH₃ |
| A-459 | CH(CH₃)₂ | (±,S)CH(CH₃)CH(CF₃)CH₂CH₃ |
| A-460 | CH(CH₃)₂ | CF₃ |
| A-461 | CH(CH₃)₂ | CF₂CF₃ |
| A-462 | CH(CH₃)₂ | CF₂CF₂CF₃ |
| A-463 | CH(CH₃)₂ | cyclo-C₃H₅ |
| A-464 | CH(CH₃)₂ | (1-CH₃)-cyclo-C₃H₄ |
| A-465 | CH(CH₃)₂ | cyclo-C₅H₉ |
| A-466 | CH(CH₃)₂ | cyclo-C₆H₁₁ |
| A-467 | CH(CH₃)₂ | (4-CH₃)-cyclo-C₆H₁₀ |
| A-468 | CH(CH₃)₂ | CH₂C(CH₃)═CH₂ |
| A-469 | CH(CH₃)₂ | CH₂CH₂C(CH₃)═CH₂ |
| A-470 | CH(CH₃)₂ | CH₂—C(CH₃)₃ |
| A-471 | CH(CH₃)₂ | CH₂—Si(CH₃)₃ |
| A-472 | CH(CH₃)₂ | n-C₆H₁₃ |
| A-473 | CH(CH₃)₂ | (CH₂)₃—CH(CH₃)₂ |
| A-474 | CH(CH₃)₂ | (CH₂)₂—CH(CH₃)—C₂H₅ |
| A-475 | CH(CH₃)₂ | CH₂—CH(CH₃)-n-C₃H₇ |
| A-476 | CH(CH₃)₂ | CH(CH₃)-n-C₄H₉ |
| A-477 | CH(CH₃)₂ | CH₂—CH(C₂H₅)₂ |
| A-478 | CH(CH₃)₂ | CH(C₂H₅)-n-C₃H₇ |
| A-479 | CH(CH₃)₂ | CH₂-cyclo-C₅H₉ |
| A-480 | CH(CH₃)₂ | CH₂—CH(CH₃)—CH(CH₃)₂ |
| A-481 | CH(CH₃)₂ | CH(CH₃)—CH₂CH(CH₃)₂ |
| A-482 | CH(CH₃)₂ | CH(CH₃)—CH(CH₃)—C₂H₅ |
| A-483 | CH(CH₃)₂ | CH(CH₃)—C(CH₃)₃ |
| A-484 | CH(CH₃)₂ | (CH₂)₂—C(CH₃)₃ |
| A-485 | CH(CH₃)₂ | CH₂—C(CH₃)₂—C₂H₅ |
| A-486 | CH(CH₃)₂ | 2-CH₃-cyclo-C₅H₈ |
| A-487 | CH(CH₃)₂ | 3-CH₃-cyclo-C₅H₈ |
| A-488 | CH(CH₃)₂ | C(CH₃)₂-n-C₃H₇ |
| A-489 | CH(CH₃)₂ | (CH₂)₆—CH₃ |
| A-490 | CH(CH₃)₂ | (CH₂)₄—CH(CH₃)₂ |
| A-491 | CH(CH₃)₂ | (CH₂)₃—CH(CH₃)—C₂H₅ |
| A-492 | CH(CH₃)₂ | (CH₂)₂—CH(CH₃)-n-C₃H₇ |
| A-493 | CH(CH₃)₂ | CH₂—CH(CH₃)-n-C₄H₉ |
| A-494 | CH(CH₃)₂ | CH(CH₃)-n-C₅H₁₁ |
| A-495 | CH(CH₃)₂ | (CH₂)₃C(CH₃)₃ |
| A-496 | CH(CH₃)₂ | (CH₂)₂CH(CH₃)—CH(CH₃)₂ |
| A-497 | CH(CH₃)₂ | (CH₂)CH(CH₃)—CH₂CH(CH₃)₂ |
| A-498 | CH(CH₃)₂ | CH(CH₃)(CH₂)₂—CH(CH₃)₂ |
| A-499 | CH(CH₃)₂ | (CH₂)₂C(CH₃)₂C₂H₅ |
| A-500 | CH(CH₃)₂ | CH₂CH(CH₃)CH(CH₃)C₂H₅ |
| A-501 | CH(CH₃)₂ | CH(CH₃)CH₂CH(CH₃)C₂H₅ |
| A-502 | CH(CH₃)₂ | CH₂C(CH₃)₂-n-C₃H₇ |
| A-503 | CH(CH₃)₂ | CH(CH₃)CH(CH₃)-n-C₃H₇ |
| A-504 | CH(CH₃)₂ | C(CH₃)₂-n-C₄H₉ |
| A-505 | CH(CH₃)₂ | (CH₂)₂CH(C₂H₅)₂ |
| A-506 | CH(CH₃)₂ | CH₂CH(C₂H₅)-n-C₃H₇ |
| A-507 | CH(CH₃)₂ | CH(C₂H₅)-n-C₄H₉ |
| A-508 | CH(CH₃)₂ | CH₂CH(CH₃)C(CH₃)₃ |
| A-509 | CH(CH₃)₂ | CH(CH₃)CH₂C(CH₃)₃ |
| A-510 | CH(CH₃)₂ | CH₂C(CH₃)₂CH(CH₃)₂ |
| A-511 | CH(CH₃)₂ | CH₂CH(C₂H₅)CH(CH₃)₂ |
| A-512 | CH(CH₃)₂ | CH(CH₃)CH(CH₃)CH(CH₃)₂ |
| A-513 | CH(CH₃)₂ | C(CH₃)₂CH₂CH(CH₃)₂ |
| A-514 | CH(CH₃)₂ | CH(C₂H₅)CH₂CH(CH₃)₂ |
| A-515 | CH(CH₃)₂ | CH(CH₃)C(CH₃)₂C₂H₅ |
| A-516 | CH(CH₃)₂ | CH(CH₃)CH(C₂H₅)₂ |
| A-517 | CH(CH₃)₂ | C(CH₃)₂CH(CH₃)C₂H₅ |
| A-518 | CH(CH₃)₂ | CH(C₂H₅)CH(CH₃)C₂H₅ |
| A-519 | CH(CH₃)₂ | C(CH₃)(C₂H₅)-n-C₃H₇ |
| A-520 | CH(CH₃)₂ | CH(n-C₃H₇)₂ |
| A-521 | CH(CH₃)₂ | CH(n-C₃H₇)CH(CH₃)₂ |
| A-522 | CH(CH₃)₂ | C(CH₃)₂C(CH₃)₃ |
| A-523 | CH(CH₃)₂ | C(CH₃)(C₂H₅)—CH(CH₃)₂ |
| A-524 | CH(CH₃)₂ | C(C₂H₅)₃ |
| A-525 | CH(CH₃)₂ | (3-CH₃)-cyclo-C₆H₁₀ |
| A-526 | CH(CH₃)₂ | (2-CH₃)-cyclo-C₆H₁₀ |
| A-527 | CH(CH₃)₂ | n-C₈H₁₇ |
| A-528 | CH(CH₃)₂ | CH₂C(═NO—CH₃)CH₃ |
| A-529 | CH(CH₃)₂ | CH₂C(═NO—C₂H₅)CH₃ |
| A-530 | CH(CH₃)₂ | CH₂C(═NO-n-C₃H₇)CH₃ |
| A-531 | CH(CH₃)₂ | CH₂C(═NO-i-C₃H₇)CH₃ |
| A-532 | CH(CH₃)₂ | CH(CH₃)C(═NOCH₃)CH₃ |
| A-533 | CH(CH₃)₂ | CH(CH₃)C(═NOC₂H₅)CH₃ |
| A-534 | CH(CH₃)₂ | CH(CH₃)C(═NO-n-C₃H₇)CH₃ |
| A-535 | CH(CH₃)₂ | CH(CH₃)C(═NO-i-C₃H₇)CH₃ |
| A-536 | CH(CH₃)₂ | CH₂C(═NO—CH₃)C₂H₅ |
| A-537 | CH(CH₃)₂ | CH₂C(═NO—C₂H₅)C₂H₅ |
| A-538 | CH(CH₃)₂ | CH₂C(═NO-n-C₃H₇)C₂H₅ |
| A-539 | CH(CH₃)₂ | CH₂C(═NO-i-C₃H₇)C₂H₅ |
| A-540 | CH(CH₃)₂ | CH(CH₃)C(═NOCH₃)C₂H₅ |
| A-541 | CH(CH₃)₂ | CH(CH₃)C(═NOC₂H₅)C₂H₅ |
| A-542 | CH(CH₃)₂ | CH(CH₃)C(═NO-n-C₃H₇)C₂H₅ |
| A-543 | CH(CH₃)₂ | CH(CH₃)C(═NO-n-C₃H₇)C₂H₅ |
| A-544 | CH(CH₃)₂ | CH═CH—CH₂CH₃ |
| A-545 | CH(CH₃)₂ | CH₂—CH═CH—CH₃ |
| A-546 | CH(CH₃)₂ | CH₂—CH₂—CH═CH₂ |
| A-547 | CH(CH₃)₂ | C(CH₃)₂CH₂CH₃ |
| A-548 | CH(CH₃)₂ | CH═C(CH₃)₂ |
| A-549 | CH(CH₃)₂ | C(═CH₂)—CH₂CH₃ |
| A-550 | CH(CH₃)₂ | C(CH₃)═CH—CH₃ |
| A-551 | CH(CH₃)₂ | CH(CH₃)CH═CH₂ |
| A-552 | CH(CH₃)₂ | CH═CH-n-C₃H₇ |
| A-553 | CH(CH₃)₂ | CH₂—CH═CH—C₂H₅ |
| A-554 | CH(CH₃)₂ | (CH₂)₂—CH═CH—CH₃ |
| A-555 | CH(CH₃)₂ | (CH₂)₃—CH═CH₂ |
| A-556 | CH(CH₃)₂ | CH═CH—CH(CH₃)₂ |
| A-557 | CH(CH₃)₂ | CH₂—CH═C(CH₃)₂ |
| A-558 | CH(CH₃)₂ | (CH₂)₂—C(CH₃)═CH₂ |
| A-559 | CH(CH₃)₂ | CH═C(CH₃)—C₂H₅ |
| A-560 | CH(CH₃)₂ | CH₂—C(═CH₂)—C₂H₅ |
| A-561 | CH(CH₃)₂ | CH₂—C(CH₃)═CH—CH₃ |
| A-562 | CH(CH₃)₂ | CH₂—CH(CH₃)—CH═CH₂ |
| A-563 | CH(CH₃)₂ | C(═CH₂)—CH₂—CH₂—CH₃ |
| A-564 | CH(CH₃)₂ | C(CH₃)═CH—CH₂—CH₃ |
| A-565 | CH(CH₃)₂ | CH(CH₃)—CH═CH—CH₃ |
| A-566 | CH(CH₃)₂ | CH(CH₃)—CH₂—CH═CH₂ |
| A-567 | CH(CH₃)₂ | C(═CH₂)CH(CH₃)₂ |
| A-568 | CH(CH₃)₂ | C(CH₃)═C(CH₃)₂ |
| A-569 | CH(CH₃)₂ | CH(CH₃)—C(═CH₂)—CH₃ |
| A-570 | CH(CH₃)₂ | C(CH₃)₂—CH═CH₂ |
| A-571 | CH(CH₃)₂ | C(C₂H₅)═CH—CH₃ |
| A-572 | CH(CH₃)₂ | CH(C₂H₅)—CH═CH₂ |
| A-573 | CH(CH₃)₂ | CH═CH—CH₂—CH₂—CH₂—CH₃ |
| A-574 | CH(CH₃)₂ | CH₂—CH═CH—CH₂—CH₂—CH₃ |
| A-575 | CH(CH₃)₂ | CH₂—CH₂—CH═CH—CH₂—CH₃ |
| A-576 | CH(CH₃)₂ | CH₂—CH₂—CH₂—CH═CH—CH₃ |
| A-577 | CH(CH₃)₂ | CH₂—CH₂—CH₂—CH₂—CH═CH₂ |
| A-578 | CH(CH₃)₂ | CH═CH—CH₂—CH(CH₃)CH₃ |
| A-579 | CH(CH₃)₂ | CH₂—CH═CH—CH(CH₃)CH₃ |
| A-580 | CH(CH₃)₂ | CH₂—CH₂—CH═C(CH₃)CH₃ |
| A-581 | CH(CH₃)₂ | CH₂—CH₂—CH₂—C(CH₃)═CH₂ |
| A-582 | CH(CH₃)₂ | CH═CH—CH(CH₃)—CH₂—CH₃ |
| A-583 | CH(CH₃)₂ | CH₂—CH═C(CH₃)—CH₂—CH₃ |
| A-584 | CH(CH₃)₂ | CH₂—CH₂—C(═CH₂)—CH₂—CH₃ |
| A-585 | CH(CH₃)₂ | CH₂—CH₂—C(CH₃)═CH—CH₃ |
| A-586 | CH(CH₃)₂ | CH₂—CH₂—CH(CH₃)—CH═CH₂ |
| A-587 | CH(CH₃)₂ | CH═C(CH₃)—CH₂—CH₂—CH₃ |
| A-588 | CH(CH₃)₂ | CH₂—C(═CH₂)—CH₂—CH₂—CH₃ |
| A-589 | CH(CH₃)₂ | CH₂—C(CH₃)═CH—CH₂—CH₃ |
| A-590 | CH(CH₃)₂ | CH₂—CH(CH₃)—CH═CH—CH₃ |
| A-591 | CH(CH₃)₂ | CH₂—CH(CH₃)—CH₂—CH═CH₂ |
| A-592 | CH(CH₃)₂ | C(═CH₂)—CH₂—CH₂—CH₂—CH₃ |
| A-593 | CH(CH₃)₂ | C(CH₃)═CH—CH₂—CH₂—CH₃ |
| A-594 | CH(CH₃)₂ | CH(CH₃)—CH═CH—CH₂—CH₃ |
| A-595 | CH(CH₃)₂ | CH(CH₃)—CH₂—CH═CH—CH₃ |
| A-596 | CH(CH₃)₂ | CH(CH₃)—CH₂—CH₂—CH═CH₂ |
| A-597 | CH(CH₃)₂ | CH═CH—C(CH₃)₃ |
| A-598 | CH(CH₃)₂ | CH═C(CH₃)—CH(CH₃)—CH₃ |
| A-599 | CH(CH₃)₂ | CH₂—C(═CH₂)—CH(CH₃)—CH₃ |
| A-600 | CH(CH₃)₂ | CH₂—C(CH₃)═C(CH₃)—CH₃ |
| A-601 | CH(CH₃)₂ | CH₂—CH(CH₃)—C(═CH₂)—CH₃ |
| A-602 | CH(CH₃)₂ | C(═CH₂)—CH₂—CH(CH₃)—CH₃ |
| A-603 | CH(CH₃)₂ | C(CH₃)═CH—CH(CH₃)—CH₃ |
| A-604 | CH(CH₃)₂ | CH(CH₃)—CH═C(CH₃)—CH₃ |
| A-605 | CH(CH₃)₂ | CH(CH₃)—CH₂—C(═CH₂)—CH₃ |
| A-606 | CH(CH₃)₂ | CH═C(CH₂—CH₃)—CH₂—CH₃ |
| A-607 | CH(CH₃)₂ | CH₂—C(═CH—CH₃)—CH₂—CH₃ |
| A-608 | CH(CH₃)₂ | CH₂—CH(CH═CH₂)—CH₂—CH₃ |
| A-609 | CH(CH₃)₂ | C(═CH—CH₃)—CH₂—CH₂—CH₃ |
| A-610 | CH(CH₃)₂ | CH(CH═CH₂)—CH₂—CH₂—CH₃ |
| A-611 | CH(CH₃)₂ | C(CH₂—CH₃)═CH—CH₂—CH₃ |
| A-612 | CH(CH₃)₂ | CH(CH₂—CH₃)—CH═CH—CH₃ |
| A-613 | CH(CH₃)₂ | CH(CH₂—CH₃)—CH₂—CH═CH₂ |
| A-614 | CH(CH₃)₂ | CH₂—C(CH₃)₂—CH═CH₂ |
| A-615 | CH(CH₃)₂ | C(═CH₂)—CH(CH₃)—CH₂—CH₃ |
| A-616 | CH(CH₃)₂ | C(CH₃)═C(CH₃)—CH₂—CH₃ |
| A-617 | CH(CH₃)₂ | CH(CH₃)—C(═CH₂)—CH₂—CH₃ |
| A-618 | CH(CH₃)₂ | CH(CH₃)—C(CH₃)═CH—CH₃ |
| A-619 | CH(CH₃)₂ | CH(CH₃)—CH(CH₃)—CH═CH₂ |
| A-620 | CH(CH₃)₂ | C(CH₃)₂—CH═CH—CH₃ |
| A-621 | CH(CH₃)₂ | C(CH₃)₂—CH₂—CH═CH₂ |
| A-622 | CH(CH₃)₂ | C(═CH₂)—C(CH₃)₃ |
| A-623 | CH(CH₃)₂ | C(═CH—CH₃)—CH(CH₃)—CH₃ |
| A-624 | CH(CH₃)₂ | CH(CH═CH₂)—CH(CH₃)—CH₃ |
| A-625 | CH(CH₃)₂ | C(CH₂—CH₃)═C(CH₃)—CH₃ |
| A-626 | CH(CH₃)₂ | CH(CH₂—CH₃)—C(═CH₂)—CH₃ |
| A-627 | CH(CH₃)₂ | C(CH₃)₂—C(═CH₂)—CH₃ |
| A-628 | CH(CH₃)₂ | C(CH₃)(CH═CH₂)—CH₂—CH₃ |
| A-629 | CH(CH₃)₂ | C(CH₃)(CH₂CH₃)—CH₂—CH₂—CH₃ |
| A-630 | CH(CH₃)₂ | CH(CH₂CH₃)—CH(CH₃)—CH₂—CH₃ |
| A-631 | CH(CH₃)₂ | CH(CH₂CH₃)—CH₂—CH(CH₃)—CH₃ |
| A-632 | CH(CH₃)₂ | C(CH₃)₂—C(CH₃)₃ |
| A-633 | CH(CH₃)₂ | C(CH₂—CH₃)—C(CH₃)₃ |
| A-634 | CH(CH₃)₂ | C(CH₃)(CH₂—CH₃)—CH(CH₃)₂ |
| A-635 | CH(CH₃)₂ | CH(CH(CH₃)₂)—CH(CH₃)₂ |
| A-636 | CH(CH₃)₂ | CH═CH—CH₂—CH₂—CH₂—CH₂—CH₃ |
| A-637 | CH(CH₃)₂ | CH₂—CH═CH—CH₂—CH₂—CH₂—CH₃ |
| A-638 | CH(CH₃)₂ | CH₂—CH₂—CH═CH—CH₂—CH₂—CH₃ |
| A-639 | CH(CH₃)₂ | CH₂—CH₂—CH₂—CH═CH—CH₂—CH₃ |
| A-640 | CH(CH₃)₂ | CH₂—CH₂—CH₂—CH₂—CH═CH—CH₃ |
| A-641 | CH(CH₃)₂ | CH₂—CH₂—CH₂—CH₂—CH₂—CH═CH₂ |
| A-642 | CH(CH₃)₂ | CH═CH—CH₂—CH₂—CH(CH₃)—CH₃ |
| A-643 | CH(CH₃)₂ | CH₂—CH═CH—CH₂—CH(CH₃)—CH₃ |
| A-644 | CH(CH₃)₂ | CH₂—CH₂—CH═CH—CH(CH₃)—CH₃ |
| A-645 | CH(CH₃)₂ | CH₂—CH₂—CH₂—CH═C(CH₃)—CH₃ |
| A-646 | CH(CH₃)₂ | CH₂—CH₂—CH₂—CH₂—C(═CH₂)—CH₃ |
| A-647 | CH(CH₃)₂ | CH═CH—CH₂—CH(CH₃)—CH₂—CH₃ |
| A-648 | CH(CH₃)₂ | CH₂—CH═CH—CH(CH₃)—CH₂—CH₃ |
| A-649 | CH(CH₃)₂ | CH₂—CH₂—CH═C(CH₃)—CH₂—CH₃ |
| A-650 | CH(CH₃)₂ | CH₂—CH₂—CH₂—C(═CH₂)—CH₂—CH₃ |
| A-651 | CH(CH₃)₂ | CH₂—CH₂—CH₂—C(CH₃)═CH—CH₃ |
| A-652 | CH(CH₃)₂ | CH₂—CH₂—CH₂—CH(CH₃)—CH═CH₂ |
| A-653 | CH(CH₃)₂ | CH═CH—CH(CH₃)—CH₂—CH₂—CH₃ |
| A-654 | CH(CH₃)₂ | CH₂—CH═C(CH₃)—CH₂—CH₂—CH₃ |
| A-655 | CH(CH₃)₂ | CH₂—CH₂—C(═CH₂)—CH₂—CH₂—CH₃ |
| A-656 | CH(CH₃)₂ | CH₂—CH₂—C(CH₃)═CH—CH₂—CH₃ |
| A-657 | CH(CH₃)₂ | CH₂—CH₂—CH(CH₃)—CH═CH—CH₃ |
| A-658 | CH(CH₃)₂ | CH₂—CH₂—CH(CH₃)—CH₂—CH═CH₂ |
| A-659 | CH(CH₃)₂ | CH═C(CH₃)—CH₂—CH₂—CH₂—CH₃ |
| A-660 | CH(CH₃)₂ | CH₂—C(═CH₂)—CH₂—CH₂—CH₂—CH₃ |
| A-661 | CH(CH₃)₂ | CH₂—C(CH₃)═CH—CH₂—CH₂—CH₃ |
| A-662 | CH(CH₃)₂ | CH₂—CH(CH₃)—CH═CH—CH₂—CH₃ |
| A-663 | CH(CH₃)₂ | CH₂—CH(CH₃)—CH₂—CH═CH—CH₃ |
| A-664 | CH(CH₃)₂ | CH₂—CH(CH₃)—CH₂—CH₂—CH═CH₂ |
| A-665 | CH(CH₃)₂ | C(═CH₂)—CH₂—CH₂—CH₂—CH₂—CH₃ |
| A-666 | CH(CH₃)₂ | C(CH₃)═CH—CH₂—CH₂—CH₂—CH₃ |
| A-667 | CH(CH₃)₂ | CH(CH₃)—CH═CH—CH₂—CH₂—CH₃ |
| A-668 | CH(CH₃)₂ | CH(CH₃)—CH₂—CH═CH—CH₂—CH₃ |
| A-669 | CH(CH₃)₂ | CH(CH₃)—CH₂—CH₂—CH═CH—CH₃ |
| A-670 | CH(CH₃)₂ | CH(CH₃)—CH₂—CH₂—CH₂—CH═CH₂ |
| A-671 | CH(CH₃)₂ | CH═CH—CH₂—C(CH₃)₃ |
| A-672 | CH(CH₃)₂ | CH₂—CH═CH—C(CH₃)₃ |
| A-673 | CH(CH₃)₂ | CH═CH—CH(CH₃)—CH(CH₃)₂ |
| A-674 | CH(CH₃)₂ | CH₂—CH═C(CH₃)—CH(CH₃)₂ |
| A-675 | CH(CH₃)₂ | CH₂—CH₂—C(═CH₂)—CH(CH₃)₂ |
| A-676 | CH(CH₃)₂ | CH₂—CH₂—C(CH₃)═C(CH₃)₂ |
| A-677 | CH(CH₃)₂ | CH₂—CH₂—CH(CH₃)—C(═CH₂)—CH₃ |
| A-678 | CH(CH₃)₂ | CH═C(CH₃)—CH₂—CH(CH₃)₂ |
| A-679 | CH(CH₃)₂ | CH₂—C(═CH₂)—CH₂—CH(CH₃)₂ |
| A-680 | CH(CH₃)₂ | CH₂—C(CH₃)═CH—CH(CH₃)₂ |
| A-681 | CH(CH₃)₂ | CH₂—CH(CH₃)—CH═C(CH₃)₂ |
| A-682 | CH(CH₃)₂ | CH₂—CH(CH₃)—CH₂—C(═CH₂)—CH₃ |
| A-683 | CH(CH₃)₂ | C(═CH₂)—CH₂—CH₂—CH(CH₃)₂ |
| A-684 | CH(CH₃)₂ | C(CH₃)═CH—CH₂—CH(CH₃)₂ |
| A-685 | CH(CH₃)₂ | CH(CH₃)—CH═CH—CH(CH₃)₂ |
| A-686 | CH(CH₃)₂ | CH(CH₃)—CH₂—CH═C(CH₃)₂ |
| A-687 | CH(CH₃)₂ | CH(CH₃)—CH₂—CH₂—C(═CH₂)—CH₃ |
| A-688 | CH(CH₃)₂ | CH═CH—C(CH₃)₂—CH₂—CH₃ |
| A-689 | CH(CH₃)₂ | CH₂—CH₂—C(CH₃)₂—CH═CH₂ |
| A-690 | CH(CH₃)₂ | CH═C(CH₃)—CH(CH₃)—CH₂—CH₃ |
| A-691 | CH(CH₃)₂ | CH₂—C(═CH₂)—CH(CH₃)—CH₂—CH₃ |
| A-692 | CH(CH₃)₂ | CH₂—C(CH₃)═C(CH₃)—CH₂—CH₃ |
| A-693 | CH(CH₃)₂ | CH₂—CH(CH₃)—C(═CH₂)—CH₂—CH₃ |
| A-694 | CH(CH₃)₂ | CH₂—CH(CH₃)—C(CH₃)═CH—CH₃ |
| A-695 | CH(CH₃)₂ | CH₂—CH(CH₃)—CH(CH₃)—CH═CH₂ |
| A-696 | CH(CH₃)₂ | C(═CH₂)—CH₂—CH(CH₃)—CH₂—CH₃ |
| A-697 | CH(CH₃)₂ | C(CH₃)═CH—CH(CH₃)—CH₂—CH₃ |
| A-698 | CH(CH₃)₂ | CH(CH₃)—CH═C(CH₃)—CH₂—CH₃ |
| A-699 | CH(CH₃)₂ | CH(CH₃)—CH₂—C(═CH₂)—CH₂—CH₃ |
| A-700 | CH(CH₃)₂ | CH(CH₃)—CH₂—C(CH₃)═CH—CH₃ |
| A-701 | CH(CH₃)₂ | CH(CH₃)—CH₂—CH(CH₃)—CH═CH₂ |
| A-702 | CH(CH₃)₂ | CH₂—C(CH₃)₂—CH═CH—CH₃ |
| A-703 | CH(CH₃)₂ | CH₂—C(CH₃)₂—CH₂—CH═CH₂ |
| A-704 | CH(CH₃)₂ | C(═CH₂)—CH(CH₃)—CH₂—CH₂—CH₃ |
| A-705 | CH(CH₃)₂ | C(CH₃)═C(CH₃)—CH₂—CH₂—CH₃ |
| A-706 | CH(CH₃)₂ | CH(CH₃)—C(═CH₂)—CH₂—CH₂—CH₃ |
| A-707 | CH(CH₃)₂ | CH(CH₃)—C(CH₃)═CH—CH₂—CH₃ |
| A-708 | CH(CH₃)₂ | CH(CH₃)—CH(CH₃)—CH═CH—CH₃ |
| A-709 | CH(CH₃)₂ | CH(CH₃)—CH(CH₃)—CH₂—CH═CH₂ |
| A-710 | CH(CH₃)₂ | C(CH₃)₂—CH═CH—CH₂—CH₃ |
| A-711 | CH(CH₃)₂ | C(CH₃)₂—CH₂—CH═CH—CH₃ |
| A-712 | CH(CH₃)₂ | C(CH₃)₂—CH₂—CH₂—CH═CH₂ |
| A-713 | CH(CH₃)₂ | CH═CH—CH(CH₂—CH₃)—CH₂—CH₃ |
| A-714 | CH(CH₃)₂ | CH₂—CH═C(CH₂—CH₃)—CH₂—CH₃ |
| A-715 | CH(CH₃)₂ | CH₂—CH₂—C(═CH—CH₃)—CH₂—CH₃ |
| A-716 | CH(CH₃)₂ | CH₂—CH₂—CH(CH═CH₂)—CH₂—CH₃ |
| A-717 | CH(CH₃)₂ | CH═C(CH₂—CH₃)—CH₂—CH₂—CH₃ |
| A-718 | CH(CH₃)₂ | CH₂—C(═CH—CH₃)—CH₂—CH₂—CH₃ |
| A-719 | CH(CH₃)₂ | CH₂—CH(CH═CH₂)—CH₂—CH₂—CH₃ |
| A-720 | CH(CH₃)₂ | CH₂—C(CH₂—CH₃)═CH—CH₂—CH₃ |
| A-721 | CH(CH₃)₂ | CH₂—CH(CH₂—CH₃)—CH═CH—CH₃ |
| A-722 | CH(CH₃)₂ | CH₂—CH(CH₂—CH₃)—CH—CH═CH₂ |
| A-723 | CH(CH₃)₂ | C(═CH—CH₃)—CH₂—CH₂—CH₂—CH₃ |
| A-724 | CH(CH₃)₂ | CH(CH═CH₂)—CH₂—CH₂—CH₂—CH₃ |
| A-725 | CH(CH₃)₂ | C(CH₂—CH₃)═CH—CH₂—CH₂—CH₃ |
| A-726 | CH(CH₃)₂ | CH(CH₂—CH₃)—CH═CH—CH₂—CH₃ |
| A-727 | CH(CH₃)₂ | CH(CH₂—CH₃)—CH₂—CH═CH—CH₃ |
| A-728 | CH(CH₃)₂ | CH(CH₂—CH₃)—CH₂—CH₂—CH═CH₂ |
| A-729 | CH(CH₃)₂ | C(═CH—CH₂—CH₃)—CH₂—CH₂—CH₃ |
| A-730 | CH(CH₃)₂ | C(CH═CH—CH₃)—CH₂—CH₂—CH₃ |
| A-731 | CH(CH₃)₂ | C(CH₂—CH═CH₂)—CH₂—CH₂—CH₃ |
| A-732 | CH(CH₃)₂ | CH═C(CH₃)—C(CH₃)₃ |
| A-733 | CH(CH₃)₂ | CH₂—C(═CH₂)—C(CH₃)₃ |
| A-734 | CH(CH₃)₂ | CH₂—C(CH₃)₂—CH(═CH₂)—CH₃ |
| A-735 | CH(CH₃)₂ | C(═CH₂)—CH(CH₃)—CH(CH₃)—CH₃ |
| A-736 | CH(CH₃)₂ | C(CH₃)═C(CH₃)—CH(CH₃)—CH₃ |
| A-737 | CH(CH₃)₂ | CH(CH₃)—C(═CH₂)—CH(CH₃)—CH₃ |
| A-738 | CH(CH₃)₂ | CH(CH₃)—C(CH₃)═C(CH₃)—CH₃ |
| A-739 | CH(CH₃)₂ | CH(CH₃)—CH(CH₃)—C(═CH₂)—CH₃ |
| A-740 | CH(CH₃)₂ | C(CH₃)₂—CH═C(CH₃)—CH₃ |
| A-741 | CH(CH₃)₂ | C(CH₃)₂—CH₂—C(═CH₂)—CH₃ |
| A-742 | CH(CH₃)₂ | C(CH₃)₂—C(═CH₂)—CH₂—CH₃ |
| A-743 | CH(CH₃)₂ | C(CH₃)₂—C(CH₃)═CH—CH₃ |
| A-744 | CH(CH₃)₂ | C(CH₃)₂—CH(CH₃)CH═CH₂ |
| A-745 | CH(CH₃)₂ | CH(CH₂—CH₃)—CH₂—CH(CH₃)—CH₃ |
| A-746 | CH(CH₃)₂ | CH(CH₂—CH₃)—CH(CH₃)—CH₂—CH₃ |
| A-747 | CH(CH₃)₂ | C(CH₃)(CH₂—CH₃)—CH₂—CH₂—CH₃ |
| A-748 | CH(CH₃)₂ | CH(i-C₃H₇)—CH₂—CH₂—CH₃ |
| A-749 | CH(CH₃)₂ | CH═C(CH₂—CH₃)—CH(CH₃)—CH₃ |
| A-750 | CH(CH₃)₂ | CH₂—C(═CH—CH₃)—CH(CH₃)—CH₃ |
| A-751 | CH(CH₃)₂ | CH₂—CH(CH═CH₂)—CH(CH₃)—CH₃ |
| A-752 | CH(CH₃)₂ | CH₂—C(CH₂—CH₃)═C(CH₃)—CH₃ |
| A-753 | CH(CH₃)₂ | CH₂—CH(CH₂—CH₃)—C(═CH₂)—CH₃ |
| A-754 | CH(CH₃)₂ | CH₂—C(CH₃)(CH═CH₂)—CH₂—CH₃ |
| A-755 | CH(CH₃)₂ | C(═CH₂)—CH(CH₂—CH₃)—CH₂—CH₃ |
| A-756 | CH(CH₃)₂ | C(CH₃)═C(CH₂—CH₃)—CH₂—CH₃ |
| A-757 | CH(CH₃)₂ | CH(CH₃)—C(═CH—CH₃)—CH₂—CH₃ |
| A-758 | CH(CH₃)₂ | CH(CH₃)—CH(CH═CH₂)—CH₂—CH₃ |
| A-759 | CH(CH₃)₂ | CH═C(CH₂—CH₃)—CH(CH₃)—CH₃ |
| A-760 | CH(CH₃)₂ | CH₂—C(═CH—CH₃)—CH(CH₃)—CH₃ |
| A-761 | CH(CH₃)₂ | CH₂—CH(CH═CH₂)—CH(CH₃)—CH₃ |
| A-762 | CH(CH₃)₂ | CH₂—C(CH₂—CH₃)═C(CH₃)—CH₃ |
| A-763 | CH(CH₃)₂ | CH₂—CH(CH₂—CH₃)—C(═CH₂)—CH₃ |
| A-764 | CH(CH₃)₂ | C(═CH—CH₃)—CH₂—CH(CH₃)—CH₃ |
| A-765 | CH(CH₃)₂ | CH(CH═CH₂)—CH₂—CH(CH₃)—CH₃ |
| A-766 | CH(CH₃)₂ | C(CH₂—CH₃)═CH—CH(CH₃)—CH₃ |
| A-767 | CH(CH₃)₂ | CH(CH₂—CH₃)CH═C(CH₃)—CH₃ |
| A-768 | CH(CH₃)₂ | CH(CH₂—CH₃)CH₂—C(═CH₂)—CH₃ |
| A-769 | CH(CH₃)₂ | C(═CH—CH₃)CH(CH₃)—CH₂—CH₃ |
| A-770 | CH(CH₃)₂ | CH(CH═CH₂)CH(CH₃)—CH₂—CH₃ |
| A-771 | CH(CH₃)₂ | C(CH₂—CH₃)═C(CH₃)—CH₂—CH₃ |
| A-772 | CH(CH₃)₂ | CH(CH₂—CH₃)—C(═CH₂)—CH₂—CH₃ |
| A-773 | CH(CH₃)₂ | CH(CH₂—CH₃)—C(CH₃)═CH—CH₃ |
| A-774 | CH(CH₃)₂ | CH(CH₂—CH₃)—CH(CH₃)—CH═CH₂ |
| A-775 | CH(CH₃)₂ | C(CH₃)(CH═CH₂)—CH₂—CH₂—CH₃ |
| A-776 | CH(CH₃)₂ | C(CH₃)(CH₂—CH₃)—CH═CH—CH₃ |
| A-777 | CH(CH₃)₂ | C(CH₃)(CH₂—CH₃)—CH₂—CH═CH₂ |
| A-778 | CH(CH₃)₂ | C[═C(CH₃)—CH₃]—CH₂—CH₂—CH₃ |
| A-779 | CH(CH₃)₂ | CH[C(═CH₂)—CH₃]—CH₂—CH₂—CH₃ |
| A-780 | CH(CH₃)₂ | C(i-C₃H₇)═CH—CH₂—CH₃ |
| A-781 | CH(CH₃)₂ | CH(i-C₃H₇)—CH═CH—CH₃ |
| A-782 | CH(CH₃)₂ | CH(i-C₃H₇)—CH₂—CH═CH₂ |
| A-783 | CH(CH₃)₂ | C(═CH—CH₃)—C(CH₃)₃ |
| A-784 | CH(CH₃)₂ | CH(CH═CH₂)—C(CH₃)₃ |
| A-785 | CH(CH₃)₂ | C(CH₃)(CH═CH₂)CH(CH₃)—CH₃ |
| A-786 | CH(CH₃)₂ | C(CH₃)(CH₂—CH₃)C(═CH₂)—CH₃ |
| A-787 | CH(CH₃)₂ | 2-CH₃-cyclohex-1-enyl |
| A-788 | CH(CH₃)₂ | [2-(═CH₂)]-cyclo-C₆H₉ |
| A-789 | CH(CH₃)₂ | 2-CH₃-cyclohex-2-enyl |
| A-790 | CH(CH₃)₂ | 2-CH₃-cyclohex-3-enyl |
| A-791 | CH(CH₃)₂ | 2-CH₃-cyclohex-4-enyl |
| A-792 | CH(CH₃)₂ | 2-CH₃-cyclohex-5-enyl |
| A-793 | CH(CH₃)₂ | 2-CH₃-cyclohex-6-enyl |
| A-794 | CH(CH₃)₂ | 3-CH₃-cyclohex-1-enyl |
| A-795 | CH(CH₃)₂ | 3-CH₃-cyclohex-2-enyl |
| A-796 | CH(CH₃)₂ | [3-(═CH₂)]-cyclo-C₆H₉ |
| A-797 | CH(CH₃)₂ | 3-CH₃-cyclohex-3-enyl |
| A-798 | CH(CH₃)₂ | 3-CH₃-cyclohex-4-enyl |
| A-799 | CH(CH₃)₂ | 3-CH₃-cyclohex-5-enyl |
| A-800 | CH(CH₃)₂ | 3-CH₃-cyclohex-6-enyl |
| A-801 | CH(CH₃)₂ | 4-CH₃-cyclohex-1-enyl |
| A-802 | CH(CH₃)₂ | 4-CH₃-cyclohex-2-enyl |
| A-803 | CH(CH₃)₂ | 4-CH₃-cyclohex-3-enyl |
| A-804 | CH(CH₃)₂ | [4-(═CH₂)]-cyclo-C₆H₉ |
| A-805 | CH(CH₃)₂ | CH₂CF₃ |
| A-806 | CH(CH₃)₂ | CH₂CCl₃ |
| A-807 | CH(CH₃)₂ | CH₂(cyclo-C₃H₅) |
| A-808 | CH₂(cyclo-C₃H₅) | CH₂CH₃ |
| A-809 | CH₂(cyclo-C₃H₅) | CH₂CH₂CH₃ |
| A-810 | CH₂(cyclo-C₃H₅) | CH₂CH₂F |
| A-811 | CH₂(cyclo-C₃H₅) | H |
| A-812 | CH₂(cyclo-C₃H₅) | CH(CH₃)₂ |
| A-813 | CH₂(cyclo-C₃H₅) | CH₂C(CH₃)₃ |
| A-814 | CH₂(cyclo-C₃H₅) | CH₂CH(CH₃)₂ |
| A-815 | CH₂(cyclo-C₃H₅) | (±)CH(CH₂CH₃)CH₃ |
| A-816 | CH₂(cyclo-C₃H₅) | (R)CH(CH₂CH₃)CH₃ |
| A-817 | CH₂(cyclo-C₃H₅) | (S)CH(CH₂CH₃)CH₃ |
| A-818 | CH₂(cyclo-C₃H₅) | (±)CH(CH₃)—CH(CH₃)₂ |
| A-819 | CH₂(cyclo-C₃H₅) | (R)CH(CH₃)—CH(CH₃)₂ |
| A-820 | CH₂(cyclo-C₃H₅) | (S)CH(CH₃)—CH(CH₃)₂ |
| A-821 | CH₂(cyclo-C₃H₅) | (±)CH(CH₃)—C(CH₃)₃ |
| A-822 | CH₂(cyclo-C₃H₅) | (R)CH(CH₃)—C(CH₃)₃ |
| A-823 | CH₂(cyclo-C₃H₅) | (S)CH(CH₃)—C(CH₃)₃ |
| A-824 | CH₂(cyclo-C₃H₅) | (±)CH(CH₃)—CF₃ |
| A-825 | CH₂(cyclo-C₃H₅) | (R)CH(CH₃)—CF₃ |
| A-826 | CH₂(cyclo-C₃H₅) | (S)CH(CH₃)—CF₃ |
| A-827 | CH₂(cyclo-C₃H₅) | (±)CH(CH₃)—CCl₃ |
| A-828 | CH₂(cyclo-C₃H₅) | (R)CH(CH₃)—CCl₃ |
| A-829 | CH₂(cyclo-C₃H₅) | (S)CH(CH₃)—CCl₃ |
| A-830 | CH₂(cyclo-C₃H₅) | CH₂C(CH₃)═CH₂ |
| A-831 | CH₂(cyclo-C₃H₅) | cyclopentyl |
| A-832 | CH₂(cyclo-C₃H₅) | cyclohexyl |
| A-833 | CH₂(cyclo-C₃H₅) | (CH₂)₃CH₃ |
| A-834 | CH₂(cyclo-C₃H₅) | C(CH₃)₃ |
| A-835 | CH₂(cyclo-C₃H₅) | (CH₂)₄CH₃ |
| A-836 | CH₂(cyclo-C₃H₅) | CH(CH₂CH₃)₂ |
| A-837 | CH₂(cyclo-C₃H₅) | CH₂CH₂CH(CH₃)₂ |
| A-838 | CH₂(cyclo-C₃H₅) | (±)CH(CH₃)(CH₂)₂CH₃ |
| A-839 | CH₂(cyclo-C₃H₅) | (R)CH(CH₃)(CH₂)₂CH₃ |
| A-840 | CH₂(cyclo-C₃H₅) | (S)CH(CH₃)(CH₂)₂CH₃ |
| A-841 | CH₂(cyclo-C₃H₅) | (±)CH₂CH(CH₃)CH₂CH₃ |
| A-842 | CH₂(cyclo-C₃H₅) | (R)CH₂CH(CH₃)CH₂CH₃ |
| A-843 | CH₂(cyclo-C₃H₅) | (S)CH₂CH(CH₃)CH₂CH₃ |
| A-844 | CH₂(cyclo-C₃H₅) | (±)CH(CH₃)CH(CH₃)₂ |
| A-845 | CH₂(cyclo-C₃H₅) | (R)CH(CH₃)CH(CH₃)₂ |
| A-846 | CH₂(cyclo-C₃H₅) | (S)CH(CH₃)CH(CH₃)₂ |
| A-847 | CH₂(cyclo-C₃H₅) | (CH₂)₅CH₃ |
| A-848 | CH₂(cyclo-C₃H₅) | (±,±)CH(CH₃)CH(CH₃)CH₂CH₃ |
| A-849 | CH₂(cyclo-C₃H₅) | (±,R)CH(CH₃)CH(CH₃)CH₂CH₃ |
| A-850 | CH₂(cyclo-C₃H₅) | (±,S)CH(CH₃)CH(CH₃)CH₂CH₃ |
| A-851 | CH₂(cyclo-C₃H₅) | (±)CH₂CH(CH₃)CF₃ |
| A-852 | CH₂(cyclo-C₃H₅) | (R)CH₂CH(CH₃)CF₃ |
| A-853 | CH₂(cyclo-C₃H₅) | (S)CH₂CH(CH₃)CF₃ |
| A-854 | CH₂(cyclo-C₃H₅) | (±)CH₂CH(CF₃)CH₂CH₃ |
| A-855 | CH₂(cyclo-C₃H₅) | (R)CH₂CH(CF₃)CH₂CH₃ |
| A-856 | CH₂(cyclo-C₃H₅) | (S)CH₂CH(CF₃)CH₂CH₃ |
| A-857 | CH₂(cyclo-C₃H₅) | (±,±)CH(CH₃)CH(CH₃)CF₃ |
| A-858 | CH₂(cyclo-C₃H₅) | (±,R)CH(CH₃)CH(CH₃)CF₃ |
| A-859 | CH₂(cyclo-C₃H₅) | (±,S)CH(CH₃)CH(CH₃)CF₃ |
| A-860 | CH₂(cyclo-C₃H₅) | (±,±)CH(CH₃)CH(CF₃)CH₂CH₃ |
| A-861 | CH₂(cyclo-C₃H₅) | (±,R)CH(CH₃)CH(CF₃)CH₂CH₃ |
| A-862 | CH₂(cyclo-C₃H₅) | (±,S)CH(CH₃)CH(CF₃)CH₂CH₃ |
| A-863 | CH₂(cyclo-C₃H₅) | CF₃ |
| A-864 | CH₂(cyclo-C₃H₅) | CF₂CF₃ |
| A-865 | CH₂(cyclo-C₃H₅) | CF₂CF₂CF₃ |
| A-866 | CH₂(cyclo-C₃H₅) | cyclo-C₃H₅ |
| A-867 | CH₂(cyclo-C₃H₅) | (1-CH₃)-cyclo-C₃H₄ |
| A-868 | CH₂(cyclo-C₃H₅) | cyclo-C₅H₉ |
| A-869 | CH₂(cyclo-C₃H₅) | cyclo-C₆H₁₁ |
| A-870 | CH₂(cyclo-C₃H₅) | (4-CH₃)-cyclo-C₆H₁₀ |
| A-871 | CH₂(cyclo-C₃H₅) | CH₂C(CH₃)═CH₂ |
| A-872 | CH₂(cyclo-C₃H₅) | CH₂CH₂C(CH₃)═CH₂ |
| A-873 | CH₂(cyclo-C₃H₅) | CH₂—C(CH₃)₃ |
| A-874 | CH₂(cyclo-C₃H₅) | CH₂—Si(CH₃)₃ |
| A-875 | CH₂(cyclo-C₃H₅) | n-C₆H₁₃ |
| A-876 | CH₂(cyclo-C₃H₅) | (CH₂)₃—CH(CH₃)₂ |
| A-877 | CH₂(cyclo-C₃H₅) | (CH₂)₂—CH(CH₃)—C₂H₅ |
| A-878 | CH₂(cyclo-C₃H₅) | CH₂—CH(CH₃)-n-C₃H₇ |
| A-879 | CH₂(cyclo-C₃H₅) | CH(CH₃)-n-C₄H₉ |
| A-880 | CH₂(cyclo-C₃H₅) | CH₂—CH(C₂H₅)₂ |
| A-881 | CH₂(cyclo-C₃H₅) | CH(C₂H₅)-n-C₃H₇ |
| A-882 | CH₂(cyclo-C₃H₅) | CH₂-cyclo-C₅H₉ |
| A-883 | CH₂(cyclo-C₃H₅) | CH₂—CH(CH₃)—CH(CH₃)₂ |
| A-884 | CH₂(cyclo-C₃H₅) | CH(CH₃)—CH₂CH(CH₃)₂ |
| A-885 | CH₂(cyclo-C₃H₅) | CH(CH₃)—CH(CH₃)—C₂H₅ |
| A-886 | CH₂(cyclo-C₃H₅) | CH(CH₃)—C(CH₃)₃ |
| A-887 | CH₂(cyclo-C₃H₅) | (CH₂)₂—C(CH₃)₃ |
| A-888 | CH₂(cyclo-C₃H₅) | CH₂—C(CH₃)₂—C₂H₅ |
| A-889 | CH₂(cyclo-C₃H₅) | 2-CH₃-cyclo-C₅H₈ |
| A-890 | CH₂(cyclo-C₃H₅) | 3-CH₃-cyclo-C₅H₈ |
| A-891 | CH₂(cyclo-C₃H₅) | C(CH₃)₂-n-C₃H₇ |
| A-892 | CH₂(cyclo-C₃H₅) | (CH₂)₆—CH₃ |
| A-893 | CH₂(cyclo-C₃H₅) | (CH₂)₄—CH(CH₃)₂ |
| A-894 | CH₂(cyclo-C₃H₅) | (CH₂)₃—CH(CH₃)—C₂H₅ |
| A-895 | CH₂(cyclo-C₃H₅) | (CH₂)₂—CH(CH₃)-n-C₃H₇ |
| A-896 | CH₂(cyclo-C₃H₅) | CH₂—CH(CH₃)-n-C₄H₉ |
| A-897 | CH₂(cyclo-C₃H₅) | CH(CH₃)-n-C₅H₁₁ |
| A-898 | CH₂(cyclo-C₃H₅) | (CH₂)₃C(CH₃)₃ |
| A-899 | CH₂(cyclo-C₃H₅) | (CH₂)₂CH(CH₃)—CH(CH₃)₂ |
| A-900 | CH₂(cyclo-C₃H₅) | (CH₂)CH(CH₃)—CH₂CH(CH₃)₂ |
| A-901 | CH₂(cyclo-C₃H₅) | CH(CH₃)(CH₂)₂—CH(CH₃)₂ |
| A-902 | CH₂(cyclo-C₃H₅) | (CH₂)₂C(CH₃)₂C₂H₅ |
| A-903 | CH₂(cyclo-C₃H₅) | CH₂CH(CH₃)CH(CH₃)C₂H₅ |
| A-904 | CH₂(cyclo-C₃H₅) | CH(CH₃)CH₂CH(CH₃)C₂H₅ |
| A-905 | CH₂(cyclo-C₃H₅) | CH₂C(CH₃)₂-n-C₃H₇ |
| A-906 | CH₂(cyclo-C₃H₅) | CH(CH₃)CH(CH₃)-n-C₃H₇ |
| A-907 | CH₂(cyclo-C₃H₅) | C(CH₃)₂-n-C₄H₉ |
| A-908 | CH₂(cyclo-C₃H₅) | (CH₂)₂CH(C₂H₅)₂ |
| A-909 | CH₂(cyclo-C₃H₅) | CH₂CH(C₂H₅)-n-C₃H₇ |
| A-910 | CH₂(cyclo-C₃H₅) | CH(C₂H₅)-n-C₄H₉ |
| A-911 | CH₂(cyclo-C₃H₅) | CH₂CH(CH₃)C(CH₃)₃ |
| A-912 | CH₂(cyclo-C₃H₅) | CH(CH₃)CH₂C(CH₃)₃ |
| A-913 | CH₂(cyclo-C₃H₅) | CH₂C(CH₃)₂CH(CH₃)₂ |
| A-914 | CH₂(cyclo-C₃H₅) | CH₂CH(C₂H₅)CH(CH₃)₂ |
| A-915 | CH₂(cyclo-C₃H₅) | CH(CH₃)CH(CH₃)CH(CH₃)₂ |
| A-916 | CH₂(cyclo-C₃H₅) | C(CH₃)₂CH₂CH(CH₃)₂ |
| A-917 | CH₂(cyclo-C₃H₅) | CH(C₂H₅)CH₂CH(CH₃)₂ |
| A-918 | CH₂(cyclo-C₃H₅) | CH(CH₃)C(CH₃)₂C₂H₅ |
| A-919 | CH₂(cyclo-C₃H₅) | CH(CH₃)CH(C₂H₅)₂ |
| A-920 | CH₂(cyclo-C₃H₅) | C(CH₃)₂CH(CH₃)C₂H₅ |
| A-921 | CH₂(cyclo-C₃H₅) | CH(C₂H₅)CH(CH₃)C₂H₅ |
| A-922 | CH₂(cyclo-C₃H₅) | C(CH₃)(C₂H₅)-n-C₃H₇ |
| A-923 | CH₂(cyclo-C₃H₅) | CH(n-C₃H₇)₂ |
| A-924 | CH₂(cyclo-C₃H₅) | CH(n-C₃H₇)CH(CH₃)₂ |
| A-925 | CH₂(cyclo-C₃H₅) | C(CH₃)₂C(CH₃)₃ |
| A-926 | CH₂(cyclo-C₃H₅) | C(CH₃)(C₂H₅)—CH(CH₃)₂ |
| A-927 | CH₂(cyclo-C₃H₅) | C(C₂H₅)₃ |
| A-928 | CH₂(cyclo-C₃H₅) | (3-CH₃)-cyclo-C₆H₁₀ |
| A-929 | CH₂(cyclo-C₃H₅) | (2-CH₃)-cyclo-C₆H₁₀ |
| A-930 | CH₂(cyclo-C₃H₅) | n-C₈H₁₇ |
| A-931 | CH₂(cyclo-C₃H₅) | CH₂C(═NO—CH₃)CH₃ |
| A-932 | CH₂(cyclo-C₃H₅) | CH₂C(═NO—C₂H₅)CH₃ |
| A-933 | CH₂(cyclo-C₃H₅) | CH₂C(═NO-n-C₃H₇)CH₃ |
| A-934 | CH₂(cyclo-C₃H₅) | CH₂C(═NO-i-C₃H₇)CH₃ |
| A-935 | CH₂(cyclo-C₃H₅) | CH(CH₃)C(═NOCH₃)CH₃ |
| A-936 | CH₂(cyclo-C₃H₅) | CH(CH₃)C(═NOC₂H₅)CH₃ |
| A-937 | CH₂(cyclo-C₃H₅) | CH(CH₃)C(═NO-n-C₃H₇)CH₃ |
| A-938 | CH₂(cyclo-C₃H₅) | CH(CH₃)C(═NO-i-C₃H₇)CH₃ |
| A-939 | CH₂(cyclo-C₃H₅) | CH₂C(═NO—CH₃)C₂H₅ |
| A-940 | CH₂(cyclo-C₃H₅) | CH₂C(═NO—C₂H₅)C₂H₅ |
| A-941 | CH₂(cyclo-C₃H₅) | CH₂C(═NO-n-C₃H₇)C₂H₅ |
| A-942 | CH₂(cyclo-C₃H₅) | CH₂C(═NO-i-C₃H₇)C₂H₅ |
| A-943 | CH₂(cyclo-C₃H₅) | CH(CH₃)C(═NOCH₃)C₂H₅ |
| A-944 | CH₂(cyclo-C₃H₅) | CH(CH₃)C(═NOC₂H₅)C₂H₅ |
| A-945 | CH₂(cyclo-C₃H₅) | CH(CH₃)C(═NO-n-C₃H₇)C₂H₅ |
| A-946 | CH₂(cyclo-C₃H₅) | CH(CH₃)C(═NO-n-C₃H₇)C₂H₅ |
| A-947 | CH₂(cyclo-C₃H₅) | CH═CH—CH₂CH₃ |
| A-948 | CH₂(cyclo-C₃H₅) | CH₂—CH═CH—CH₃ |
| A-949 | CH₂(cyclo-C₃H₅) | CH₂—CH₂—CH═CH₂ |
| A-950 | CH₂(cyclo-C₃H₅) | C(CH₃)₂CH₂CH₃ |
| A-951 | CH₂(cyclo-C₃H₅) | CH═C(CH₃)₂ |
| A-952 | CH₂(cyclo-C₃H₅) | C(═CH₂)—CH₂CH₃ |
| A-953 | CH₂(cyclo-C₃H₅) | C(CH₃)═CH—CH₃ |
| A-954 | CH₂(cyclo-C₃H₅) | CH(CH₃)CH═CH₂ |
| A-955 | CH₂(cyclo-C₃H₅) | CH═CH-n-C₃H₇ |
| A-956 | CH₂(cyclo-C₃H₅) | CH₂—CH═CH—C₂H₅ |
| A-957 | CH₂(cyclo-C₃H₅) | (CH₂)₂—CH═CH—CH₃ |
| A-958 | CH₂(cyclo-C₃H₅) | (CH₂)₃—CH═CH₂ |
| A-959 | CH₂(cyclo-C₃H₅) | CH═CH—CH(CH₃)₂ |
| A-960 | CH₂(cyclo-C₃H₅) | CH₂—CH═C(CH₃)₂ |
| A-961 | CH₂(cyclo-C₃H₅) | (CH₂)₂—C(CH₃)═CH₂ |
| A-962 | CH₂(cyclo-C₃H₅) | CH═C(CH₃)—C₂H₅ |
| A-963 | CH₂(cyclo-C₃H₅) | CH₂—C(═CH₂)—C₂H₅ |
| A-964 | CH₂(cyclo-C₃H₅) | CH₂—C(CH₃)═CH—CH₃ |
| A-965 | CH₂(cyclo-C₃H₅) | CH₂—CH(CH₃)—CH═CH₂ |
| A-966 | CH₂(cyclo-C₃H₅) | C(═CH₂)—CH₂—CH₂—CH₃ |
| A-967 | CH₂(cyclo-C₃H₅) | C(CH₃)═CH—CH₂—CH₃ |
| A-968 | CH₂(cyclo-C₃H₅) | CH(CH₃)—CH═CH—CH₃ |
| A-969 | CH₂(cyclo-C₃H₅) | CH(CH₃)—CH₂—CH═CH₂ |
| A-970 | CH₂(cyclo-C₃H₅) | C(═CH₂)CH(CH₃)₂ |
| A-971 | CH₂(cyclo-C₃H₅) | C(CH₃)═C(CH₃)₂ |
| A-972 | CH₂(cyclo-C₃H₅) | CH(CH₃)—C(═CH₂)—CH₃ |
| A-973 | CH₂(cyclo-C₃H₅) | C(CH₃)₂—CH═CH₂ |
| A-974 | CH₂(cyclo-C₃H₅) | C(C₂H₅)═CH—CH₃ |
| A-975 | CH₂(cyclo-C₃H₅) | CH(C₂H₅)—CH═CH₂ |
| A-976 | CH₂(cyclo-C₃H₅) | CH═CH—CH₂—CH₂—CH₂—CH₃ |
| A-977 | CH₂(cyclo-C₃H₅) | CH₂—CH═CH—CH₂—CH₂—CH₃ |
| A-978 | CH₂(cyclo-C₃H₅) | CH₂—CH₂—CH═CH—CH₂—CH₃ |
| A-979 | CH₂(cyclo-C₃H₅) | CH₂—CH₂—CH₂—CH═CH—CH₃ |
| A-980 | CH₂(cyclo-C₃H₅) | CH₂—CH₂—CH₂—CH₂—CH═CH₂ |
| A-981 | CH₂(cyclo-C₃H₅) | CH═CH—CH₂—CH(CH₃)CH₃ |
| A-982 | CH₂(cyclo-C₃H₅) | CH₂—CH═CH—CH(CH₃)CH₃ |
| A-983 | CH₂(cyclo-C₃H₅) | CH₂—CH₂—CH═C(CH₃)CH₃ |
| A-984 | CH₂(cyclo-C₃H₅) | CH₂—CH₂—CH₂—C(CH₃)═CH₂ |
| A-985 | CH₂(cyclo-C₃H₅) | CH═CH—CH(CH₃)—CH₂—CH₃ |
| A-986 | CH₂(cyclo-C₃H₅) | CH₂—CH═C(CH₃)—CH₂—CH₃ |
| A-987 | CH₂(cyclo-C₃H₅) | CH₂—CH₂—C(═CH₂)—CH₂—CH₃ |
| A-988 | CH₂(cyclo-C₃H₅) | CH₂—CH₂—C(CH₃)═CH—CH₃ |
| A-989 | CH₂(cyclo-C₃H₅) | CH₂—CH₂—CH(CH₃)—CH═CH₂ |
| A-990 | CH₂(cyclo-C₃H₅) | CH═C(CH₃)—CH₂—CH₂—CH₃ |
| A-991 | CH₂(cyclo-C₃H₅) | CH₂—C(═CH₂)—CH₂—CH₂—CH₃ |
| A-992 | CH₂(cyclo-C₃H₅) | CH₂—C(CH₃)═CH—CH₂—CH₃ |
| A-993 | CH₂(cyclo-C₃H₅) | CH₂—CH(CH₃)—CH═CH—CH₃ |
| A-994 | CH₂(cyclo-C₃H₅) | CH₂—CH(CH₃)—CH₂—CH═CH₂ |
| A-995 | CH₂(cyclo-C₃H₅) | C(═CH₂)—CH₂—CH₂—CH₂—CH₃ |
| A-996 | CH₂(cyclo-C₃H₅) | C(CH₃)═CH—CH₂—CH₂—CH₃ |
| A-997 | CH₂(cyclo-C₃H₅) | CH(CH₃)—CH═CH—CH₂—CH₃ |
| A-998 | CH₂(cyclo-C₃H₅) | CH(CH₃)—CH₂—CH═CH—CH₃ |
| A-999 | CH₂(cyclo-C₃H₅) | CH(CH₃)—CH₂—CH₂—CH═CH₂ |
| A-1000 | CH₂(cyclo-C₃H₅) | CH═CH—C(CH₃)₃ |
| A-1001 | CH₂(cyclo-C₃H₅) | CH═C(CH₃)—CH(CH₃)—CH₃ |
| A-1002 | CH₂(cyclo-C₃H₅) | CH₂—C(═CH₂)—CH(CH₃)—CH₃ |
| A-1003 | CH₂(cyclo-C₃H₅) | CH₂—C(CH₃)═C(CH₃)—CH₃ |
| A-1004 | CH₂(cyclo-C₃H₅) | CH₂—CH(CH₃)—C(═CH₂)—CH₃ |
| A-1005 | CH₂(cyclo-C₃H₅) | C(═CH₂)—CH₂—CH(CH₃)—CH₃ |
| A-1006 | CH₂(cyclo-C₃H₅) | C(CH₃)═CH—CH(CH₃)—CH₃ |
| A-1007 | CH₂(cyclo-C₃H₅) | CH(CH₃)—CH═C(CH₃)—CH₃ |
| A-1008 | CH₂(cyclo-C₃H₅) | CH(CH₃)—CH₂—C(═CH₂)—CH₃ |
| A-1009 | CH₂(cyclo-C₃H₅) | CH═C(CH₂—CH₃)—CH₂—CH₃ |
| A-1010 | CH₂(cyclo-C₃H₅) | CH₂—C(═CH—CH₃)—CH₂—CH₃ |
| A-1011 | CH₂(cyclo-C₃H₅) | CH₂—CH(CH═CH₂)—CH₂—CH₃ |
| A-1012 | CH₂(cyclo-C₃H₅) | C(═CH—CH₃)—CH₂—CH₂—CH₃ |
| A-1013 | CH₂(cyclo-C₃H₅) | CH(CH═CH₂)—CH₂—CH₂—CH₃ |
| A-1014 | CH₂(cyclo-C₃H₅) | C(CH₂—CH₃)═CH—CH₂—CH₃ |
| A-1015 | CH₂(cyclo-C₃H₅) | CH(CH₂—CH₃)—CH═CH—CH₃ |
| A-1016 | CH₂(cyclo-C₃H₅) | CH(CH₂—CH₃)—CH₂—CH═CH₂ |
| A-1017 | CH₂(cyclo-C₃H₅) | CH₂—C(CH₃)₂—CH═CH₂ |
| A-1018 | CH₂(cyclo-C₃H₅) | C(═CH₂)—CH(CH₃)—CH₂—CH₃ |
| A-1019 | CH₂(cyclo-C₃H₅) | C(CH₃)═C(CH₃)—CH₂—CH₃ |
| A-1020 | CH₂(cyclo-C₃H₅) | CH(CH₃)—C(═CH₂)—CH₂—CH₃ |
| A-1021 | CH₂(cyclo-C₃H₅) | CH(CH₃)—C(CH₃)═CH—CH₃ |
| A-1022 | CH₂(cyclo-C₃H₅) | CH(CH₃)—CH(CH₃)—CH═CH₂ |
| A-1023 | CH₂(cyclo-C₃H₅) | C(CH₃)₂—CH═CH—CH₃ |
| A-1024 | CH₂(cyclo-C₃H₅) | C(CH₃)₂—CH₂—CH═CH₂ |
| A-1025 | CH₂(cyclo-C₃H₅) | C(═CH₂)—C(CH₃)₃ |
| A-1026 | CH₂(cyclo-C₃H₅) | C(═CH—CH₃)—CH(CH₃)—CH₃ |
| A-1027 | CH₂(cyclo-C₃H₅) | CH(CH═CH₂)—CH(CH₃)—CH₃ |
| A-1028 | CH₂(cyclo-C₃H₅) | C(CH₂—CH₃)═C(CH₃)—CH₃ |
| A-1029 | CH₂(cyclo-C₃H₅) | CH(CH₂—CH₃)—C(═CH₂)—CH₃ |
| A-1030 | CH₂(cyclo-C₃H₅) | C(CH₃)₂—C(═CH₂)—CH₃ |
| A-1031 | CH₂(cyclo-C₃H₅) | C(CH₃)(CH═CH₂)—CH₂—CH₃ |
| A-1032 | CH₂(cyclo-C₃H₅) | C(CH₃)(CH₂CH₃)—CH₂—CH₂—CH₃ |
| A-1033 | CH₂(cyclo-C₃H₅) | CH(CH₂CH₃)—CH(CH₃)—CH₂—CH₃ |
| A-1034 | CH₂(cyclo-C₃H₅) | CH(CH₂CH₃)—CH₂—CH(CH₃)—CH₃ |
| A-1035 | CH₂(cyclo-C₃H₅) | C(CH₃)₂—C(CH₃)₃ |
| A-1036 | CH₂(cyclo-C₃H₅) | C(CH₂—CH₃)—C(CH₃)₃ |
| A-1037 | CH₂(cyclo-C₃H₅) | C(CH₃)(CH₂—CH₃)—CH(CH₃)₂ |
| A-1038 | CH₂(cyclo-C₃H₅) | CH(CH(CH₃)₂)—CH(CH₃)₂ |
| A-1039 | CH₂(cyclo-C₃H₅) | CH═CH—CH₂—CH₂—CH₂—CH₂—CH₃ |
| A-1040 | CH₂(cyclo-C₃H₅) | CH₂—CH═CH—CH₂—CH₂—CH₂—CH₃ |
| A-1041 | CH₂(cyclo-C₃H₅) | CH₂—CH₂—CH═CH—CH₂—CH₂—CH₃ |
| A-1042 | CH₂(cyclo-C₃H₅) | CH₂—CH₂—CH₂—CH═CH—CH₂—CH₃ |
| A-1043 | CH₂(cyclo-C₃H₅) | CH₂—CH₂—CH₂—CH₂—CH═CH—CH₃ |
| A-1044 | CH₂(cyclo-C₃H₅) | CH₂—CH₂—CH₂—CH₂—CH₂—CH═CH₂ |
| A-1045 | CH₂(cyclo-C₃H₅) | CH═CH—CH₂—CH₂—CH(CH₃)—CH₃ |
| A-1046 | CH₂(cyclo-C₃H₅) | CH₂—CH═CH—CH₂—CH(CH₃)—CH₃ |
| A-1047 | CH₂(cyclo-C₃H₅) | CH₂—CH₂—CH═CH—CH(CH₃)—CH₃ |
| A-1048 | CH₂(cyclo-C₃H₅) | CH₂—CH₂—CH₂—CH═C(CH₃)—CH₃ |
| A-1049 | CH₂(cyclo-C₃H₅) | CH₂—CH₂—CH₂—CH₂—C(═CH₂)—CH₃ |
| A-1050 | CH₂(cyclo-C₃H₅) | CH═CH—CH₂—CH(CH₃)—CH₂—CH₃ |
| A-1051 | CH₂(cyclo-C₃H₅) | CH₂—CH═CH—CH(CH₃)—CH₂—CH₃ |
| A-1052 | CH₂(cyclo-C₃H₅) | CH₂—CH₂—CH═C(CH₃)—CH₂—CH₃ |
| A-1053 | CH₂(cyclo-C₃H₅) | CH₂—CH₂—CH₂—C(═CH₂)—CH₂—CH₃ |
| A-1054 | CH₂(cyclo-C₃H₅) | CH₂—CH₂—CH₂—C(CH₃)═CH—CH₃ |
| A-1055 | CH₂(cyclo-C₃H₅) | CH₂—CH₂—CH₂—CH(CH₃)—CH═CH₂ |
| A-1056 | CH₂(cyclo-C₃H₅) | CH═CH—CH(CH₃)—CH₂—CH₂—CH₃ |
| A-1057 | CH₂(cyclo-C₃H₅) | CH₂—CH═C(CH₃)—CH₂—CH₂—CH₃ |
| A-1058 | CH₂(cyclo-C₃H₅) | CH₂—CH₂—C(═CH₂)—CH₂—CH₂—CH₃ |
| A-1059 | CH₂(cyclo-C₃H₅) | CH₂—CH₂—C(CH₃)═CH—CH₂—CH₃ |
| A-1060 | CH₂(cyclo-C₃H₅) | CH₂—CH₂—CH(CH₃)—CH═CH—CH₃ |
| A-1061 | CH₂(cyclo-C₃H₅) | CH₂—CH₂—CH(CH₃)—CH₂—CH═CH₂ |
| A-1062 | CH₂(cyclo-C₃H₅) | CH═C(CH₃)—CH₂—CH₂—CH₂—CH₃ |
| A-1063 | CH₂(cyclo-C₃H₅) | CH₂—C(═CH₂)—CH₂—CH₂—CH₂—CH₃ |
| A-1064 | CH₂(cyclo-C₃H₅) | CH₂—C(CH₃)═CH—CH₂—CH₂—CH₃ |
| A-1065 | CH₂(cyclo-C₃H₅) | CH₂—CH(CH₃)—CH═CH—CH₂—CH₃ |
| A-1066 | CH₂(cyclo-C₃H₅) | CH₂—CH(CH₃)—CH₂—CH═CH—CH₃ |
| A-1067 | CH₂(cyclo-C₃H₅) | CH₂—CH(CH₃)—CH₂—CH₂—CH═CH₂ |
| A-1068 | CH₂(cyclo-C₃H₅) | C(═CH₂)—CH₂—CH₂—CH₂—CH₂—CH₃ |
| A-1069 | CH₂(cyclo-C₃H₅) | C(CH₃)═CH—CH₂—CH₂—CH₂—CH₃ |
| A-1070 | CH₂(cyclo-C₃H₅) | CH(CH₃)—CH═CH—CH₂—CH₂—CH₃ |
| A-1071 | CH₂(cyclo-C₃H₅) | CH(CH₃)—CH₂—CH═CH—CH₂—CH₃ |
| A-1072 | CH₂(cyclo-C₃H₅) | CH(CH₃)—CH₂—CH₂—CH═CH—CH₃ |
| A-1073 | CH₂(cyclo-C₃H₅) | CH(CH₃)—CH₂—CH₂—CH₂—CH═CH₂ |
| A-1074 | CH₂(cyclo-C₃H₅) | CH═CH—CH₂—C(CH₃)₃ |
| A-1075 | CH₂(cyclo-C₃H₅) | CH₂—CH═CH—C(CH₃)₃ |
| A-1076 | CH₂(cyclo-C₃H₅) | CH═CH—CH(CH₃)—CH(CH₃)₂ |
| A-1077 | CH₂(cyclo-C₃H₅) | CH₂—CH═C(CH₃)—CH(CH₃)₂ |
| A-1078 | CH₂(cyclo-C₃H₅) | CH₂—CH₂—C(═CH₂)—CH(CH₃)₂ |
| A-1079 | CH₂(cyclo-C₃H₅) | CH₂—CH₂—C(CH₃)═C(CH₃)₂ |
| A-1080 | CH₂(cyclo-C₃H₅) | CH₂—CH₂—CH(CH₃)—C(═CH₂)—CH₃ |
| A-1081 | CH₂(cyclo-C₃H₅) | CH═C(CH₃)—CH₂—CH(CH₃)₂ |
| A-1082 | CH₂(cyclo-C₃H₅) | CH₂—C(═CH₂)—CH₂—CH(CH₃)₂ |
| A-1083 | CH₂(cyclo-C₃H₅) | CH₂—C(CH₃)═CH—CH(CH₃)₂ |
| A-1084 | CH₂(cyclo-C₃H₅) | CH₂—CH(CH₃)—CH═C(CH₃)₂ |
| A-1085 | CH₂(cyclo-C₃H₅) | CH₂—CH(CH₃)—CH₂—C(═CH₂)—CH₃ |
| A-1086 | CH₂(cyclo-C₃H₅) | C(═CH₂)—CH₂—CH₂—CH(CH₃)₂ |
| A-1087 | CH₂(cyclo-C₃H₅) | C(CH₃)═CH—CH₂—CH(CH₃)₂ |
| A-1088 | CH₂(cyclo-C₃H₅) | CH(CH₃)—CH═CH—CH(CH₃)₂ |
| A-1089 | CH₂(cyclo-C₃H₅) | CH(CH₃)—CH₂—CH═C(CH₃)₂ |
| A-1090 | CH₂(cyclo-C₃H₅) | CH(CH₃)—CH₂—CH₂—C(═CH₂)—CH₃ |
| A-1091 | CH₂(cyclo-C₃H₅) | CH═CH—C(CH₃)₂—CH₂—CH₃ |
| A-1092 | CH₂(cyclo-C₃H₅) | CH₂—CH₂—C(CH₃)₂—CH═CH₂ |
| A-1093 | CH₂(cyclo-C₃H₅) | CH═C(CH₃)—CH(CH₃)—CH₂—CH₃ |
| A-1094 | CH₂(cyclo-C₃H₅) | CH₂—C(═CH₂)—CH(CH₃)—CH₂—CH₃ |
| A-1095 | CH₂(cyclo-C₃H₅) | CH₂—C(CH₃)═C(CH₃)—CH₂—CH₃ |
| A-1096 | CH₂(cyclo-C₃H₅) | CH₂—CH(CH₃)—C(═CH₂)—CH₂—CH₃ |
| A-1097 | CH₂(cyclo-C₃H₅) | CH₂—CH(CH₃)—C(CH₃)═CH—CH₃ |
| A-1098 | CH₂(cyclo-C₃H₅) | CH₂—CH(CH₃)—CH(CH₃)—CH═CH₂ |
| A-1099 | CH₂(cyclo-C₃H₅) | C(═CH₂)—CH₂—CH(CH₃)—CH₂—CH₃ |
| A-1100 | CH₂(cyclo-C₃H₅) | C(CH₃)═CH—CH(CH₃)—CH₂—CH₃ |
| A-1101 | CH₂(cyclo-C₃H₅) | CH(CH₃)—CH═C(CH₃)—CH₂—CH₃ |
| A-1102 | CH₂(cyclo-C₃H₅) | CH(CH₃)—CH₂—C(═CH₂)—CH₂—CH₃ |
| A-1103 | CH₂(cyclo-C₃H₅) | CH(CH₃)—CH₂—C(CH₃)═CH—CH₃ |
| A-1104 | CH₂(cyclo-C₃H₅) | CH(CH₃)—CH₂—CH(CH₃)—CH═CH₂ |
| A-1105 | CH₂(cyclo-C₃H₅) | CH₂—C(CH₃)₂—CH═CH—CH₃ |
| A-1106 | CH₂(cyclo-C₃H₅) | CH₂—C(CH₃)₂—CH₂—CH═CH₂ |
| A-1107 | CH₂(cyclo-C₃H₅) | C(═CH₂)—CH(CH₃)—CH₂—CH₂—CH₃ |
| A-1108 | CH₂(cyclo-C₃H₅) | C(CH₃)═C(CH₃)—CH₂—CH₂—CH₃ |
| A-1109 | CH₂(cyclo-C₃H₅) | CH(CH₃)—C(═CH₂)—CH₂—CH₂—CH₃ |
| A-1110 | CH₂(cyclo-C₃H₅) | CH(CH₃)—C(CH₃)═CH—CH₂—CH₃ |
| A-1111 | CH₂(cyclo-C₃H₅) | CH(CH₃)—CH(CH₃)—CH═CH—CH₃ |
| A-1112 | CH₂(cyclo-C₃H₅) | CH(CH₃)—CH(CH₃)—CH₂—CH═CH₂ |
| A-1113 | CH₂(cyclo-C₃H₅) | C(CH₃)₂—CH═CH—CH₂—CH₃ |
| A-1114 | CH₂(cyclo-C₃H₅) | C(CH₃)₂—CH₂—CH═CH—CH₃ |
| A-1115 | CH₂(cyclo-C₃H₅) | C(CH₃)₂—CH₂—CH₂—CH═CH₂ |
| A-1116 | CH₂(cyclo-C₃H₅) | CH═CH—CH(CH₂—CH₃)—CH₂—CH₃ |
| A-1117 | CH₂(cyclo-C₃H₅) | CH₂—CH═C(CH₂—CH₃)—CH₂—CH₃ |
| A-1118 | CH₂(cyclo-C₃H₅) | CH₂—CH₂—C(═CH—CH₃)—CH₂—CH₃ |
| A-1119 | CH₂(cyclo-C₃H₅) | CH₂—CH₂—CH(CH═CH₂)—CH₂—CH₃ |
| A-1120 | CH₂(cyclo-C₃H₅) | CH═C(CH₂—CH₃)—CH₂—CH₂—CH₃ |
| A-1121 | CH₂(cyclo-C₃H₅) | CH₂—C(═CH—CH₃)—CH₂—CH₂—CH₃ |
| A-1122 | CH₂(cyclo-C₃H₅) | CH₂—CH(CH═CH₂)—CH₂—CH₂—CH₃ |
| A-1123 | CH₂(cyclo-C₃H₅) | CH₂—C(CH₂—CH₃)═CH—CH₂—CH₃ |
| A-1124 | CH₂(cyclo-C₃H₅) | CH₂—CH(CH₂—CH₃)—CH═CH—CH₃ |
| A-1125 | CH₂(cyclo-C₃H₅) | CH₂—CH(CH₂—CH₃)—CH—CH═CH₂ |
| A-1126 | CH₂(cyclo-C₃H₅) | C(═CH—CH₃)—CH₂—CH₂—CH₂—CH₃ |
| A-1127 | CH₂(cyclo-C₃H₅) | CH(CH═CH₂)—CH₂—CH₂—CH₂—CH₃ |
| A-1128 | CH₂(cyclo-C₃H₅) | C(CH₂—CH₃)═CH—CH₂—CH₂—CH₃ |
| A-1129 | CH₂(cyclo-C₃H₅) | CH(CH₂—CH₃)—CH═CH—CH₂—CH₃ |
| A-1130 | CH₂(cyclo-C₃H₅) | CH(CH₂—CH₃)—CH₂—CH═CH—CH₃ |
| A-1131 | CH₂(cyclo-C₃H₅) | CH(CH₂—CH₃)—CH₂—CH₂—CH═CH₂ |
| A-1132 | CH₂(cyclo-C₃H₅) | C(═CH—CH₂—CH₃)—CH₂—CH₂—CH₃ |
| A-1133 | CH₂(cyclo-C₃H₅) | C(CH═CH—CH₃)—CH₂—CH₂—CH₃ |
| A-1134 | CH₂(cyclo-C₃H₅) | C(CH₂—CH═CH₂)—CH₂—CH₂—CH₃ |
| A-1135 | CH₂(cyclo-C₃H₅) | CH═C(CH₃)—C(CH₃)₃ |
| A-1136 | CH₂(cyclo-C₃H₅) | CH₂—C(═CH₂)—C(CH₃)₃ |
| A-1137 | CH₂(cyclo-C₃H₅) | CH₂—C(CH₃)₂—CH(═CH₂)—CH₃ |
| A-1138 | CH₂(cyclo-C₃H₅) | C(═CH₂)—CH(CH₃)—CH(CH₃)—CH₃ |
| A-1139 | CH₂(cyclo-C₃H₅) | C(CH₃)═C(CH₃)—CH(CH₃)—CH₃ |
| A-1140 | CH₂(cyclo-C₃H₅) | CH(CH₃)—C(═CH₂)—CH(CH₃)—CH₃ |
| A-1141 | CH₂(cyclo-C₃H₅) | CH(CH₃)—C(CH₃)═C(CH₃)—CH₃ |
| A-1142 | CH₂(cyclo-C₃H₅) | CH(CH₃)—CH(CH₃)—C(═CH₂)—CH₃ |
| A-1143 | CH₂(cyclo-C₃H₅) | C(CH₃)₂—CH═C(CH₃)—CH₃ |
| A-1144 | CH₂(cyclo-C₃H₅) | C(CH₃)₂—CH₂—C(═CH₂)—CH₃ |
| A-1145 | CH₂(cyclo-C₃H₅) | C(CH₃)₂—C(═CH₂)—CH₂—CH₃ |
| A-1146 | CH₂(cyclo-C₃H₅) | C(CH₃)₂—C(CH₃)═CH—CH₃ |
| A-1147 | CH₂(cyclo-C₃H₅) | C(CH₃)₂—CH(CH₃)CH═CH₂ |
| A-1148 | CH₂(cyclo-C₃H₅) | CH(CH₂—CH₃)—CH₂—CH(CH₃)—CH₃ |
| A-1149 | CH₂(cyclo-C₃H₅) | CH(CH₂—CH₃)—CH(CH₃)—CH₂—CH₃ |
| A-1150 | CH₂(cyclo-C₃H₅) | C(CH₃)(CH₂—CH₃)—CH₂—CH₂—CH₃ |
| A-1151 | CH₂(cyclo-C₃H₅) | CH(i-C₃H₇)—CH₂—CH₂—CH₃ |
| A-1152 | CH₂(cyclo-C₃H₅) | CH═C(CH₂—CH₃)—CH(CH₃)—CH₃ |
| A-1153 | CH₂(cyclo-C₃H₅) | CH₂—C(═CH—CH₃)—CH(CH₃)—CH₃ |
| A-1154 | CH₂(cyclo-C₃H₅) | CH₂—CH(CH═CH₂)—CH(CH₃)—CH₃ |
| A-1155 | CH₂(cyclo-C₃H₅) | CH₂—C(CH₂—CH₃)═C(CH₃)—CH₃ |
| A-1156 | CH₂(cyclo-C₃H₅) | CH₂—CH(CH₂—CH₃)—(═CH₂)—CH₃ |
| A-1157 | CH₂(cyclo-C₃H₅) | CH₂—C(CH₃)(CH═CH₂)—CH₂—CH₃ |
| A-1158 | CH₂(cyclo-C₃H₅) | C(═CH₂)—CH(CH₂—CH₃)—CH₂—CH₃ |
| A-1159 | CH₂(cyclo-C₃H₅) | C(CH₃)═C(CH₂—CH₃)—CH₂—CH₃ |
| A-1160 | CH₂(cyclo-C₃H₅) | CH(CH₃)—C(═CH—CH₃)—CH₂—CH₃ |
| A-1161 | CH₂(cyclo-C₃H₅) | CH(CH₃)—CH(CH═CH₂)—CH₂—CH₃ |
| A-1162 | CH₂(cyclo-C₃H₅) | CH═C(CH₂—CH₃)—CH(CH₃)—CH₃ |
| A-1163 | CH₂(cyclo-C₃H₅) | CH₂—C(═CH—CH₃)—CH(CH₃)—CH₃ |
| A-1164 | CH₂(cyclo-C₃H₅) | CH₂—CH(CH═CH₂)—CH(CH₃)—CH₃ |
| A-1165 | CH₂(cyclo-C₃H₅) | CH₂—C(CH₂—CH₃)═C(CH₃)—CH₃ |
| A-1166 | CH₂(cyclo-C₃H₅) | CH₂—CH(CH₂—CH₃)—C(═CH₂)—CH₃ |
| A-1167 | CH₂(cyclo-C₃H₅) | C(═CH—CH₃)—CH₂—CH(CH₃)—CH₃ |
| A-1168 | CH₂(cyclo-C₃H₅) | CH(CH═CH₂)—CH₂—CH(CH₃)—CH₃ |
| A-1169 | CH₂(cyclo-C₃H₅) | C(CH₂—CH₃)═CH—CH(CH₃)—CH₃ |
| A-1170 | CH₂(cyclo-C₃H₅) | CH(CH₂—CH₃)CH═C(CH₃)—CH₃ |
| A-1171 | CH₂(cyclo-C₃H₅) | CH(CH₂—CH₃)CH₂—C(═CH₂)—CH₃ |
| A-1172 | CH₂(cyclo-C₃H₅) | C(═CH—CH₃)CH(CH₃)—CH₂—CH₃ |
| A-1173 | CH₂(cyclo-C₃H₅) | CH(CH═CH₂)CH(CH₃)—CH₂—CH₃ |
| A-1174 | CH₂(cyclo-C₃H₅) | C(CH₂—CH₃)═C(CH₃)—CH₂—CH₃ |
| A-1175 | CH₂(cyclo-C₃H₅) | CH(CH₂—CH₃)—C(═CH₂)—CH₂—CH₃ |
| A-1176 | CH₂(cyclo-C₃H₅) | CH(CH₂—CH₃)—C(CH₃)═CH—CH₃ |
| A-1177 | CH₂(cyclo-C₃H₅) | CH(CH₂—CH₃)—CH(CH₃)—CH═CH₂ |
| A-1178 | CH₂(cyclo-C₃H₅) | C(CH₃)(CH═CH₂)—CH₂—CH₂—CH₃ |
| A-1179 | CH₂(cyclo-C₃H₅) | C(CH₃)(CH₂—CH₃)—CH═CH—CH₃ |
| A-1180 | CH₂(cyclo-C₃H₅) | C(CH₃)(CH₂—CH₃)—CH₂—CH═CH₂ |
| A-1181 | CH₂(cyclo-C₃H₅) | C[═C(CH₃)—CH₃]—CH₂—CH₂—CH₃ |
| A-1182 | CH₂(cyclo-C₃H₅) | CH[C(═CH₂)—CH₃]—CH₂—CH₂—CH₃ |
| A-1183 | CH₂(cyclo-C₃H₅) | C(i-C₃H₇)═CH—CH₂—CH₃ |
| A-1184 | CH₂(cyclo-C₃H₅) | CH(i-C₃H₇)—CH═CH—CH₃ |
| A-1185 | CH₂(cyclo-C₃H₅) | CH(i-C₃H₇)—CH₂—CH═CH₂ |
| A-1186 | CH₂(cyclo-C₃H₅) | C(═CH—CH₃)—C(CH₃)₃ |
| A-1187 | CH₂(cyclo-C₃H₅) | CH(CH═CH₂)—C(CH₃)₃ |
| A-1188 | CH₂(cyclo-C₃H₅) | C(CH₃)(CH═CH₂)CH(CH₃)—CH₃ |
| A-1189 | CH₂(cyclo-C₃H₅) | C(CH₃)(CH₂—CH₃)C(═CH₂)—CH₃ |
| A-1190 | CH₂(cyclo-C₃H₅) | 2-CH₃-cyclohex-1-enyl |
| A-1191 | CH₂(cyclo-C₃H₅) | [2-(═CH₂)]-cyclo-C₆H₉ |
| A-1192 | CH₂(cyclo-C₃H₅) | 2-CH₃-cyclohex-2-enyl |
| A-1193 | CH₂(cyclo-C₃H₅) | 2-CH₃-cyclohex-3-enyl |
| A-1194 | CH₂(cyclo-C₃H₅) | 2-CH₃-cyclohex-4-enyl |
| A-1195 | CH₂(cyclo-C₃H₅) | 2-CH₃-cyclohex-5-enyl |
| A-1196 | CH₂(cyclo-C₃H₅) | 2-CH₃-cyclohex-6-enyl |
| A-1197 | CH₂(cyclo-C₃H₅) | 3-CH₃-cyclohex-1-enyl |
| A-1198 | CH₂(cyclo-C₃H₅) | 3-CH₃-cyclohex-2-enyl |
| A-1199 | CH₂(cyclo-C₃H₅) | [3-(═CH₂)]-cyclo-C₆H₉ |
| A-1200 | CH₂(cyclo-C₃H₅) | 3-CH₃-cyclohex-3-enyl |
| A-1201 | CH₂(cyclo-C₃H₅) | 3-CH₃-cyclohex-4-enyl |
| A-1202 | CH₂(cyclo-C₃H₅) | 3-CH₃-cyclohex-5-enyl |
| A-1203 | CH₂(cyclo-C₃H₅) | 3-CH₃-cyclohex-6-enyl |
| A-1204 | CH₂(cyclo-C₃H₅) | 4-CH₃-cyclohex-1-enyl |
| A-1205 | CH₂(cyclo-C₃H₅) | 4-CH₃-cyclohex-2-enyl |
| A-1206 | CH₂(cyclo-C₃H₅) | 4-CH₃-cyclohex-3-enyl |
| A-1207 | CH₂(cyclo-C₃H₅) | [4-(═CH₂)]-cyclo-C₆H₉ |
| A-1208 | CH₂(cyclo-C₃H₅) | CH₂CF₃ |
| A-1209 | CH₂(cyclo-C₃H₅) | CH₂CCl₃ |
| A-1210 | CH₂(cyclo-C₃H₅) | CH₂(cyclo-C₃H₅) |

| | |
|---|---|
| A-1211 | —CH₂—CH₂—CH₂—CH₂— |
| A-1212 | —CH₂—CH₂—CH₂— |
| A-1213 | —CH₂—CH₂—CH(CH₃)— |
| A-1214 | —CH(CH₃)—CH₂—CH₂— |
| A-1215 | —CH₂—C(CH₃)₂—CH₂— |
| A-1216 | —CH(CH₃)—CH₂—CH₂—CH₂— |
| A-1217 | —CH₂—CH₂—CH₂—CH(CH₃)— |
| A-1218 | —CH₂—CH₂—CH(CH₃)—CH₂— |
| A-1219 | —CH₂—CH(CH₃)—CH₂—CH₂— |
| A-1220 | —C(CH₃)₂—CH₂—CH₂—CH₂— |
| A-1221 | —C(CH₃)₂—CH₂—CH₂—CH₂— |
| A-1222 | —CH₂—CH₂—CH₂—C(CH₃)₂— |
| A-1223 | —CH₂—C(CH₃)₂—CH₂—CH₂— |
| A-1224 | —CH₂—CH₂—C(CH₃)₂—CH₂— |
| A-1225 | (RR)—CH(CH₃)—CH(CH₃)— |
| A-1226 | (SS)—CH(CH₃)—CH(CH₃)— |
| A-1227 | (RS)—CH(CH₃)—CH(CH₃)— |
| A-1228 | (SR)—CH(CH₃)—CH(CH₃)— |
| A-1229 | —C(CH₃)₂—CH₂—CH₂— |
| A-1230 | —CH(C₂H₅)—CH₂—CH₂—CH₂— |
| A-1231 | —CH₂—CH₂—CH₂—CH(C₂H₅₎— |
| A-1232 | —CH₂—CH₂—CH(C₂H₅)—CH₂— |
| A-1233 | —CH₂—CH(C₂H₅)—CH₂—CH₂— |
| A-1234 | —CH₂—CH₂—C(CH₃)₂— |

The compounds according to the invention and/or their agriculturally acceptable salts are suitable as fungicides. They are distinguished by excellent activity against a broad spectrum of phytopathogenic fungi from the class of the Ascomycetes, Deuteromycetes, Basidiomycetes and Peronosporomycetes (syn. Oomycetes). Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetables, such as cucumbers, beans, tomatoes, potatoes and cucurbits, and also on the seeds of these plants.

The compounds I are suitable for controlling Alternaria species on vegetables, rapeseed, sugarbeet and fruit and rice, such as, for example, A. solani or A. alternata on potatoes and tomatoes.

The compounds I are suitable for controlling Aphanomyces species on sugarbeet and vegetables.

The compounds I are suitable for controlling Ascochyta species on cereals and vegetables.

The compounds I are suitable for controlling Bipolaris and Drechslera species on corn, cereals, rice and lawns, such as, for example, D. maydis on corn.

The compounds I are suitable for controlling Blumeria graminis (powdery mildew) on cereals.

The compounds I are suitable for controlling Botrytis cinerea (gray mold) on strawberries, vegetables, flowers and grapevines.

The compounds I are suitable for controlling Bremia lactucae on lettuce.

The compounds I are suitable for controlling Cercospora species on corn, soybeans, rice and sugarbeet.

The compounds I are suitable for controlling Cochliobolus species on corn, cereals, rice, such as, for example, Cochliobolus sativus on cereals, Cochliobolus miyabeanus on rice.

The compounds I are suitable for controlling Colletotricum species on soybeans and cotton.

The compounds I are suitable for controlling Drechslera species, Pyrenophora species on corn, cereals, rice and lawns, such as, for example, D. teres on barley or D. tritici-repentis on wheat.

The compounds I are suitable for controlling Esca on grapevines, caused by Phaeoacremonium chlamydosporium, Ph. Aleophilum, and Formitipora punctata (syn. Phellinus punctatus).

The compounds I are suitable for controlling Elisinoe ampelina on grapevines.

The compounds I are suitable for controlling Exserohilum species on corn.

The compounds I are suitable for controlling Erysiphe cichoracearum and Sphaerotheca fuliginea on cucumbers.

The compounds I are suitable for controlling Fusarium and Verticillium species on various plants, such as, for example, F. graminearum or F. culmorum on cereals or F. oxysporum on a large number of plants, such as, for example, tomatoes.

The compounds I are suitable for controlling Gaeumanomyces graminis on cereals.

The compounds I are suitable for controlling Gibberella species on cereals and rice (for example Gibberella fujikuroi on rice).

The compounds I are suitable for controlling Glomerella cingulata on grapevines and other plants.

The compounds I are suitable for controlling Grainstaining complex on rice.

The compounds I are suitable for controlling Guignardia budwelli on grapevines.

The compounds I are suitable for controlling Helminthosporium species on corn and rice.

The compounds I are suitable for controlling Isariopsis clavispora on grapevines.

The compounds I are suitable for controlling Michrodochium nivale on cereals.

The compounds I are suitable for controlling Mycosphaerella species on cereals, bananas and peanuts, such as, for example, M. graminicola on wheat or M. fijiensis on bananas.

The compounds I are suitable for controlling Peronospora species on cabbage and bulbous plants, such as, for example, P. brassicae on cabbage or P. destructor on onions.

The compounds I are suitable for controlling Phakopsara pachyrhizi and Phakopsara meibomiae on soybeans.

The compounds I are suitable for controlling Phomopsis species on soybeans and sunflowers.

The compounds I are suitable for controlling Phytophthora infestans on potatoes and tomatoes.

The compounds I are suitable for controlling Phytophthora species on various plants, such as, for example, P. capsici on bell peppers.

The compounds I are suitable for controlling Plasmopara viticola on grapevines.

The compounds I are suitable for controlling Podosphaera leucotricha on apples.

The compounds I are suitable for controlling Pseudocercosporella herpotrichoides on cereals.

The compounds I are suitable for controlling Pseudoperonospora on various plants, such as, for example, P. cubensis on cucumbers or P. humili on hops.

The compounds I are suitable for controlling Pseudopezicula tracheiphilai on grapevines.

The compounds I are suitable for controlling Puccinia species on various plants, such as, for example, P. triticina, P. striformins, P. hordei or P. graminis on cereals, or P. asparagi on asparagus.

The compounds I are suitable for controlling Pyricularia oryzae, Corticium sasakii, Sarocladium oryzae, S. attenuatum, Entyloma oryzae on rice.

The compounds I are suitable for controlling Pyricularia grisea on lawns and cereals. The compounds I are suitable for controlling Pythium spp. on lawns, rice, corn, cotton, rapeseed, sunflowers, sugarbeet, vegetables and other plants, such as, for example, P. ultiumum on various plants, P. aphanidermatum on lawns.

The compounds I are suitable for controlling Rhizoctonia species on cotton, rice, potatoes, lawns, corn, rapeseed, potatoes, sugarbeet, vegetables and on various plants, such as, for example, R. solani on beet and various plants.

The compounds I are suitable for controlling Rhynchosporium secalis on barley, rye and triticale.

The compounds I are suitable for controlling Sclerotinia species on rapeseed and sunflowers.

The compounds I are suitable for controlling Septoria tritici and Stagonospora nodorum on wheat.

The compounds I are suitable for controlling Erysiphe (syn. Uncinula) necator on grapevines.

The compounds I are suitable for controlling Setospaeria species on corn and lawns.

The compounds I are suitable for controlling Sphacelotheca reilinia on corn.

The compounds I are suitable for controlling Thievaliopsis species on soybeans and cotton.

The compounds I are suitable for controlling Tilletia species on cereals.

The compounds I are suitable for controlling Ustilago species on cereals, corn and sugarcane, such as, for example, U. maydis on corn.

The compounds I are suitable for controlling Venturia species (scab) on apples and pears, such as, for example, V. inaequalis on apples.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

Accordingly, the present invention furthermore provides the use of the compounds according to the invention and/or their agriculturally acceptable salts for controlling phytopathogenic fungi.

In addition, the compounds according to the invention can also be used in crops which, owing to breeding including genetical engineering, are tolerant to attack by insects or fungi.

The compounds according to the invention and/or their agriculturally acceptable salts are employed by treating the fungi active or the plants, seeds, materials or soil to be protected from fungal attack with a fungicidally effective amount of these compounds. The application can be carried out both before and after the infection of the materials, plants or seeds by the fungi.

Accordingly, the present invention furthermore provides a method for controlling phytopathogenic fungi, which method comprises treating the fungi or the materials, plants, the soil or seed to be protected against fungal attack with an effective amount of at least one compound according to the invention and/or an agriculturally acceptable salt thereof.

The present invention furthermore provides a composition for controlling phytopathogenic fungi, which composition comprises at least one compound according to the invention and/or an agriculturally acceptable salt thereof and at least one solid or liquid carrier.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In the treatment of seed, the amounts of active compound employed are generally from 1 to 1000 g/100 kg, preferably from 1 to 200 g/100 kg, in particular from 5 to 100 g/100 kg of seed.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds according to the invention and/or their agriculturally acceptable salts can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Products to be Applied Undiluted

I Dustable powders (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

K ULV solutions (UL)

10 parts by weight of the active compounds are dissolved in 90 parts by weight of an organic solvent, for example xylene. This gives a product with an active compound content of 10% by weight to be applied undiluted.

Seed treatment typically utilizes water-soluble concentrates (LS), suspensions (FS), dusts (DS), water-dispersible and water-soluble powders (WS, SS), emulsions (ES), emulsifiable concentrates (EC) and gel formulations (GF). These formulations can be applied neat or preferably diluted to the seed. The application can take place prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compounds according to the invention can, in the application form as fungicides, also be present together with other active compounds, for example with herbicides, insecticides, growth regulators, fungicides or also with fertilizers. When mixing the compounds according to the invention or the compositions comprising them with one or more further active compounds, in particular fungicides, it is in many cases possible for example to broaden the activity spectrum or to prevent the development of resistance. In many cases, synergistic effects are obtained.

The invention furthermore provides therefore a composition of at least one compound according to the invention and/or an agriculturally acceptable salt thereof and at least one further fungicidally, insecticidally, herbicidally and/or growth-regulating active compound.

The present invention also provides a pesticidal composition comprising at least one compound I, in particular a compound I described in the present description as being preferred and/or an agriculturally acceptable salt thereof, and at least one solid or liquid carrier. Such a pesticidal composition may comprise at least one further fungicidally, insecticidally and/or herbicidally active compound.

The following list L of fungicides, together with which the compounds according to the invention may be used, is meant to illustrate the combination possibilities, but not to limit them:

### List L:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-((2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluoro-biphenyl-2-yl)-3-difluoromethyl-1-methylpyrazole-4-carboxamide, N-(3′,4′-dichloro-5-fluoro-biphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds:

pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol,
pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometal compounds: fentin salts;
sulfur-containing heterocyclyl compounds: isoprothiolane, dithianon;
organophosphorous compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

Accordingly, the present invention furthermore relates to the compositions listed in Table B, where a row of Table B corresponds in each case to a fungicidal composition comprising a compound of the formula I (component 1), which is preferably one of the compounds described herein as being preferred, and the respective further active compound (component 2) stated in the row in question. According to one embodiment of the invention, component 1 in each row of Table B is in each case one of the compounds of the formula I specifically individualized in Tables 1 to 184.

TABLE B

| No. | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | picoxystrobin |
| B-8 | a compound of the formula I | pyraclostrobin |
| B-9 | a compound of the formula I | trifloxystrobin |
| B-10 | a compound of the formula I | orysastrobin |
| B-11 | a compound of the formula I | methyl (2-chloro-5-[1-(3-methylbenzyloxyimino) |
| | | ethyl]benzyl)carbamate |
| B-12 | a compound of the formula I | methyl (2-chloro-5-[1-(6-methylpyridin-2-yl- |
| | | methoxyimino)ethyl]benzyl)carbamate |
| B-13 | a compound of the formula I | methyl 2-(ortho-(2,5-dimethylphenyloxy- |
| | | methylene)phenyl)-3-methoxyacrylate |
| B-14 | a compound of the formula I | benalaxyl |
| B-15 | a compound of the formula I | benodanil |
| B-16 | a compound of the formula I | boscalid |
| B-17 | a compound of the formula I | carboxin |
| B-18 | a compound of the formula I | mepronil |
| B-19 | a compound of the formula I | fenfuram |
| B-20 | a compound of the formula I | fenhexamid |
| B-21 | a compound of the formula I | flutolanil |
| B-22 | a compound of the formula I | furametpyr |
| B-23 | a compound of the formula I | metalaxyl |
| B-24 | a compound of the formula I | ofurace |
| B-25 | a compound of the formula I | oxadixyl |
| B-26 | a compound of the formula I | oxycarboxin |
| B-27 | a compound of the formula I | penthiopyrad |
| B-28 | a compound of the formula I | thifluzamide |
| B-29 | a compound of the formula I | tiadinil |
| B-30 | a compound of the formula I | N-(4′-bromobiphenyl-2-yl)-4-difluoromethyl-2- |
| | | methylthiazole-5-carboxamide |
| B-31 | a compound of the formula I | N-(4′-trifluoromethylbiphenyl-2-yl)-4-di- |
| | | fluoromethyl-2-methylthiazole-5-carboxamide |
| B-32 | a compound of the formula I | N-(4′-chloro-3′-fluorobiphenyl-2-yl)-4-di- |
| | | fluoromethyl-2-methylthiazole-5-carboxamide |
| B-33 | a compound of the formula I | N-(3′,4′-dichloro-4-fluorobiphenyl-2-yl)-3-di- |
| | | fluoromethyl-1-methylpyrazole-4-carboxamide |
| B-34 | a compound of the formula I | N-(3′,4′-dichloro-5-fluorobiphenyl-2-yl)-3-di- |
| | | fluoromethyl-1-methylpyrazole-4-carboxamide |
| B-35 | a compound of the formula I | N-(2-cyanophenyl)-3,4-dichloroisothiazole-5- |
| | | carboxamide |
| B-36 | a compound of the formula I | dimethomorph |
| B-37 | a compound of the formula I | flumorph |
| B-38 | a compound of the formula I | flumetover |
| B-39 | a compound of the formula I | fluopicolide (picobenzamid) |
| B-40 | a compound of the formula I | zoxamide |
| B-41 | a compound of the formula I | carpropamid |
| B-42 | a compound of the formula I | diclocymet |
| B-43 | a compound of the formula I | mandipropamid |
| B-44 | a compound of the formula I | N-(2-(4-[3-(4-chlorophenyl)prop-2-ynyloxy]-3- |
| | | methoxyphenyl)ethyl)-2-methanesulfonyl- |
| | | amino-3-methylbutyramide |
| B-45 | a compound of the formula I | N-(2-(4-[3-(4-chlorophenyl)prop-2-ynyloxy]-3- |
| | | methoxyphenyl)ethyl)-2-ethanesulfonylamino- |
| | | 3-methylbutyramide |
| B-46 | a compound of the formula I | bitertanol |
| B-47 | a compound of the formula I | bromuconazole |
| B-48 | a compound of the formula I | cyproconazole |
| B-49 | a compound of the formula I | difenoconazole |
| B-50 | a compound of the formula I | diniconazole |
| B-51 | a compound of the formula I | enilconazole |
| B-52 | a compound of the formula I | epoxiconazole |
| B-53 | a compound of the formula I | fenbuconazole |
| B-54 | a compound of the formula I | flusilazole |
| B-55 | a compound of the formula I | fluquinconazole |
| B-56 | a compound of the formula I | flutriafol |
| B-57 | a compound of the formula I | hexaconazol |
| B-58 | a compound of the formula I | imibenconazole |
| B-59 | a compound of the formula I | ipconazole |
| B-60 | a compound of the formula I | metconazol |
| B-61 | a compound of the formula I | myclobutanil |
| B-62 | a compound of the formula I | penconazole |
| B-63 | a compound of the formula I | propiconazole |
| B-64 | a compound of the formula I | prothioconazole |
| B-65 | a compound of the formula I | simeconazole |
| B-66 | a compound of the formula I | tebuconazole |
| B-67 | a compound of the formula I | tetraconazole |
| B-68 | a compound of the formula I | triadimenol |
| B-69 | a compound of the formula I | triadimefon |
| B-70 | a compound of the formula I | triticonazole |
| B-71 | a compound of the formula I | cyazofamid |
| B-72 | a compound of the formula I | imazalil |
| B-73 | a compound of the formula I | pefurazoate |
| B-74 | a compound of the formula I | prochloraz |
| B-75 | a compound of the formula I | triflumizole |
| B-76 | a compound of the formula I | benomyl |
| B-77 | a compound of the formula I | carbendazim |
| B-78 | a compound of the formula I | fuberidazole |
| B-79 | a compound of the formula I | thiabendazole |
| B-80 | a compound of the formula I | ethaboxam |
| B-81 | a compound of the formula I | etridiazole |
| B-82 | a compound of the formula I | hymexazole |
| B-83 | a compound of the formula I | fluazinam |
| B-84 | a compound of the formula I | pyrifenox |
| B-85 | a compound of the formula I | 3-[5-(4-chlorophenyl)-2,3-dimethylisoxazolidin- |
| | | 3-yl]pyridine |
| B-86 | a compound of the formula I | bupirimate |
| B-87 | a compound of the formula I | cyprodinil |
| B-88 | a compound of the formula I | ferimzone |
| B-89 | a compound of the formula I | fenarimol |
| B-90 | a compound of the formula I | mepanipyrim |
| B-91 | a compound of the formula I | nuarimol |
| B-92 | a compound of the formula I | pyrimethanil |
| B-93 | a compound of the formula I | triforine |
| B-94 | a compound of the formula I | fludioxonil |
| B-95 | a compound of the formula I | fenpiclonil |
| B-96 | a compound of the formula I | aldimorph |
| B-97 | a compound of the formula I | dodemorph |
| B-98 | a compound of the formula I | fenpropimorph |
| B-99 | a compound of the formula I | tridemorph |
| B-100 | a compound of the formula I | iprodione |
| B-101 | a compound of the formula I | procymidone |
| B-102 | a compound of the formula I | vinclozolin |
| B-103 | a compound of the formula I | acibenzolar-S-methyl |
| B-104 | a compound of the formula I | anilazin |
| B-105 | a compound of the formula I | captan |
| B-106 | a compound of the formula I | captafol |
| B-107 | a compound of the formula I | dazomet |
| B-108 | a compound of the formula I | diclomezine |
| B-109 | a compound of the formula I | fenoxanil |
| B-110 | a compound of the formula I | folpet |
| B-111 | a compound of the formula I | fenpropidin |
| B-112 | a compound of the formula I | famoxadone |
| B-113 | a compound of the formula I | fenamidone |
| B-114 | a compound of the formula I | octhilinone |
| B-115 | a compound of the formula I | probenazole |
| B-116 | a compound of the formula I | proquinazid |
| B-117 | a compound of the formula I | pyroquilon |
| B-118 | a compound of the formula I | quinoxyfen |
| B-119 | a compound of the formula I | tricyclazole |
| B-120 | a compound of the formula I | 5-chloro-7-(4-methylpiperidin-1-yl)-6-(2,4,6- |
| | | trifluorophenyl)-[1,2,4]triazolo[1,5-a]pyrimidine |
| B-121 | a compound of the formula I | 2-butoxy-6-iodo-3-propylchromene-4-one |
| B-122 | a compound of the formula I | N,N-dimethyl-3-(3-bromo-6-fluoro-2-methyl- |
| | | indole-1-sulfonyl)-[1,2,4]triazole-1-sulfonamide |
| B-123 | a compound of the formula I | ferbam |
| B-124 | a compound of the formula I | mancozeb |
| B-125 | a compound of the formula I | maneb |
| B-126 | a compound of the formula I | metiram |
| B-127 | a compound of the formula I | metam |
| B-128 | a compound of the formula I | propineb |
| B-129 | a compound of the formula I | thiram |
| B-130 | a compound of the formula I | zineb |
| B-131 | a compound of the formula I | ziram |
| B-132 | a compound of the formula I | diethofencarb |
| B-133 | a compound of the formula I | flubenthiavalicarb |
| B-134 | a compound of the formula I | iprovalicarb |
| B-135 | a compound of the formula I | propamocarb |
| B-136 | a compound of the formula I | methyl 3-(4-chlorophenyl)-3-(2-isopropoxy- |
| | | carbonylamino-3-methylbutyrylamino)- |
| | | propionate |
| B-137 | a compound of the formula I | 4-fluorophenyl N-(1-(1-(4-cyanophenyl)- |
| | | ethanesulfonyl)but-2-yl)carbamate |
| B-138 | a compound of the formula I | dodine |
| B-139 | a compound of the formula I | iminoctadine |
| B-140 | a compound of the formula I | guazatine |
| B-141 | a compound of the formula I | kasugamycin |
| B-142 | a compound of the formula I | polyoxine |
| B-143 | a compound of the formula I | streptomycin |
| B-144 | a compound of the formula I | validamycin A |
| B-145 | a compound of the formula I | fentin salts |
| B-146 | a compound of the formula I | isoprothiolane |
| B-147 | a compound of the formula I | dithianon |
| B-148 | a compound of the formula I | edifenphos |
| B-149 | a compound of the formula I | fosetyl |
| B-150 | a compound of the formula I | fosetyl-aluminum |
| B-151 | a compound of the formula I | iprobenfos |
| B-152 | a compound of the formula I | pyrazophos |
| B-153 | a compound of the formula I | tolclofos-methyl |
| B-154 | a compound of the formula I | phosphorous acid and its salts |
| B-155 | a compound of the formula I | thiophanate methyl |
| B-156 | a compound of the formula I | chlorothalonil |
| B-157 | a compound of the formula I | dichlofluanid |
| B-158 | a compound of the formula I | tolylfluanid |
| B-159 | a compound of the formula I | flusulfamide |
| B-160 | a compound of the formula I | phthalide |
| B-161 | a compound of the formula I | hexachlorobenzene |
| B-162 | a compound of the formula I | pencycuron |
| B-163 | a compound of the formula I | quintozene |
| B-164 | a compound of the formula I | binapacryl |
| B-165 | a compound of the formula I | dinocap |
| B-166 | a compound of the formula I | dinobuton |
| B-167 | a compound of the formula I | Bordeaux mixture |
| B-168 | a compound of the formula I | copper acetate |
| B-169 | a compound of the formula I | copper hydroxide |
| B-170 | a compound of the formula I | copper oxychloride |
| B-171 | a compound of the formula I | basic copper sulfate |
| B-172 | a compound of the formula I | sulfur |
| B-173 | a compound of the formula I | spiroxamine |
| B-174 | a compound of the formula I | cyflufenamid |
| B-175 | a compound of the formula I | cymoxanil |
| B-176 | a compound of the formula I | metrafenone |

The active compounds II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known [cf. in EP-A 226 917; EP-A 10 28 125; EP-A 10 35 122; EP-A 12 01 648; WO 98/46608; WO 99/24413; WO 03/14103; WO 03/053145; WO 03/066609 and WO 04/049804].

The present invention furthermore relates to the pharmaceutical use of the compounds according to the invention, in particular the compounds according to the invention described as preferred, and/or the pharmaceutically acceptable salts thereof, in particular their use for controlling tumors in mammals such as, for example, humans.

The compounds according to the invention and/or their agriculturally acceptable salts can be present in various crystal modifications which may differ in their biological activity. They are also provided by the present invention.

### SYNTHESIS EXAMPLES

With appropriate modification of the starting materials, the procedures given in the synthesis examples below were used to obtain further compounds according to the invention:

### Example A

### 4-Chloro-6-(6-methyltetrahydro-2H-(1,2)-oxazin-2-yl)-2-(1,2,4-triazol-1-yl)-5-(2,4,6-trifluorophenyl)pyrimidine (Table C, No. 21)

### Aa) 2-Ethoxycarbonyl-6-methyltetrahydro-2H-(1,2)-oxazine

12.23 g (185.3 mmol) of potassium hydroxide were dissolved in 100 ml of ethanol, and, at room temperature, 19.47 g (185.3 mmol) of O-ethyl N-hydroxyurethane and then 21.29 g (92.6 mmol) of 1,4-dibromopentane were added a little at a time with stirring. The mixture was boiled under reflux for 6 h and concentrated under reduced pressure, and the residue was suspended in 300 ml of methyl tert-butyl ether and filtered. The filtrate was again concentrated under reduced pressure. The residue (13.27 g) was then directly reacted further.

### Ab) 6-Methyltetrahydro-2H-(1,2)-oxazine

The product from the preceding experiment was boiled in 300 g of 10% strength hydrochloric acid for 2 h. The solution was washed three times with in each case 100 ml of methyl tert-butyl ether and concentrated under reduced pressure. The residue was dissolved in warm diisopropyl ether, filtered and again concentrated under reduced pressure. 10 ml of 50% strength aqueous sodium hydroxide solution were added to the residue (8.1 g) and the mixture was subjected to fractional distillation at atmospheric pressure. The product was collected at a transition temperature of 85-95° C. The product (4.96 g) was directly used further.

### Ac) 4-Chloro-6-(6-methyltetrahydro-2H-(1,2)-oxazin-2-yl)-2-methylthio-5-(2,4,6-trifluorophenyl)pyrimidine

At room temperature, 2.5 g of 6-methyltetrahydro-2H-(1,2)-oxazine from the preceding experiment, 4.2 g (12.9 mmol) of 4,6-dichloro-2-methylthio-5-(2,4,6-trifluorophenyl)pyrimidine and 2.2 g (21.5 mmol) of triethylamine were dissolved in 20 ml of dimethyl sulfoxide and then stirred at 100° C. for 5 h. The mixture was then added to 200 ml of water and 100 ml of methyl tert-butyl ether, the pH was adjusted to 8 using sodium bicarbonate and the organic phase was separated off. The aqueous phase was extracted twice with in each case 100 ml of methyl tert-butyl ether and the combined organic phases were dried over sodium sulfate and concentrated under reduced pressure. The crude product (5.1 g) was purified by chromatography on silica gel using cyclohexane/methyl tert-butyl ether. Yield 4.47 g, m.p. 110-111° C.

### Ad) 4-Chloro-2-methylsulfonyl-6-(6-methyltetrahydro-2H-(1,2)-oxazin-2-yl)-5-(2,4,6-trifluorophenyl)pyrimidine

4.40 g (11.3 mmol) of 4-chloro-6-(6-methyltetrahydro-2H-(1,2)-oxazin-2-yl)-2-methylthio-5-(2,4,6-trifluorophenyl)pyrimidine were dissolved in 50 ml of methylene chloride, and, at 0-5° C., 5.56 g of 3-chloroperbenzoic acid were added a little at a time with stirring. At 5° C., the mixture was stirred for 7 h, another 0.5 g of 3-chloroperbenzoic acid was added, the mixture was stirred at 0° C. for 5 h and concentrated under reduced pressure and the residue was dissolved in 30 ml of ethyl acetate, washed three times with in each case 15 ml of saturated sodium bicarbonate solution, dried over sodium sulfate, concentrated under reduced pressure, triturated with diisopropyl ether and dried under reduced pressure. Yield 2.9 g, m.p. 160-162° C.

### Ae) 4-Chloro-6-(6-methyltetrahydro-2H-(1,2)-oxazin-2-yl)-2-(1,2,4-triazol-1-yl)-5-(2,4,6-trifluorophenyl)pyrimidine

At room temperature, 26 mg (1.04 mmol) of 95% pure sodium hydride were initially charged in 4.0 ml of tetrahydrofuran, 69 mg (1.00 mmol) of (1,2,4)-triazole were then added and the mixture was stirred for 3 h. 0.40 g (0.95 mmol) of 4-chloro-2-methylsulfonyl-6-(6-methyltetrahydro-2H-(1,2)-oxazin-2-yl)-5-(2,4,6-trifluorophenyl)pyrimidine was then added and the mixture was stirred at room temperature overnight. After addition of 20 ml of methyl tert-butyl ether, the mixture was washed three times with in each case 3 ml of water, dried over sodium sulfate, concentrated under reduced pressure and purified by chromatography on silica gel using cyclohexane/ethyl acetate. Yield 140 mg, m.p. 141-143° C. ¹H-NMR (CDCl₃) δ=1.37 (d); 1.45-2.10 (m); 3.50 (m); 5.13 (m); 6.75 (m); 8.15 (s); 9.15 (s).

### Example B

### N-Methoxy-4-chloro-6-(isoxazolidin-2-yl)-5-(2,4,6-trifluorophenyl)-2-pyrimidinecarboximidamide (Table C, No. 17)

### Ba) 4-Chloro-6 (isoxazolidin-2-yl)-2-methylthio-5-(2,4,6-trifluorophenyl)pyrimidine

At room temperature, 2.02 g (18.5 mmol) of isoxazolidine hydrochloride, 5.00 g (15.4 mmol) of 4,6-dichloro-2-methylthio-5-(2,4,6-trifluorophenyl)pyrimidine and 3.42 g (33.8 mmol) of triethylamine were dissolved in 20 ml of dimethyl sulfoxide and then stirred at 10° C. for 5 h. The mixture was added to 200 ml of water and 100 ml of methyl tert-butyl ether, the pH was adjusted to 8 using sodium bicarbonate and the organic phase was separated off. The aqueous phase was extracted twice with in each case 100 ml of methyl tert-butyl ether and the combined organic phases were dried over sodium sulfate and concentrated under reduced pressure. The crude product was purified by chromatography on silica gel using cyclohexane/methyl tert-butyl ether. Yield 2.6 g, m.p. 106-109° C.

### Bb) 4-Chloro-6-(isoxazolidin-2-yl)-2-methylsulfonyl-5-(2,4,6-trifluorophenyl)pyrimidine

2.60 g (7.19 mmol) of 4-chloro-6-(isoxazolidin-2-yl)-2-methylthio-5-(2,4,6-trifluorophenyl)pyrimidine were dissolved in 30 ml of methylene chloride, and at 0° C. 3.54 g of 3-chloroperbenzoic acid were added a little at a time, with stirring. The mixture was stirred at 0° C. for 6 h and then at room temperature for 3 d. After the addition of 300 ml of ethyl acetate, the mixture was washed three times with in each case 100 ml of saturated sodium bicarbonate solution, dried over sodium sulfate and concentrated under reduced pressure. The crude product (3.6 g) was purified by chromatography on silica gel using cyclohexane/methyl tert-butyl ether/ethyl acetate. Yield 2.0 g, m.p. 210-219° C.

### Bc) 4-Chloro-2-cyano-6-(isoxazolidin-2-yl)-5-(2,4,6-trifluorophenyl)pyrimidine

At room temperature, 1.50 g (3.81 mmol) of 4-chloro-6-(isoxazolidin-2-yl)-2-methylsulfonyl-5-(2,4,6-trifluorophenyl)pyrimidine, 397 mg (6.09 mmol) of potassium cyanide and 13 mg of crown ether (18-crown-6) were added to 15 ml of acetonitrile. The mixture was stirred for 1 d and concentrated under reduced pressure, 50 ml of ethyl acetate were added and the mixture was washed three times with in each case 20 ml of water, dried over sodium sulfate and concentrated under reduced pressure. The crude product (1.7 g) was purified by chromatography on silica gel using cyclohexane/methyl tert-butyl ether. Yield 0.68 g, m.p. 107-113° C.

### Bd) N-Methoxy-4-chloro-6-(isoxazolidin-2-yl)-5-(2,4,6-trifluorophenyl)-2-pyrimidine-carboximidamide

At 0° C., 400 mg (1.17 mmol) of 4-chloro-2-cyano-6-(isoxazolidin-2-yl)-5-(2,4,6-trifluorophenyl)pyrimidine and 127 mg (1.17 mmol) of 50% strength sodium methoxide solution in methanol were stirred in 5 ml of methanol for 1 d. 118 mg (1.41 mmol) of methoxyamine hydrochloride were then added, the mixture was stirred at room temperature for 6 h and concentrated under reduced pressure, 12 ml of ethyl acetate and 12 ml of saturated sodium bicarbonate solution were added, the aqueous phase was separated off and extracted with 12 ml of ethyl acetate and the combined organic phases were dried over sodium sulfate and concentrated under reduced pressure. Recrystallisation from acetonitrile gave 180 mg, m.p. 192-193° C. ¹H-NMR (CDCl₃) δ=2.25 (m); 3.73 (m); 4.00 (m); 4.05 (s); 5.41 (br); 6.74 (m).

### Example C

### 4-Chloro-6-(isoxazolidin-2-yl)-5-(2,4,6-trifluorophenyl)-2-pyrimidinecarboxmide (Table C, No. 19)

22 mg (0.16 mmol) of potassium carbonate and 94 mg (0.83 mmol) of 30% strength hydrogen peroxide were added to 270 mg (0.79 mmol) of 4-chloro-2-cyano-6-(isoxazolidin-2-yl)-5-(2,4,6-trifluorophenyl)pyrimidine in 1 ml of dimethyl sulfoxide, and the mixture was stirred at room temperature for 16 h. The mixture was added to 15 ml of water and extracted three times with in each case 10 ml of ethyl acetate and the combined extracts were dried over sodium sulfate and concentrated under reduced pressure. The crude product was purified by chromatography on reverse-phase material (Chromolith 100×5) using acetonitrile/water. Yield 60 mg. ¹H-NMR (CDCl₃) δ=2.28 (m); 3.76 (m); 4.04 (m); 5.90 (br); 6.75 (m).

### Example D

### N-Methoxy-4-chloro-6-(N-cyclopropylmethyl-N-methoxyamino)-5-(2,4,6-trifluorophenyl)-2-pyrimidinecarboximidamide (Table C, No. 27)

### Da) 4-Chloro-6-(methoxyamino)-2-methylthio-5-(2,4,6-trifluorphenyl)-pyrimidine

At room temperature, 1.41 g (16.9 mmol) of methoxyamine hydrochloride and 3.74 g (36.9 mmol) of triethylamine were added with stirring to 5.00 g (15.4 mmol) of 4,6-dichloro-2-methylthio-5-(2,4,6-trifluorophenyl)pyrimidine in 20 ml of dimethyl sulfoxide, and the mixture was then stirred at 95° C. for 7 h. The mixture was added to 250 ml of water and extracted three times with in each case 100 ml of methyl tert-butyl ether, and the combined organic phases were dried over sodium sulfate and concentrated under reduced pressure. The crude product was purified by chromatography on silica gel using cyclohexane/methyl tert-butyl ether. Yield 4.2 g.

### Db) 4-Chloro-6-(N-cyclopropylmethyl-N-methoxyamino)-2-methylthio-5-(2,4,6-trifluorphenyl)-pyrimidine

1.13 g (8.34 mmol) of cyclopropylmethyl bromide were added to 1.40 g (4.17 mmol) of 4-chloro-6-(methoxyamino)-2-methylthio-5-(2,4,6-trifluorophenyl)pyrimidine in 7 ml of dimethylacetamide, and at 0-5° C. 116 mg (4.59 mmol) of 95% pure sodium hydride was then added a little at a time, with stirring. The mixture was stirred at 0° C. for 2 h and at room temperature for 3 d, poured into 70 ml of water and extracted three times with in each case 20 ml of methyl tert-butyl ether, and the combined organic phases were dried over sodium sulfate and concentrated under reduced pressure. Yield 1.68 g.

### Dc) 4-Chloro-6-(N-cyclopropylmethyl-N-methoxyamino)-2-methylsulfonyl-5-(2,4,6-trifluorophenyl)pyrimidine

1.68 g (4.31 mmol) of 4-chloro-6-(N-cyclopropylmethyl-N-methoxyamino)-2-methylthio-5-(2,4,6-trifluorophenyl)pyrimidine were dissolved in 20 ml of methylene chloride, and at 0-5° C. 2.13 g of 3-chloroperbenzoic acid were added a little at a time with stirring. The mixture was stirred at 5° C. for 7 h and then at room temperature for 16 h and concentrated under reduced pressure, and the residue was suspended in 30 ml of ethyl acetate, washed three times with in each case 15 ml of saturated sodium bicarbonate solution, dried over sodium sulfate and concentrated under reduced pressure. The crude product was purified by chromatography on silica gel using cyclohexane/methyl tert-butyl ether. Yield 1.3 g.

### Dd) 4-Chloro-2-cyano-6-(N-cyclopropylmethyl-N-methoxyamino)-5-(2,4,6-trifluorophenyl)pyrimidine (Table B, No. 25)

At room temperature, 1.30 g (3.08 mmol) of 4-chloro-6-(N-cyclopropylmethyl-N-methoxyamino)-2-methylsulfonyl-5-(2,4,6-trifluorophenyl)pyrimidine were added to 10 ml of acetonitrile, 351 mg (5.39 mmol) of potassium cyanide and 13 mg of crown ether (18-crown-6) were added and the mixture was stirred for 16 h. The mixture was concentrated under reduced pressure, 25 ml of ethyl acetate were added, and the mixture was washed three times with in each case 10 ml of water, dried over sodium sulfate and concentrated under reduced pressure. The crude product (1.0 g) was purified by chromatography on silica gel using cyclohexane/methyl tert-butyl ether. Yield 0.90 g. ¹H-NMR (CDCl₃) δ=0.34 (m); 0.56 (m); 1.20 (m); 3.18 (s); 3.75 (d); 6.78 (m).

### De) N-Methoxy-4-chloro-6-(N-cyclopropylmethyl-N-methoxyamino)-5-(2,4,6-trifluorophenyl)-2-pyrimidinecarboximidamide

At −10° C., 5 mg (0.22 mmol) of lithium hydroxide were added with stirring to 800 mg (2.17 mmol) of 4-chloro-2-cyano-6-(N-cyclopropylmethyl-N-methoxyamino)-5-(2,4,6-trifluorophenyl)pyrimidine in 5 ml of methanol, and the mixture was stirred at −10° C. for 15 h and at room temperature for 2 d. 217 mg (2.60 mmol) of methoxyamine hydrochloride were then added, the mixture was stirred at room temperature for 18 h and concentrated under reduced pressure, 20 ml of methyl tert-butyl ether and 12 ml of saturated sodium bicarbonate solution were added, the aqueous phase was separated off and extracted with 20 ml of methyl tert-butyl ether and the combined organic phases were dried over sodium sulfate and concentrated under reduced pressure. The crude product was purified by chromatography on silica gel using cyclohexane/methyl tert-butyl ether. Yield 380 mg. ¹H-NMR (CDCl₃) δ=0.30 (m); 0.53 (m); 1.17 (m); 3.15 (s); 3.75 (d); 4.05 (s); 5.38 (br); 6.75 (m).

All experiments were carried out under protective gas (nitrogen).

TABLE C
Active compounds

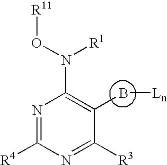

| No. | R¹¹ | R¹ | 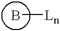 | R³ | R⁴ | Physical data |
|---|---|---|---|---|---|---|
| 1 | CH₂-cyclo(C₃H₅) | H | 2,4,6-trifluorophenyl | Cl | N | m.p. 98° C. |
| 2 | CH₂-cyclo(C₃H₅) | H | 2,4,6-trifluorophenyl | Cl | C(NH₂)═NOH | m.p. 148-151° C. |
| 3 | CH₃ | CH₃ | 2,4,6-trifluorophenyl | Cl | C(NH₂)═NOH | m.p. 190° C. |
| 4 | CH₂-cyclo(C₃H₅) | H | 2,4,6-trifluorophenyl | Cl | C(NH₂)═N—OCH₃ | ¹H-NMR (CDCl₃) δ = 0.45 |
| | | | | | | (m); 0.85(m); 3.17(d); |
| | | | | | | 4.03(s); 5.40(br.); 6.78 |
| | | | | | | (m). |
| 5 | CH₃ | CH₃ | 2,4,6-trifluorophenyl | Cl | C(NH₂)═NOCH₃ | ¹H-NMR (CDCl₃) δ = 3.10 |
| | | | | | | (s); 3.40(s); 4.02(s); 5.40 |
| | | | | | | (br.); 6.77(t). |
| 6 | CH₃ | CH₃ | 2-chloro-4-fluorophenyl | Cl | CN | m.p. 83-84° C. |
| 7 | CH₃ | CH₃ | 2-chloro-4-fluorophenyl | Cl | 1,2,4-triazol-1-yl | m.p. 144-146° C. |
| 8 | CH₃ | CH₃ | 2-chloro-4-fluorophenyl | Cl | C(NH₂)═N—OCH₃ | ¹H-NMR (CDCl₃) δ = 3.00 |
| | | | | | | (s); 3.35(s); 4.02(s); 5.40 |
| | | | | | | (br.); 7.05(m); 7.23(m). |
| 9 | CH(CH₃)₂ | CH₃ | 2-chloro-4-fluorophenyl | Cl | N | m.p. 85-90° C. |
| 10 | CH(CH₃)₂ | CH₃ | 2-chloro-4-fluorophenyl | Cl | C(NH₂)═N—OCH₃ | m.p. 164-165° C. |
| 11 | CH₃ | CH₃ | 2-chloro-4-fluorophenyl | Cl | C(═O)NH₂ | m.p. 178-180° C. |
| 12 | CH(CH₃)₂ | CH₃ | 2-chloro-4-fluorophenyl | Cl | 1,2,4-triazol-1-yl | m.p. 109-110° C. |
| 13 | CH₃ | CH₃ | 2-chloro-4-methoxyphenyl | Cl | C(NH₂)═N—OCH₃ | ¹H-NMR (CDCl₃) δ = 2.99 |
| | | | | | | (s); 3.35(s); 3.87(s); 4.05 |
| | | | | | | (s); 5.40(br.); 6.87(m); |
| | | | | | | 7.04(m); 7.15(m). |
| 14 | CH(CH₃)₂ | CH₃ | 2-chloro-4-fluorophenyl | Cl | C(═O)NH₂ | m.p. 172-174° C. |
| 15 | CH₂-cyclo(C₃H₅) | CH₃ | 2-chloro-4-fluorophenyl | Cl | 1,2,4-triazol-1-yl | m.p. 137-139° C. |
| 16 | CH₂-cyclo(C₃H₅) | CH₃ | 2-chloro-4-fluorophenyl | Cl | CN | m.p. 103-104° C. |
| 17 | —CH₂—CH₂—CH₂— | | 2,4,6-trifluorophenyl | Cl | C(NH₂)═N—OCH₃ | m.p. 192-193° C. |
| 18 | —CH₂—CH₂—CH₂— | | 2,4,6-trifluorophenyl | Cl | 1,2,4-triazol-1-yl | ¹H-NMR (CDCl₃) δ = 2.33 |
| | | | | | | (m); 3.83(m); 4.07(m); |
| | | | | | | 6.75(m); 8.15(s); 9.18 |
| | | | | | | (s). |
| 19 | —CH₂—CH₂—CH₂— | | 2,4,6-trifluorophenyl | Cl | C(═O)NH₂ | ¹H-NMR (CDCl₃) δ = 2.28 |
| | | | | | | (m); 3.76(m); 4.04(m); |
| | | | | | | 5.90(br.); 6.75(m). |
| 20 | —CH₂—CH₂—CH₂—CH₂— | | 2,4,6-trifluorophenyl | Cl | 1,2,4-triazol-1-yl | m.p. 133-134° C. |
| 21 | —CH(CH₃)—CH₂—CH₂—CH₂— | | 2,4,6-trifluorophenyl | Cl | 1,2,4-triazol-1-yl | m.p. 141-143° C. |
| 22 | —CH₂—CH₂—CH₂—CH₂— | | 2,4,6-trifluorophenyl | Cl | C(NH₂)═N—OCH₃ | m.p. 176-177° C. |
| 23 | —CH(CH₃)—CH₂—CH₂—CH₂— | | 2,4,6-trifluorophenyl | Cl | C(NH₂)═N—OCH₃ | m.p. 126-128° C. |
| 24 | CH₃ | CH(CH₃)₂ | 2,4,6-trifluorophenyl | Cl | CN | m.p. 90-91° C. |
| 25 | CH₃ | CH₂-cyclo(C₃H₅) | 2,4,6-trifluorophenyl | Cl | CN | ¹H-NMR (CDCl₃) δ = 0.34 |
| | | | | | | (m); 0.56(m); 1.20(m); |
| | | | | | | 3.18(s); 3.75(d); 6.78 |
| | | | | | | (m). |
| 26 | CH₃ | CH(CH₃)₂ | 2,4,6-trifluorophenyl | Cl | C(NH₂)═N—OCH₃ | m.p. 159-161° C. |
| 27 | CH₃ | CH₂-cyclo(C₃H₅) | 2,4,6-trifluorophenyl | Cl | C(NH₂)═N—OCH₃ | ¹H-NMR (CDCl₃) δ = 0.30 |
| | | | | | | (m); 0.53(m); 1.17(m); |
| | | | | | | 3.15(s); 3.75(d); 4.05(s); |
| | | | | | | 5.38(br.); 6.75(m). |
| 28 | CH₃ | CH₂—CH₂—OCH₃ | 2,4,6-trifluorophenyl | Cl | CN | ¹H-NMR (CDCl₃) δ = 3.17 |
| | | | | | | (s); 3.33(s); 3.62(t); 4.05 |
| | | | | | | (t); 6.76(m). |
| 29 | CH₃ | CH₂—CH₂—OCH₃ | 2,4,6-trifluorophenyl | Cl | C(NH₂)═N—OCH₃ | ¹H-NMR (CDCl₃) δ = 3.15 |
| | | | | | | (s); 3.35(s); 3.62(t); 4.05 |
| | | | | | | (s); 4.07(t); 5.35(br.); |
| | | | | | | 6.74(m). |
| 30 | —CH₂—CH₂—CH₂—CH₂— | | 2,4,6-trifluorophenyl | Cl | C(═O)NH₂ | m.p. 195-198° C. |
| 31 | CH₃ | CH₂-cyclo(C₃H₅) | 2-chloro-6-fluorophenyl | Cl | CN | 84-85° C. |
| 32 | CH₃ | CH(CH₃)₂ | 2-chloro-6-fluorophenyl | Cl | CN | ¹H NMR (400 MHz) in |
| | | | | | | CDCl₃ [ppm]; 1.3(m, 6 H); |
| | | | | | | 3.1(s, 3 H); 5.0(s, 1 H); |
| | | | | | | 7.1(t, 1 H); 7.2-7.4(m, |
| | | | | | | 2 H) |
| 33 | CH₃ | CH₂-cyclo(C₃H₅) | 2-chloro-6-fluorophenyl | Cl | C(NH₂)═N—OCH₃ | 116-118° C. |
| 34 | CH₃ | CH(CH₃)₂ | 2-chloro-6-fluorophenyl | Cl | C(NH₂)═N—OCH₃ | 152-154° C. |
| 35 | CH₃ | CH₂-cyclo(C₃H₅) | 2,6-difluorophenyl | Cl | CN | 116-118° C. |
| 36 | —CH(CH₃)—CH₂—CH₂—CH₂— | | 2-chloro-6-fluorophenyl | Cl | C(NH₂)═N—OCH₃ | 129-131° C. |
| 37 | CH₃ | CH(CH₃)₂ | 2,6-difluorophenyl | Cl | CN | 76-78° C. |
| 38 | CH₃ | CH₂-cyclo(C₃H₅) | 2,6-difluorophenyl | Cl | C(NH₂)═N—OCH₃ | 132-134° C. |
| 39 | CH₃ | CH(CH₃)₂ | 2,6-difluorophenyl | Cl | C(NH₂)═N—OCH₃ | 158-160° C. |
| 40 | CH₃ | CH₂-cyclo(C₃H₅) | 2,4,6-trifluorophenyl | Cl | OCH₃ | ¹H NMR (400 MHz) in |
| | | | | | | CDCl₃ [ppm]: 0.3(m, 2 H); |
| | | | | | | 0.5(m, 2 H); 1.2(m ,1 H); |
| | | | | | | 3.1(s, 3 H); 3.7(d, 2 H); 4.0 |
| | | | | | | (s, 3 H); 6.7(m, 2 H) |
| 41 | CH₃ | CH₂—CH═CH₂ | 2,4,6-trifluorophenyl | Cl | C(NH₂)═N—OCH₃ | ¹H NMR (400 MHz) in |
| | | | | | | CDCl₃ [ppm]: 3.1(s, 3 H); |
| | | | | | | 4.0(s, 3 H); 4.5(m, 2 H); |
| | | | | | | 5.2(m, 4 H); 5.9(m, 1 H); |
| | | | | | | 6.8(m, 2 H) |
| 42 | CH₃ | CH₂—CH₃ | 2,4,6-trifluorophenyl | Cl | C(NH₂)═N—OCH₃ | ¹H NMR (400 MHz) in |
| | | | | | | CDCl₃ [ppm]: 1.7(m, 3 H); |
| | | | | | | 3.1(s, 3 H); 3.9(m, 2 H); |
| | | | | | | 4.1(s, 3 H); 5.5(s, 2 H); |
| | | | | | | 6.7(m, 2 H) |
| 43 | CH₃ | CH₂—C₆C₅ | 2,4,6-trifluorophenyl | Cl | C(NH₂)═N—OCH₃ | ¹H NMR (400 MHz) in |
| | | | | | | CDCl₃ [ppm]: 3.2(s, 3 H); |
| | | | | | | 4.0(s, 3 H); 5.1(s, 2 H); |
| | | | | | | 5.5(s, 2 H); 6.7(m, 2 H); |
| | | | | | | 7.3(m, 5 H) |
| 44 | CH₃ | CH₂CH₂CH₃ | 2,4,6-trifluorophenyl | Cl | C(NH₂)═N—OCH₃ | ¹H NMR (400 MHz) in |
| | | | | | | CDCl₃ [ppm]: 0.9(t, 3 H); |
| | | | | | | 1.7(m, 2 H); 3.1(s, 3 H); |
| | | | | | | 3.8(t, 2 H); 4.0(s, 3 H); 5.4 |
| | | | | | | (s, 2 H); 6.7(m, 2 H) |
| 45 | CH₃ | CH(CH₃)C₂H₅ | 2-chloro-6-fluorophenyl | Cl | C(NH₂)═N—OCH₃ | ¹H NMR (400 MHz) in |
| | | | | | | CDCl₃ [ppm]: 1.9(m, 3 H); |
| | | | | | | 1.2(m, 3 H); 1.5(m ,1 H); |
| | | | | | | 1.8(m ,1 H); 3.2(s, 3 H); |
| | | | | | | 4.0(s, 3 H); 4.6(m, 1 H); |
| | | | | | | 5.3(s, 2 H); 7.1(m, 1 H); |
| | | | | | | 7.3(m, 2 H) |
| 46 | CH₃ | CH₂CH(CH₃)₂ | 2-chloro-6-fluorophenyl | Cl | C(NH₂)═N—OCH₃ | |
| 47 | CH₃ | cyclo-(C₅H₉) | 2-chloro-6-fluorophenyl | Cl | C(NH₂)═N—OCH₃ | |
| 48 | CH₃ | CH₂OCH₃ | 2-chloro-6-fluorophenyl | Cl | C(NH₂)═N—OCH₃ | |

| | | | | | |
|---|---|---|---|---|---|
| 49 | —CH₂—CH₂—CH(CH₃)—CH₂— | 2,4,6-trifluorophenyl | Cl | C(NH₂)═N—OCH₃ | 119-120° C. |

### Examples of the Action Against Harmful Fungi

### A Greenhouse Experiments

The active compounds were prepared separately as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### Use Example 1

### Activity Against Early Blight on Tomatoes Caused by Alternaria solani

Leaves of potted plants of the cultivar “Goldene Königin” were sprayed to runoff point with an aqueous suspension having the concentration of active compound stated below. The next day, the leaves were infected with an aqueous spore suspension of Alternaria solani in a 2% biomalt solution having a density of 0.17×10⁶ spores/ml. The plants were then placed in a water vapor-saturated chamber at temperatures between 20 and 22° C. After 5 days, the disease on the untreated, but infected control plants had developed to such an extent that the infection could be determined visually in %.

The plants which had been treated with an application rate of in each case 250 ppm of the compounds 3 to 5, 7, 8, 10 to 13, 15, 17, 18, 20 to 27, 30, 36, 41, 42, 43 or 46 to 49 of Table C showed an infection of at most 20%, whereas the untreated control plants were 90% infected.

The plants treated with the compounds 33 and 34, respectively, of Table C at an application rate of in each case 63 ppm showed an infection of at most 7%, whereas the untreated control plants were 90% infected.

### Use Example 2

### Curative Activity Against Brown Rust of Wheat Caused by Puccinia recondita

Leaves of potted wheat seedlings of the cultivar “Kanzler” were inoculated with a spore suspension of brown rust (Puccinia recondita). The pots were then placed into a chamber with high atmospheric humidity (90-95%) at 20-22° C. for 24 hours. During this time, the spores germinated and the germ tubes penetrated into the leaf tissue. The next day, the infected plants were sprayed to runoff point with the active compound solution described above having the concentration of active compound stated below. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

The plants which had been treated with an application rate of 250 ppm of the compound 9 showed an infection of 10%, whereas the untreated control plants were 80% infected.

The plants which had been treated with an application rate of in each case 250 ppm of the compounds 37 to 39, 44 and 45, respectively, showed an infection of at most 10%, whereas the untreated control plants were 90% infected.

The plants which had been treated with an application rate of 63 ppm of the compound 33 showed an infection of 0%, whereas the untreated control plants were 90% infected.

### Use Example 3

### Activity Against Late Blight on Tomatoes Caused by Phytophthora infestans, Protective Treatment

Leaves of potted tomato plants were sprayed to runoff point with an aqueous suspension having the concentration of active compound stated below. The next day, the leaves were infected with an aqueous sporangia suspension of Phytophthora infestans. The plants were then placed in a water vapor-saturated chamber at temperatures between 18 and 20° C. After 6 days, the late blight on the untreated, but infected control plants had developed to such an extent that the infection could be determined visually in %.

The plants which had been treated with an application rate of in each case 250 ppm of the compounds 24, 25, 28, 29 or 31 showed an infection of at most 20%, whereas the untreated control plants were 90% infected.

### Use Example 4

### Activity Against Net Blotch of Barley Caused by Pyrenophora teres, 1 Day Protective Application

Leaves of potted barley seedlings were sprayed to runoff point with an aqueous suspension having the concentration of active compound stated below. 24 hours after the spray coating had dried on, the test plants were inoculated with an aqueous spore suspension of Pyrenophora [syn. Drechslera] teres, the net blotch pathogen. The test plants were then placed into a greenhouse at temperatures between 20 and 24° C. and 95 to 100% relative atmospheric humidity. After 6 days, the extent of the development of the disease was determined visually in % infection of the total leaf area.

The plants which had been treated with an application rate of in each case 250 ppm of the compounds 3 to 5, 7, 8, 11 to 15, 18 to 22, 26 and 27 showed an infection of at most 20%, whereas the untreated control plants were 90% infected.

### Use Example 5

### Protective Activity Against Puccinia recondita on Wheat (Brown Rust of Wheat)

Leaves of potted wheat seedlings of the cultivar “Kanzler” were sprayed to runoff point with an aqueous suspension having the concentration of active compound stated below. The next day, the treated plants were inoculated with a spore suspension of brown rust of wheat (Puccinia recondita). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and at 65 to 70% relative atmospheric humidity for a further 7 days. The extent of the rust fungus development on the leaves was then determined visually.

The plants which had been treated with an application rate of in each case 250 ppm of the compounds 9, 17, 23, 19, 28, 36 to 39, 41, 42 or 44 to 49 showed an infection of at most 15%, whereas the untreated control plants were 90% infected.

### Use Example 6

### Activity Against Mildew of Wheat Caused by Erysiphe [syn. Blumeria] graminis Forma Specialis, tritici

Leaves of potted wheat seedlings were sprayed to runoff point with an aqueous suspension having the concentration of active compound stated below. The suspension or emulsion had been prepared as described above. 24 hours after the spray coating had dried on, dusted with spores of mildew of wheat (Erysiphe [syn. Blumeria] graminis forma specialis. tritici). The test plants were then placed in a greenhouse at temperatures between 20 and 24° C. and 60 to 90% relative atmospheric humidity. After 7 days, the extent of the mildew development was determined visually in % infection of the total leaf area.

The plants which had been treated with an application rate of 250 ppm of the compound 35 showed an infection of 5%, whereas the control plants were 90% infected.

### B Microtest

The active compounds were formulated separately as a stock solution having a concentration of 10 000 ppm in DMSO.

Microtest No. 1—Activity Against the Septoria Leaf Spot Pathogen Septoria tritici in the Microtiter Test

The stock solution was pipetted into a microtiter plate (MTP) and diluted with an aqueous malt-based fungus nutrient medium to the stated active compound concentration. An aqueous spore suspension of Septoria tritici was then added. The plates were placed in a water vapor-saturated chamber at temperatures of 18° C. On day 7 after the inoculation, the MTPs were measured in an absorption photometer at 405 nm. The measured parameters were compared to the growth of the active compound-free control variant (100%) and the fungus- and active compound-free blank value to determine the relative growth in % of the pathogens in the individual active compounds.

At a concentration of in each case 125 ppm, the compounds 32 and 40, respectively, caused a relative growth of 0%.

Microtest No. 2—Activity Against the Rice Blast Pathogen Pyricularia oryzae in the Microtiter Test

The stock solution was pipetted into a microtiter plate (MTP) and diluted with an aqueous malt-based fungus nutrient medium to the stated active compound concentration. An aqueous spore suspension of Pyricularia oryzae was then added. The plates were placed in a water vapor-saturated chamber at temperatures of 18° C. On day 7 after the inoculation, the MTPs were measured in an absorption photometer at 405 nm. The measured parameters were compared to the growth of the active compound-free control variant (100%) and the fungus- and active compound-free blank value to determine the relative growth in % of the pathogens in the individual active compounds.

At a concentration of in each case 125 ppm, the compounds 32 and 40, respectively, caused a relative growth of 0%.

1-21. (canceled)

22. A compound of formula I, 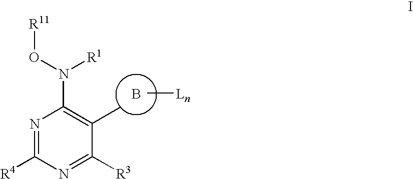
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a}—NR^{z}R^{b}, C(=Z)R^{a}, CR^{a}R^{b}—OR^{e}, CR^{a}R^{k}—NR^{z}R^{c}, ON(═CR^{a}R^{b}), O—C(=Z)R^{a}, NR^{a}R^{b′}, NR^{a}(C(=Z)R^{b}) NR^{a}(C(=Z)OR^{b}), NR^{a}(C(=Z)-NR^{z}R^{b}), NR^{a}(N═CR^{c}R^{b}), NR^{a}—NR^{b}R^{b}, NR^{z}—OR^{a}; wherein Z is O, S, NR^{d}, NOR^{d} or N—NR^{z}R^{c};
R^{b′} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d}; wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 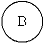
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1,2,3,4 or 5;
or an agriculturally acceptable salt of a compound of formula I.

23. The compound of claim 22, having the formula I′ 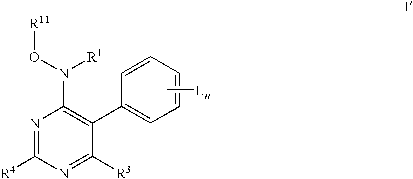
wherein:
R¹ and R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl, wherein R¹ and R¹¹ together may also form a five-, six- or seven-membered saturated or unsaturated heterocycle; wherein
R¹ and/or R¹¹ or a heterocycle formed by R¹ and R¹¹ may carry one, two, three or four identical or different substituents R¹, wherein R² is:
R² halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, wherein the phenyl moiety may carry one, two or three radicals independently of one another selected from the group consisting of: halogen, C₁-C₆-alkyl, C₂-C₆-alkenyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
A, A′, A″ independently of one another hydrogen, C₁-C₆-alkyl, C₂-C₆-alkenyl, phenyl, wherein the organic radicals may be partially or fully halogenated and/or may be substituted by C₁-C₄-alkoxy; A and A′ together with the atoms to which they are attached may also be a five- or six-membered saturated heterocycle which contains one or two heteroatoms from the group consisting of O, N and S;
R³ is halogen, cyano, C₁-C₄-alkyl, C₁-C₄-alkoxy, C₁-C₄-haloalkoxy or C₁-C₄-haloalkyl;
R⁴ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furyl, thienyl, thiazolyl, isothiazolyl, pyridinyl, pyrimidinyl, pyrazinyl, pyridazinyl, 1,2,3-triazinyl, 1,2,4-triazinyl, 1-pyridin(1,2-dihydro)-2-onyl or 1-pyrrolidonyl, wherein R⁴ is unsubstituted or substituted by one, two or three substituents R^{u}:
R^{u} is halogen, cyano, C₁-C₈-alkyl, C₁-C₆-alkoxy, —C(═O)-A, —C(═O)—O-A, —C(═O)—N(A′)A, C(A′)(═N—OA), N(A′)A, N(A′)-C(═O)-A; wherein A, A′ are as defined above; or cyano, C(═O)NR^{z}R^{b}, C(═O)OR^{a}, C(═NOR^{a})NR^{z}R^{b}, C(═NOR^{b})R^{a}, C(═N—NR^{z}R^{b})R^{a} or CR^{a}R^{b}—N^{z}R^{c}, ON(═CR^{a}R^{b}), NR^{a}(C(═O)R^{b}), NR^{a}(C(═O)OR^{b}), NR^{a}(N═CR^{c}R^{b}) or NR^{z}—OR^{a};
L is halogen, cyano, methyl, methoxy, —C(═O)—O-A, —C(═O)—N(A′)A, —C(═S)—N(A′)A, C(A′)(═N—OA), N(A′)A, N(A′)-C(═O)-A; wherein the aliphatic groups of the radical definitions of L for their part may be partially or fully halogenated; and
n is 1, 2 or 3, wherein at least one substituent L on the phenyl ring is located in the ortho-position to the point of attachment to the pyrimidine.

24. The compound of claim 23, wherein:
R¹ and R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₂-C₆-alkynyl, C₁-C₆-haloalkyl, C₂-C₆-haloalkenyl or C₂-C₆-haloalkynyl, wherein R¹ and R¹¹ together may also form a five-, six- or seven-membered saturated or unsaturated heterocycle; wherein
R¹ and/or R¹¹ or a heterocycle formed by R¹ and R¹¹ may carry one, two, three or four identical or different substituents R², wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, wherein the phenyl moiety may carry one, two or three radicals independently of one another selected from the group consisting of: halogen, C₁-C₆-alkyl, C₂-C₆-alkenyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
A, A′, A″ independently of one another hydrogen or C₁-C₆-alkyl which may be partially or fully halogenated and/or may be substituted by C₁-C₄-alkoxy;
R³ is halogen, cyano, C₁-C₄-alkyl, C₁-C₄-alkoxy, halogenmethoxy or C₁-C₄-haloalkyl;
R⁴ is pyrazolyl, 1,2,3-triazolyl or 1,2,4-triazolyl, wherein R⁴ is attached to the pyrimidine ring via N and is unsubstituted or substituted by one or two substituents R^{u}:
R^{u} is halogen, cyano, C₁-C₄-alkyl, C₁-C₄-alkoxy, —C(═O)—O-A, —C(═O)—N(A′)A, C(A′)(═N—OA), N(A′)A, N(A′)-C(═O)-A; wherein A,A′ are as defined above; R⁴ may furthermore be: C(═O)NR^{z}R^{b}, C(═O)OR^{a}, C(═NOR^{a})NH₂, C(═NOR^{b})R^{a} or NR^{a}(C(═O)OR^{b}); wherein:
R^{a}, R^{b} and R^{c} independently of one another are hydrogen, C₁-C₆-alkyl, or C₃-C₆-cycloalkyl;
R^{z} has the same meanings as R^{a} and may additionally be —CO—R^{d} or —COO—R^{d};
L is halogen, cyano, methyl, methoxy, —C(═O)—O-A, —C(═O)—N(A′)A, —C(═S)—N(A′)A, C(A′)(—N—OA), N(A′)A, N(A′)-C(═O)-A; wherein A and A′ are as defined above; and
n is 1, 2 or 3, wherein at least one substituent L on the phenyl ring is located in the ortho-position to the point of attachment to the pyrimidine.

25. The compound of claim 22, wherein:
R⁴ is 1-pyrazolyl or 1-[1,2,4]triazolyl.

26. The compound of claim 22, wherein:
R⁴ is 2-pyridinyl, 3-pyridazinyl, 1-pyridin(1,2-dihydro)-2-onyl or 1-pyrrolidonyl.

27. The compound of claim 22, wherein:
R⁴ is 2-pyrimidinyl.

28. The compound of claim 22, wherein:
R⁴ is C(=Z)OR^{a}, C(=Z)NR^{z}R^{b} or C(=Z)R^{a} and
Z is O, NR^{d} or NOR^{d}.

29. The compound of claim 22, wherein:
R¹ and R¹¹ independently of one another are C₁-C₆-alkyl, C₃-C₆-cycloalkyl, C₁-C₄-alkyl-C₃-C₆-cycloalkyl, C₃-C₆-cycloalkyl-C₁-C₆-alkyl, di-C₁-C₄-alkyl-C₃-C₆-cycloalkyl, C₁-C₄-alkoxy-C₁-C₆-alkyl, C₂-C₆-alkenyl, C₁-C₆-haloalkyl or C₂-C₆-haloalkenyl.

30. The compound of claim 22, wherein:
R¹ and R¹¹ together form a five-, six- or seven-membered saturated or unsaturated heterocycle which may carry one, two, three or four identical or different substituents R², wherein:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₃-C₆-cycloalkyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, or phenyl, wherein the phenyl moiety may carry one, two or three radicals independently of one another selected from the group consisting of: halogen, C₁-C₆-alkyl, C₂-C₆-alkenyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
A, A′, A″ independently of one another hydrogen, C₁-C₆-alkyl, C₂-C₆-alkenyl, phenyl, wherein the organic radicals may be partially or fully halogenated and/or may be substituted by C₁-C₄-alkoxy; A and A′ together with the atoms to which they are attached may also be a five- or six-membered saturated heterocycle which contains one or two heteroatoms from the group consisting of O, N and S.

31. The compound of claim 30, wherein:
R¹ and R¹¹ together form a saturated five- or six-membered ring which may carry one, two, three or four identical or different substituents R².

32. The compound of claim 31, wherein R² is C₁-C₆-alkyl or C₁-C₆-haloalkyl.

33. The compound of claim 32, wherein: B is substituted by Lₙ is of the formula: 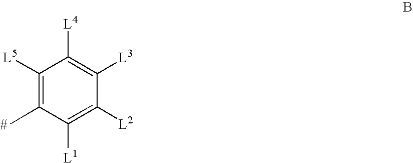
wherein # is the point of attachment to the pyrimidine, and
L¹ is fluorine, chlorine, CH₃ or CF₃;
L² and L⁴ independently of one another are hydrogen, CH₃ or fluorine;
L³ is hydrogen, fluorine, chlorine, cyano, nitro, CH₃, SCH₃, OCH₃, SO₂CH₃, NH—C(═O)CH₃, N(CH₃)—C(═O)CH₃, C(═S)NH₂ or COOCH₃, and
L⁵ is hydrogen, fluorine, chlorine or CH₃.

34. The compound of claim 22, having the formula Ia 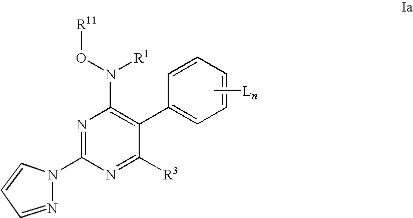
wherein, Lₙ is 2-fluoro, 6-chloro, R³ is methyl, R¹¹ is CH₃ and R¹¹ is CH₃.

35. A pesticidal composition comprising at least one compound of formula I 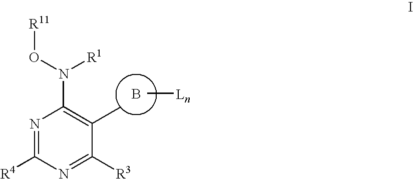
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c}, ON(═CR^{a}R^{b}), O—C(=Z)R^{a}, NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}; wherein Z is O, S, NR^{d}, NOR^{d} or N—NR^{z}R^{c};
R^{b} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d};
wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 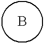
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1, 2, 3, 4 or 5;
and/or an agriculturally acceptable salt thereof,
and at least one solid or liquid carrier.

36. The pesticidal composition of 35, further comprising at least one fungicidally, insecticidally and/or herbicidally active compound.

37. A composition of at least one compound of formula I 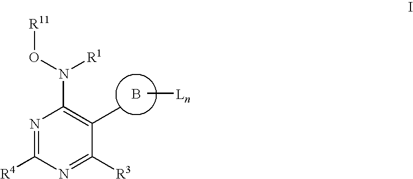
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O), —N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c}, ON(═CR^{a}R^{b}), O—C(=Z)R^{a}, NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}; wherein Z is O, S, NR^{d}, NOR^{d} or N—NR^{z}R^{c};
R^{b′} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d}; wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 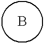
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1,2,3, 4 or 5;
and/or an agriculturally acceptable salt thereof,
and at least one further fungicidally, insecticidally and/or herbicidally active compound.

38. The composition of claim 37, comprising a compound of formula Ia 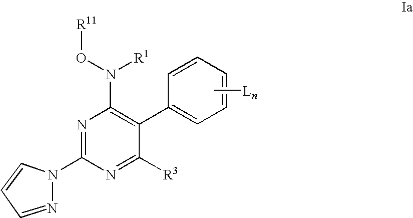
wherein Lₙ is 2-fluoro, 6-chloro, R³ is methyl, R¹ is CH₃ and R¹¹ is CH₃, and the said at least one further active is azoxystrobin.

39. The composition of claim 37, comprising a compound formula I, wherein R¹¹ is CH₃, R¹ is CH₃, 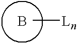
is 2-Chloro-4-fluorophenyl, R³ is Cl and R⁴ is 1,2,4-triazol-1-yl, and the at least one further active is orysastrobin.

40. A method for controlling phytopathogenic fungi comprising,
treating the fungi and/or the materials, plants, the soil or seed to be protected against fungal attack with an effective amount of at least one compound of formula I 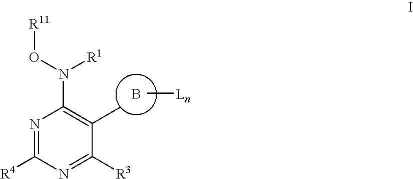
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a}—NR^{z}R^{b}, C(=Z)R^{a}, CR^{a}R^{b}—OR^{z}, CR^{a}R^{b}—NR^{z}R^{c}, ON(═CR^{a}R^{b}), O—C(=Z)R^{a}, NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}; wherein Z is O, S, NR^{d}, NOR^{d} or N—NR^{z}R^{c};
R^{b} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d}; wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 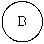
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1,2,3,4 or 5;
and/or an agriculturally acceptable salt thereof.

41. A method for controlling animal pests in agriculture comprising,
treating the pests and/or the materials, plants, the soil or seed to be protected against fungal attack with an effective amount of at least one compound of formula I 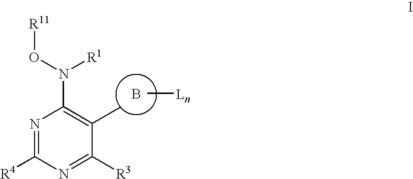
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a} NR^{z}R^{b}, C(=Z)R^{a}, CR^{a}R^{b}—OR^{z}, CR^{a}R^{b}—NR^{z}R^{c}, ON(═CR^{a}R^{b}), O—C(=Z)R^{a}, NR^{a}R^{b′}, NR^{a}(C(=Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}—OR^{a}; wherein Z is O, S, NR^{d}, NOR^{d} or N—NR^{z}R^{c};
R^{b} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d}; wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 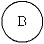
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1, 2, 3, 4 or 5;
and/or an agriculturally acceptable salt thereof.

42. A seed comprising in an amount of from 1 to 1000 g per 100 kg a compound of formula I 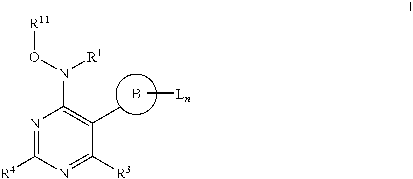
wherein:
R¹, R¹¹ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl; C₄-C₆-cycloalkenyl or C₄-C₆-halocycloalkenyl;
R¹ and R¹¹ together with the atoms to which they are attached may also form a five-, six- or seven-membered saturated or unsaturated heterocycle which may contain a further heteroatom from the group consisting of O, N and S as ring member, wherein
R¹ and/or R¹¹ or a heterocycle formed by R¹ and R¹¹ may carry one, two, three or four identical or different substituents R² and/or two substituents attached to adjacent ring atoms may be C₁-C₆-alkylene, oxy-C₂-C₄-alkylene or oxy-C₁-C₃-alkyleneoxy; wherein R² is:
R² is halogen, cyano, C₁-C₆-alkyl, C₁-C₆-haloalkyl, C₃-C₆-cycloalkyl, C₃-C₆-halocycloalkyl, C₄-C₆-cycloalkenyl, hydroxyl, C₁-C₆-alkoxy, C₁-C₆-haloalkoxy, C₂-C₈-alkenyloxy, C₂-C₈-alkynyloxy, C₃-C₆-cycloalkyloxy, C₄-C₆-cycloalkenyloxy, C₁-C₆-alkylthio, ═CH₂, ═CH(C₁-C₄-alkyl), ═C(C₁-C₄-alkyl)₂, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃ or phenyl, wherein the phenyl moiety may carry one, two or three radicals independently of one another selected from the group consisting of: halogen, C₁-C₆-alkyl, C₂-C₆-alkenyl, C₂-C₆-alkynyl, C₃-C₆-cycloalkyl, C₁-C₆-haloalkyl, C₁-C₆-alkoxy, cyano, nitro, —C(═O)-A, —C(═O)—O-A, —C(═O)—N(A′)A, C(A′)(═N—OA), N(A′)A; wherein,
m 0, 1 or 2;
A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, phenyl, wherein the organic radicals may be partially or fully halogenated and/or may be mono- or polysubstituted by nitro, cyanato (OCN), cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
R³ is halogen, cyano, azido, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₃-C₄-alkenyloxy, C₃-C₄-alkynyloxy, C₁-C₆-alkylthio, di-(C₁-C₆-alkyl)amino or C₁-C₆-alkylamino, wherein the aliphatic and alicyclic groups of the radical definitions of R³ for their part may contain one, two, three or four substituents independently of one another selected from the group consisting of halogen, cyano, nitro, C₁-C₂-alkoxy and C₁-C₄-alkoxycarbonyl;
R⁴ is a five- or six-membered saturated, partially unsaturated or aromatic heterocycle which contains one, two, three or four heteroatoms from the group consisting of O, N and S, wherein R⁴ may be partially or fully halogenated and/or may carry one, two, three or four identical or different groups R^{u}:
R^{u} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
R⁴ may furthermore be: cyano, C₁-C₈-alkoxy, C(=Z)OR^{a}, C(=Z)NR^{z}R^{b}, C(=Z)NR^{a}—NR^{z}R^{b}, C(=Z)R^{a}, CR^{a}R^{b}OR^{z}, CR^{a}R^{b}—NR^{z}R^{c}, ON(═CR^{a}R^{b}), O—C(=Z)R^{a}, NR^{a}R^{b′}, NR^{a}(C(Z)R^{b}), NR^{a}(C(=Z)OR^{b}), NR^{a}(C(=Z)-NR^{z}R^{b}), NR^{a}(N═CR^{c}R^{b}), NR^{a}—NR^{z}R^{b}, NR^{z}-OR^{a}; wherein Z is O, S, NR^{d}, NOR^{d} or N—NR^{z}R^{c};
R^{b′} is C₁-C₆-alkyl, C₂-C₆-alkenyl, C₂-C₆-alkynyl, C₃-C₆-cycloalkyl or C₄-C₆-cycloalkenyl;
R^{a}, R^{b}, R^{c} and R^{d} independently of one another are hydrogen or have one of the meanings mentioned for R^{b′};
R^{z} has the same meanings as R^{a} and may additionally be —CO—R^{d} or —COO—R^{d}; wherein the aliphatic and alicyclic groups of the radical definitions of R^{a}, R^{b}, R^{c}, R^{d}, R^{b′} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy;
and wherein two of the radicals R^{a}, R^{b}, R^{c}, R^{z} together with the atoms to which they are attached may form a five- or six-membered saturated, partially unsaturated or aromatic heterocycle which may contain one, two, three or four heteroatoms from the group consisting of O, N and S; 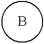
is five- or six-membered heteroaryl which contains one, two, three or four heteroatoms selected from the group consisting of O, N and S, or is phenyl;
L is halogen, cyano, cyanato (OCN), C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₂-C₈-alkenyloxy, C₂-C₈-alkynyloxy, C₃-C₆-cycloalkyl, C₃-C₆-halocycloalkyl, C₄-C₆-cycloalkenyl, C₃-C₆-cycloalkyloxy, C₄-C₆-cycloalkenyloxy, nitro, —C(═O)-A, —C(═O)—O-A, —C(═O)—N(A′)A, —C(═S)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above; wherein the aliphatic and alicyclic groups of the radical definitions of L may carry one, two, three or four groups R^{L}:
R^{L} is halogen, cyano, C₁-C₆-alkoxy, C₃-C₆-cycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A or S(═O)ₘ—N(A′)A; wherein m, A, A′, A″ are as defined above;
and
n is 1, 2, 3, 4 or 5;
and/or an agriculturally acceptable salt thereof.
